(19) United States
(12) Patent Application Publication
(10) Pub. No.: US 2014/0147848 A1
(43) Pub. Date: May 29, 2014

(54) COMPOSITIONS AND METHODS FOR LINEAR ALKYLBENZENE SULFONATE (LAS) RISK ASSESSMENT

(71) Applicants: UNIVERSITY OF TSUKUBA, Ibaraki (JP); CENTER OF BIOTECHNOLOGY OF SFAX, Safax, TN (US)

(72) Inventors: Hiroko Isoda, Tsukuba (JP); Junkyu Han, Tsukuba (JP); Sayadi Sami, Sfax (TN); Mohamed Bradai, Tsukuba (JP)

(73) Assignees: CENTER OF BIOTECHNOLOGY OF SFAX, Safax, TN (US); UNIVERSITY OF TSUKUBA, Ibaraki (JP)

(21) Appl. No.: 13/685,354

(22) Filed: Nov. 26, 2012

(51) Int. Cl. G01N 33/68

(52) U.S. Cl. CPC G01N 33/68; G06F 19/10
USPC 435/6.12; 435/24; 435/29; 702/19

(57) ABSTRACT

The present disclosure provides a method for assessing the environmental effects of alkylbenzenesulfonate (LAS). For example, the method includes contacting a population of cells with a sample, measuring an expression level of one or more LAS biomarkers in the cell population, comparing the level of expression of the one or more LAS biomarker to one or more reference values corresponding to the one or more LAS biomarkers, and determining an LAS risk associated with the sample.

### BACKGROUND OF THE INVENTION

Linear alkylbenzenesulfonate (LAS)—also known as sodium dodecylbenzenesulfonate or dodecylbenzenesulfonic acid, sodium salt—belongs to a family of compounds known as alkylbenzenesulfonates, which have the general formula C₁₂H₂₅C₆H₄SO₃Na. Alkylbenzenesulfonates may be produced by a variety of methods, but are typically produced by alkylating benzene with long chain monoalkenes (such as, e.g., dodecene) and using hydrogen fluoride as a catalyst. The resulting dodecylbenzene molecules are purified and then sulfonated with sulfur trioxide to produce the sulfonic acid, which is subsequently neutralized with sodium hydroxide. In LAS molecules, the C₁₂H₂₅ dodecyl group is unbranched.

LAS is one of the major anionic surfactants used in detergents such as, for example, laundry powders, laundry liquids, dishwashing products, all-purpose cleaners, etc. Current estimates indicate that the total annual consumption of LAS is approximately 430 kilotons, of which nearly 350 kilotons is derived from household use. After use, such detergent compounds are typically discharged into the environment (e.g., in wastewater). This is problematic because LAS is known to be hazardous/toxic to humans, and also to a variety of flora and fauna naturally occurring in the environment (e.g., bacteria, aquatic animals, etc.). Given that LAS is primarily used in detergents, these hazardous/toxic characteristics are of particular concern because such detergents are frequently used either directly in environmental applications (e.g., oil cleanup) or in residential/commercial applications that result in the detergents being disposed into the sewage system, where they may have direct access to the water stream, depending on local sewage treatment practices and on the characteristics of the receiving environment. Current methods of assessing the risk of LAS are based on the detection of the chemical concentration of the compound (e.g., purified LAS, LAS within a complex solution such as wastewater, etc.) and not its effect; therefore, these conventional methods do not have the ability to assess the human/animal/environmental impact of LAS contamination. Moreover, little is known about what types of synergistic toxicological effects LAS may exert when combined with other environmental contaminants. Accordingly, there is a need to develop new methods for assessing the actual risk and effect of LAS compounds, including effects resulting from the interaction and/or combination of LAS with other factors (e.g., other chemicals, contaminants, naturally occurring chemicals, flora, fauna, etc.), and including this in the risk assessment methods.

### SUMMARY OF THE INVENTION

As described below, the present invention features compositions and methods for cytotoxic effect measurement and risk assessment of linear alkylbenzenesulfonate (LAS) in the environment, and more particularly, an aqueous environment.

In one aspect, the present invention provides a method, including contacting a population of cells with a sample, measuring an expression level of one or more linear alkylbenzenesulfonate (LAS) biomarkers in the cell population, comparing the level of expression of the one or more LAS biomarker to one or more reference values corresponding to the one or more LAS biomarkers; and determining an LAS risk associated with the sample.

In one exemplary embodiment, the population of cells is a population of Caco-2 cells. In another exemplary embodiment, the one or more LAS biomarkers are selected from the group consisting of tropomyosin alpha-3 chain (TPM3), thioredoxin (THIO), heat shock cognate 71 kDa (HSP7C), and calreticulin (CALR).

In another embodiment, the expression level of the one or more LAS biomarkers corresponds to a mRNA level or a protein level.

In yet another embodiment, determining an LAS risk further comprises calculating the LAS risk according to Formula (I)

$$Risk = \frac{P E C}{P N E C} + ( \frac{Exp 1 - Ref 1}{Ref 1} + \frac{Exp 2 - Ref 2}{Ref 2} + \frac{Exp 3 - Ref 3}{Ref 3} + \frac{Exp 4 - Ref 4}{Ref 4} ) / 4 , \qquad Formula ( I )$$

where, PEC is a Predicted Environmental Concentration, PNEC is a Predicted No Effect Concentration; Exp1 is a TPM3 expression level in the cell population; Ref1 is a TPM3 expression level in a standard; Exp2 is an HSP7C expression level in the cell population; Ref2 is a HSP7C expression level in a standard; Exp3 is a CALR expression level in the cell population; Ref3 is a CALR expression level in a standard; Exp4 is a THIO expression level in the cell population; and Ref4 is a THIO expression level in a standard.

In another embodiment, the sample is selected from the group consisting of a water sample, a soil sample, and a sewage sample.

In another aspect, the present invention discloses a method including contacting a cell with a sample, measuring a level of RNA expression of one or more linear alkylbenzenesulfonate (LAS) biomarkers; and comparing the level of RNA expression of the one or more LAS biomarkers to a reference value for each of the one or more LAS biomarkers to determine presence or absence of an LAS risk in the sample.

In one embodiment, the population of cells is a population of Caco-2 cells.

In another embodiment, the one or more LAS biomarkers are selected from the group consisting of tropomyosin alpha-3 chain (TPM3), thioredoxin (THIO), heat shock cognate 71 kDa (HSP7C), and calreticulin (CALR).

In another embodiment, determining an LAS risk further comprises calculating the LAS risk according to Formula (I)

$$Risk = \frac{P E C}{P N E C} + ( \frac{Exp 1 - Ref 1}{Ref 1} + \frac{Exp 2 - Ref 2}{Ref 2} + \frac{Exp 3 - Ref 3}{Ref 3} + \frac{Exp 4 - Ref 4}{Ref 4} ) / 4 , \qquad Formula ( I )$$

Where, PEC is a Predicted Environmental Concentration, PNEC is a Predicted No Effect Concentration; Exp1 is a TPM3 expression level in the cell population; Ref1 is a TPM3 expression level in a standard; Exp2 is an HSP7C expression level in the cell population; Ref2 is a HSP7C expression level in a standard; Exp3 is a CALR expression level in the cell population; Ref3 is a CALR expression level in a standard; Exp4 is a THIO expression level in the cell population; and Ref4 is a THIO expression level in a standard.

In another embodiment, the sample is selected from the group consisting of a water sample, a soil sample, and a sewage sample.

### DEFINITIONS

Unless defined otherwise, all technical and scientific terms used herein have the meaning commonly understood by a person skilled in the art to which this invention belongs. The following references provide one of skill with a general definition of many of the terms used in the disclosure: Singleton et al., Dictionary of Microbiology and Molecular Biology (2^{nd} ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Calreticulin (CALR)” is meant a polypeptide or fragment thereof having at least about 85% amino acid identity to NCBI Accession No. P27797, as shown in Table 1, and having calcium-binding chaperone activity that promotes folding, oligomeric assembly, and quality control of proteins in the endoplasmic reticulum (ER), as well as a regulatory activity for the regulation of calcium homeostasis.

TABLE 1
CALR Polypeptide (P27797) (SEQ ID NO: 1)

| | |
|---|---|
| 1 | MLLSVPLLLG LLGLAVAEPA VYFKEQFLDG DGWTSRWIES |
| | KHKSDFGKFV |
| 51 | LSSGKFYGDE EKDKGLQTSQ DARFYALSAS FEPFSNKGQT |
| | LVVQFTVKHE |
| 101 | QNIDCGGGYV KLFPNSLDQT DMHGDSEYNI MFGPDICGPG |
| | TKKVHVIFNY |
| 151 | KGKNVLINKD IRCKDDEFTH LYTLIVRPDN TYEVKIDNSQ |
| | VESGSLEDDW |
| 201 | DFLPPKKIKD PDASKPEDWD ERAKIDDPTD SKPEDWDKPE |
| | HIPDPDAKKP |
| 251 | EDWDEEMDGE WEPPVIQNPE YKGEWKPRQI DNPDYKGTWI |
| | HPEIDNPEYS |
| 301 | PDPSIYAYDN FGVLGLDLWQ VKSGTIFDNF LITNDEAYAE |
| | EFGNETWGVT |
| 351 | KAAEKQMKDK QDEEQRLKEE EEDKKRKEEE EAEDKEDDED |
| | KDEDEEDEED |
| 401 | KEEDEEEDVP GQAKDEL |

By “Calreticulin nucleic acid molecule” is meant a polynucleotide encoding a CALR polypeptide. An exemplary CALR nucleic acid molecule is provided at NCBI Accession No. NC_{—}000019.9, and is also shown below.

CALR Nucleic Acid Sequence (NC_000019.9)
(SEQ ID NO: 2)
GCGGCGTCCGTCCGTACTGCAGAGCCGCTGCCGGAGGGTCGTTTTAAAGGGCCCGCGCGTTGCCGCCCCC
TCGGCCCGCCATGCTGCTATCCGTGCCGCTGCTGCTCGGCCTCCTCGGCCTGGCCGTCGCCGAGCCTGCC
GTCTACTTCAAGGAGCAGTTTCTGGACGGAGGTAACGCCTGGTCCCGCCTCGAGGCCGCCCCGACGACGC
GGCCGGCCCCCGATCCTGGATCTGCGTTGTCGCCCGTAATTACCGTTTAGAGGTCCAACACGGTGGCCTC
CCGGGACTAGAGCCGCGGGCGATTTCTCTTCTGCGTCCCTGGGGAGCGCGGAGGGCGTAGCGGCCTCCCG
CGGCGGGAGTTAGGGTTAGCCCGAGGATCTCTGAAGGCACCCGACGTGTCAAACTAGAGGTTGGAATGGG
GAGTGTCGGGGATCTCCTTTCCTGTCCCCAGCAGCTTGTGGCTCTCGGCAGATGTTTGGTGTGGGGGGGG
ATTAGCACAGCCGCTCTGACCTACCCCTCTAATCCCCCACTTAGACGGGTGGACTTCCCGCTGGATCGAA
TCCAAACACAAGTCAGATTTTGGCAAATTCGTTCTCAGTTCCGGCAAGTTCTACGGTGACGAGGAGAAAG
ATAAAGGTAAGAGCCTAGGAGTGGGTGCTCAGATCCGGGAGGACTTCCTGGCAGAAGTCCTTGTCTGTAC
ACACACAGCCGGGACAGTCCCCTTGGAGGAGGACAGGTGGAGGAAGTGGGGGAGTCTTCTCTATTCTCTA
AGTCGAGGGTCCTCGCGAGTCAAGGCCCAACGGTGACCTCACTACCGTCCCGTCTCAGGTTTGCAGACAA
GCCAGGATGCACGCTTTTATGCTCTGTCGGCCAGTTTCGAGCCTTTCAGCAACAAAGGCCAGACGCTGGT
GGTGCAGTTCACGGTGAAACATGAGCAGAACATCGACTGTGGGGGCGGCTATGTGAAGCTGTTTCCTAAT
AGTTTGGACCAGACAGACATGCACGGAGACTCAGAATACAACATCATGTTTGGTGAGGGCCTGCTTCCTG
GTGCTGATCTCTGTCCCATTAGTTAGAGGGAGACCCAGACCCCATTGACTTTCTTAATAATGATTTTTTT
TGGAAGGGGAGCTAAAAGAATAAGTCCCAGCAACAATTTATTGCATTATGATCGCAGATCTAGGCTGTTA
ATTTAATTTGCGTGTTTGTATATAGTTATTTCCCAATCTTACTAATGAGGATTTTGAGTTCTAGAGCACT
GATTTTTTTTTTTTCTCCTTTAAACTTAAGGCTCCACCCACAGCCCATTCAGGACAGAATCAGGGTCTGA
GTTTCTCTTCTCAGCCTTGACAGACCCGAGTTGAAGAACCAGGTCTTCCTTTTATAAAGAGGGGTGAGAG
CCTCGAGATGATGGGTAGTCTCTGACTCTTAACTGGATCTGCTTCACACCTAGGTCCCGACATCTGTGGC
CCTGGCACCAAGAAGGTTCATGTCATCTTCAACTACAAGGGCAAGAACGTGCTGATCAACAAGGACATCC
GTTGCAAGGTGTGCCTGGGGGTGGTGGCAAATGGCTGTCATGGGGAGATTCAGAGGTCAGCCTCATTGGG
GGGTGGCCCCCGCTCACCTTCTTCCTTCTTCAGGATGATGAGTTTACACACCTGTACACACTGATTGTGC
GGCCAGACAACACCTATGAGGTGAAGATTGACAACAGCCAGGTGGAGTCCGGCTCCTTGGAAGACGATTG
GGACTTCCTGCCACCCAAGAAGATAAAGGATCCTGATGCTTCAAAACCGGAAGACTGGGATGAGCGGGCC
AAGATCGATGATCCCACAGACTCCAAGCCTGAGGTTGGTGTTTGGGCAGGGGCTCTGCTCTCCACATTGG
AGGGTGTGGAAGACATCTGGGCCAACTCTGATCTCTTCATCTACCCCCCAGGACTGGGACAAGCCCGAGC
ATATCCCTGACCCTGATGCTAAGAAGCCCGAGGACTGGGATGAAGAGATGGACGGAGAGTGGGAACCCCC
AGTGATTCAGAACCCTGAGTACAAGGTGAGTTTGGGGCTCTGAGCAGGGCTGGGGCTCACAGTGGGGAGT
GCACCAACCTTACTCACCCTTCGGTTTCCTTCTCCCTTCTGCAGGGTGAGTGGAAGCCCCGGCAGATCGA
CAACCCAGATTACAAGGGCACTTGGATCCACCCAGAAATTGACAACCCCGAGTATTCTCCCGATCCCAGT
ATCTATGCCTATGATAACTTTGGCGTGCTGGGCCTGGACCTCTGGCAGGTGAGACTTGGAGGAAAAAGGA
GGATCCCTGGGGTACCTCAAGTGCATAAGATCACCCAAGAGGAAAGGGACAGGGTAGGCACCCCAGGTGA
GTCTGACTCAAAAATGGTACTTCTTGTAAACAGTACTTCCTGGTCTGTCCCTGTGAAGTCCTCACAGCAA
CCCCTTTAAGGTTATACTTGCTGTGCACCAAGTACTTCCCCAAGTACTTTTATGCAAATCAACTTCTTTA
CCCCCAAAGACCTAGAAGGTGGTCAGGTAACCCAGTTAGTTAGCTGGGGCTGGGCACAGTGGCTCACCCT
TACAATCACGGTACTTTGGGAGGCTGAGACAGAGGATTGCTTGAGGCCAGGAGTTACACAACTCAACCTA
GCTTGGCAACACAGCGAGGAGACCCTATCTCTACAAAAAAAATTTTTTTTTTTGAGACAGAGTTTCACTC
TTGTTGCTGAGGCTGGAGTGCAATGGCACGATCTCAGCTCACTGCGCCCTCCGTCTCCTGGTTTCAAGCG
ATTCTCCTGCCTCAGCCTCCGGAGTAGCTGGGATTACAGGCATGTGCTACTATGGATGCCAGGCTAATTT
TTTTTTTTTTTTTTTTTTTTGAGACCGTGCCTTGCTCTGTCGCCCAGGCTGGAGTGCAGTGGTGTGATCT
CTGCTCACTGCAAGCTCCGCACGACCCCCCAGGTTCACTCCATTCTTCTGCCTCAGGGTCCCGAGTAACT
GGGACTACAGGCACCCCCCACCATGCCTGGCTAATTTTTTTGTATTTTTTTTTTTAGTACAGACATGGTT
TCACCGTGTTAGCCAGGATGGTCTCCATCTCCTGACCTCATGAACCACCCACCTTGGCCTCCCAAAGTGC
TGGGATTACAGGCGTGAGCCACCTCACCCAGCCTTTTTGTAGAGACAGGGCTTCATGTTGCCCAGGTTGG
TCTCGAACTCCTGGCCTCAGGTCATCTGCCCGCCTCGGCCTCCCAAAGTGCTGGGATTACAAGGGTTAGC
CACCATGCCTAGCCTCTACAAAAACTTTAAAAATTGGCGAGATGTCATGCATACCTGTAGTCCCAACTAC
CAAGGAAGAAGGATGATCACTTGAGCCTGGGGCATCGAGGCTGCAGTGAGCCATGATTATGTCACTGCAC
TCCAGCCTCGGTGACAGAGTGAGACCCTCTCAAAAAAAGTTGGGACTTGGCCGGACACAGTGGCTCACAC
CTGTAATCCCAGCACTTTGGGAGGCCAAGGCGGGTGGATCACAAGGTCAGGAGATGGAGACCATCCTGGC
TAACATGGTGAATGAAACCCCATCTCTAGTAAAAATACAAAAAATTTGCCAGGTGTGGTGGTGGGCGCCT
GTAGTCCCAGCTACTCGGGAGGCTGAGGCAAAAGGATGACGTGAACCCGGGAGGCGGAGCTTGCAGTGAG
CTGAGATCATGCCATTGCACTCCAGCCTGGGTGATAGCGAGACTCTGTCCCAAAAAAAAAAAAAAATGCT
GGGACTGAATTTTTGTCTGTTTTGGTCACTGAAATACCTTCTGTGCCCAAGACAGTTCTGGCATGTAGTA
GGTACCTGAAAAATACCTGAATAAGAGAGTGAGAAACAAGAAACAGGTGCAGAGAACTGAAGTCAGTGGC
CCAAGGTCATGGGGGTAGGAAACCACAAAGCTGGGGTTTGAACCTGGGCAGTACAGCACCTGAGTCTCTC
CATCTTTTTTTTTTTTTTTTTTTAAGACAGAGTCTTGCTCTGTCACCCAGGTTGGAGTGCAGTGGCTTGA
TCTCGGCTCACTGCAGCCTCTGCCTTCCAGGTTCAAGTGATTCTCATGCCTCATCCTCTCGAGCAGCTGG
AATTACAGGCATGCGCCACGACGCTGGGCTTTTTTTTTTTTGAGATGGAATTTCACTCTTGTTGCCCAGG
CTGGAGTGCAATGATGCAATCTCGGCGGCTCACCACAACCTCTGCATCCCAGATTCAAGCGATTCTCCTG
CCTCGGCCTCCTGAGTAGCTGGGATTACAGGGATGCGCCATCACAGACCCCGGGCTAATTTTTTTTAGTA
GAGACAGAGTTTCACTATGTTGCCCAGGTTGGTCTCGAACTCCTGGCCTCAAGTGATCCGTTCGCCATGA
CCTCCCAAAGTGCTGGGATTACAGGCATGAGCCCGTCCCGTCCCTGGCTGTCTCTCCATCTTTCCATCTT
TTTTTTTTTTTTTTTTTTTTTTGGAGATGGAGTCTCACTCTGTCACCCAGGCTGGAGTGCAGTGGCACGA
TCTTGGCTCACTGCAAGCTCCGCCTCCTGGGTTCACATCATTCTCCTGTCTCAGCCTCCCAAATAGCTGG
GACTACAGGCACTTGCCACCACGCCTGGCTGATTTTTTGTATTTTTAGTAGAGACGGGGTTTCACCGTGT
TAGCCAGGGTGGTCTCGATCTCCTGACCTCGTGATCCGCCCACCTTGGCCTCTGGGCGAGGATTACAGGC
GTGATCCACCTCACCTGGCCTCTCCATCTTTTTAACTGCAGTGTCAGCGGTGTTCCTTGTCTTCTCTGCA
GATGCAGGCAGCAGAATATAGTGGTTATAGGAACACAGGTGGAAACCCTGTCCAAAGCAAGGGCTATCGG
GTATCACCTCTGACCATCCTTCCCATTCATCCTCCAGGTCAAGTCTGGCACCATCTTTGACAACTTCCTC
ATCACCAACGATGAGGCATACGCTGAGGAGTTTGGCAACGAGACGTGGGGCGTAACAAAGGTGAGGCCTG
GTCCTGGTCCTGATGTCGGGGGCGGGCAGGGCTGGCAGGGGGCAAGGCCCTGAGGTGTGTGCTCTGCCTG
CAGGCAGCAGAGAAACAAATGAAGGACAAACAGGACGAGGAGCAGAGGCTTAAGGAGGAGGAAGAAGACA
AGAAACGCAAAGAGGAGGAGGAGGCAGAGGACAAGGAGGATGATGAGGACAAAGATGAGGATGAGGAGGA
TGAGGAGGACAAGGAGGAAGATGAGGAGGAAGATGTCCCCGGCCAGGCCAAGGACGAGCTGTAGAGAGGC
CTGCCTCCAGGGCTGGACTGAGGCCTGAGCGCTCCTGCCGCAGAGCTGGCCGCGCCAAATAATGTCTCTG
TGAGACTCGAGAACTTTCATTTTTTTCCAGGCTGGTTCGGATTTGGGGTGGATTTTGGTTTTGTTCCCCT
CCTCCACTCTCCCCCACCCCCTCCCCGCCCTTTTTTTTTTTTTTTTTTAAACTGGTATTTTATCTTTGAT
TCTCCTTCAGCCCTCACCCCTGGTTCTCATCTTTCTTGATCAACATCTTTTCTTGCCTCTGTCCCCTTCT
CTCATCTCTTAGCTCCCCTCCAACCTGGGGGGCAGTGGTGTGGAGAAGCCACAGGCCTGAGATTTCATCT
GCTCTCCTTCCTGGAGCCCAGAGGAGGGCAGCAGAAGGGGGTGGTGTCTCCAACCCCCCAGCACTGAGGA
AGAACGGGGCTCTTCTCATTTCACCCCTCCCTTTCTCCCCTGCCCCCAGGACTGGGCCACTTCTGGGTGG
GGCAGTGGGTCCCAGATTGGCTCACACTGAGAATGTAAGAACTACAAACAAAATTTCTATTAAATTAAAT
TTTGTGTCTCC

By “Heat shock cognate 71 kDa (HSP7C)” is meant a polypeptide or fragment thereof having at least about 85% amino acid identity to NCBI Accession No. P11142, as shown in Table 2, and having activity as a repressor of transcriptional activation and a chaperone, as well as a possible scaffolding activity during spliceosome assembly.

TABLE 2
HSP7C Polypeptide (P11142) (SEQ ID NO: 3)

| | |
|---|---|
| 1 | MSKGPAVGID LGTTYSCVGV FQHGKVEIIA NDQGNRTTPS |
| | YVAFTDTERL |
| 51 | IGDAAKNQVA MNPTNTVFDA KRLIGRRFDD AVVQSDMKHW |
| | PFMVVNDAGR |
| 101 | PKVQVEYKGE TKSFYPEEVS SMVLTKMKEI AEAYLGKTVT |
| | NAVVTVPAYF |
| 151 | NDSQRQATKD AGTIAGLNVL RIINEPTAAA IAYGLDKKVG |
| | AERNVLIFDL |
| 201 | GGGTFDVSIL TIEDGIFEVK STAGDTHLGG EDFDNRMVNH |
| | FIAEFKRKHK |
| 251 | KDISENKRAV RRLRTACERA KRTLSSSTQA SIEIDSLYEG |
| | IDFYTSITRA |
| 301 | RFEELNADLF RGTLDPVEKA LRDAKLDKSQ IHDIVLVGGS |
| | TRIPKIQKLL |
| 351 | QDFFNGKELN KSINPDEAVA YGAAVQAAIL SGDKSENVQD |
| | LLLLDVTPLS |
| 401 | LGIETAGGVM TVLIKRNTTI PTKQTQTFTT YSDNQPGVLI |
| | QVYEGERAMT |
| 451 | KDNNLLGKFE LTGIPPAPRG VPQIEVTFDI DANGILNVSA |
| | VDKSTGKENK |
| 501 | ITITNDKGRL SKEDIERMVQ EAEKYKAEDE KQRDKVSSKN |
| | SLESYAFNMK |
| 551 | ATVEDEKLQG KINDEDKQKI LDKCNEIINW LDKNQTAEKE |
| | EFEHQQKELE |
| 601 | KVCNPIITKL YQSAGGMPGG MPGGFPGGGA PPSGGASSGP |
| | TIEEVD |

By “HSP7C nucleic acid molecule” is meant a polynucleotide encoding an HSP7C polypeptide. An exemplary HSP7C nucleic acid molecule is provided at NCBI Accession No. NC_{—}000011.9, and is also shown below.

HSP7C Nucleic Acid Sequence (NC_000011.9)
(SEQ ID NO: 4)
CCTTCTGGAAGGTTCTAAGATAGGGTATAAGAGGCAGGGTGGCGGGCGGAAACCGGTCTCATTGAACTCG
CCTGCAGCTCTTGGGTTTTTTGTGGCTTCCTTCGTTATTGGAGCCAGGCCTACACCCCAGGTAAAACCTC
TGCTCAAGAGTTGGGTTGTGGGTCTGGGAGCGTGCAGCCTCCACACAGGCCTGTTGGGCTTGCTGAGGCT
TGGGGGTTCTGAGAATCTCGTCGAGGCGAGTGTGCGGCTCCTTCTACCGGCTTAAAGGGCCTCAGTTTTC
GGTGGGATGGCAGCGGTATTTGGTTGCAGCCGGCAGGACGGAAATGTAGGGAGTGGGCCGCAGTGGCCCC
AGGGGAGGCTGGGAGACGCCCGGCGGCCGCGTGGCGGGGGAGGGTTGCTGCATCGGTTTGCCTGGCGCGC
GGGGAAGTGGAGCCAGCGTTTTCTTTCACCCAGTTCCCTGCTTAGTCCAGTCCCACCGTGGTTCTTCAGA
GCTGTTCTTGGCGTGCTTCCAGTATGGGGGTACATTCCGGAGTAGTTAAAAGCCCGTTGACTCCCGGGGG
CACTGGCACCTGGCGAGGGAGGGGAACAGACAGTGCTCAGTTCGGGGTAAGACCACGTGTTGAGCAACGC
CCCACGCCGTCTGGGTAGATGGGTCCTTCATCTAGGGCGTGCTCTGCTGCGGTTGGCACGGCAACCTGGA
CTGCAGCACTAGTTCTGGACCTCGCGCGTGCTTAGACAGGAGGTGATGGGCACTATTACCTCTTGGCAGT
GGCCATACGTTTTTCCTGGTTAAGTGTTCTGTTAAGGGATGAGGGAAATATTTTGATTAATTGAATTTTT
AAACCAGATTTTTCTTTTTTTCAGCAACCATGTCCAAGGGACCTGCAGTTGGTATTGATCTTGGCACCAC
CTACTCTTGTGTGGGTGTTTTCCAGCACGGAAAAGTCGAGATAATTGCCAATGATCAGGGAAACCGAACC
ACTCCAAGCTATGTCGCCTTTACGGACACTGAACGGTTGATCGGTGATGCCGCAAAGAATCAAGTTGCAA
TGAACCCCACCAACACAGTTTTTGGTGAGTTCCTAATTTTAAATGACAGAACAAATATAACAGGGCTAGG
AAGCACAAAAGTTTATGAAACGTGAGGAGGGAACTTTTTGATTTTAGAAAAACTGAGCTGAGAGACTTGT
TATCAAGTCTGTTATAAAACAGGTTGTAGAAACCTTTCAGGCTGAAATCTGGATAACGTAGGAGGTTGAA
GTTTGAACCTTTGCTACCTATATGGTAGTTGAATTCACCTACCTATGAACTGTTAGGTATTTGAGTAATC
ATGGACTTGAGTTTTATCAGAAGAGCTATGAAATTGAAAGTGTTTTCATTTGACACCTTTTACAGATGCC
AAACGTCTGATTGGACGCAGATTTGATGATGCTGTTGTCCAGTCTGATATGAAACATTGGCCCTTTATGG
TGGTGAATGATGCTGGCAGGCCCAAGGTCCAAGTAGAATACAAGGGAGAGACCAAAAGCTTCTATCCAGA
GGAGGTGTCTTCTATGGTTCTGACAAAGATGAAGGAAATTGCAGAAGCCTACCTTGGGAAGGTGAGGTTG
GTTTTTCAGTATGGGGTGCATTCCGGAGTAGTTAAAAGCCCGATGACTCCCGGGGGCACTGGCACCTGGC
GAGGGAGGGGAACAGATGGGGCTCAGCTCAGGGTTAAGACCACGTGCCCAACAGTGCCCTAGGCTCTCTA
GGTAGATGGGTCTGTCAACACCAGAAACCAGTGAATCTTGACAATTACACAGTAATTTACATTTTGGTGG
GGGGGGTGCTCCAGCTGTTCTTTCACCAGCATTAATCCATTTGCTGGAGTTTGCATATATGTAAGTATAA
TAGTTACCAATCTGTGGTCTTTTCCTTATTCCTAGACTGTTACCAATGCTGTGGTCACAGTGCCAGCTTA
CTTTAATGACTCTCAGCGTCAGGCTACCAAAGATGCTGGAACTATTGCTGGTCTCAATGTACTTAGAATT
ATTAATGAGCCAACTGCTGCTGCTATTGCTTACGGCTTAGACAAAAAGGTATGTACCATTTGTGATGCAA
GTTCGGATTATTTTAAGATTAATTTGATCCATCGTAAATTTAAATGAGATTGTTTTTAACGGCAGGTTGG
AGCAGAAAGAAACGTGCTCATCTTTGACCTGGGAGGTGGCACTTTTGATGTGTCAATCCTCACTATTGAG
GATGGAATCTTTGAGGTCAAGTCTACAGCTGGAGACACCCACTTGGGTGGAGAAGATTTTGACAACCGAA
TGGTCAACCATTTTATTGCTGAGTTTAAGCGCAAGCATAAGAAGGACATCAGTGAGAACAAGAGAGCTGT
AAGACGCCTCCGTACTGCTTGTGAACGTGCTAAGCGTACCCTCTCTTCCAGCACCCAGGCCAGTATTGAG
ATCGATTCTCTCTATGAAGGAATCGACTTCTATACCTCCATTACCCGTGCCCGATTTGAAGAACTGAATG
CTGACCTGTTCCGTGGCACCCTGGACCCAGTAGAGAAAGCCCTTCGAGATGCCAAACTAGACAAGTCACA
GATTCATGATATTGTCCTGGTTGGTGGTTCTACTCGTATCCCCAAGATTCAGAAGCTTCTCCAAGACTTC
TTCAATGGAAAAGAACTGAATAAGAGCATCAACCCTGATGAAGCTGTTGCTTATGGTGCAGGTAACAATG
GTATCTCAATTAACCCTAAAGGCAGGCAGGCCCAAGGTGACTCGCTGTGATGAGTGATTGTTAAACATTC
GTAGTTTCCACCAAAAGCTTGGCTAATGATGGCAACACCTTCCTTGGATGTCTGAGCGAGTGATAGTTAA
AACAGGAGCTATGTACTGGGTTTTCTTTTAACTTCTTTTAACGTTAACTTTTTGTTTGCTAGCTGTCCAG
GCAGCCATCTTGTCTGGAGACAAGTCTGAGAATGTTCAAGATTTGCTGCTCTTGGATGTCACTCCTCTTT
CCCTTGGTATTGAAACTGCTGGTGGAGTCATGACTGTCCTCATCAAGCGTAATACCACCATTCCTACCAA
GCAGACACAGACCTTCACTACCTATTCTGACAACCAGCCTGGTGTGCTTATTCAGGTATGTTTCTGTACT
TCTCTTGTTTGGCTTACTGATAACAGATAAAGGGAAGTCTTGACTGACTCGCTATGATGATGGATTCCAA
AACCATTCGTAGTTTCCACCAGAAAGTCTTATGTTGGCCAGTTCCTTCCTTGGATGTTTGAGCGACCATT
CTTCCTTAGCAGGACCCTAGCACTGTCACAGACCTGGAGTCCATTGTAGTAATTTGTTTTATTTCCTACC
AAGGTTTATGAAGGCGAGCGTGCCATGACAAAGGATAACAACCTGCTTGGCAAGTTTGAACTCACAGGCA
TACCTCCTGCACCCCGAGGTGTTCCTCAGATTGAAGTCACTTTTGACATTGATGCCAATGGTATACTCAA
TGTCTCTGCTGTGGACAAGAGTACGGGAAAAGAGAACAAGATTACTATCACTAATGACAAGGGTAAGGAG
GCACTGTCATCTGGTCTTGACAGGGATAATGGTATTTCAATTGAGTTACTGGTGCCTAAGGGCGTCTAGC
TAAGAGAAACTAGAGTTACACATACACAGGTAATTTAAGGCTTTTACTTAGAGTTAATTTCTTTCCTAGG
CCGTTTGAGCAAGGAAGACATTGAACGTATGGTCCAGGAAGCTGAGAAGTACAAAGCTGAAGATGAGAAG
CAGAGGGACAAGGTGTCATCCAAGAATTCACTTGAGTCCTATGCCTTCAACATGAAAGCAACTGTTGAAG
ATGAGAAACTTCAAGGCAAGATTAACGATGAGGACAAACAGAAGATTCTGGACAAGTGTAATGAAATTAT
CAACTGGCTTGATAAGAATCAGGTTTGTGTTTTTTTTTTTTTTTTTTCCTCCCCCACTCAATGGAGGGGA
AGGGGATGGTAAACCAAGCTTGAGCTGGATTTCAGTGTAGGGTCACAATGATGAATGGTCCAAAACATTC
GCGGTTTCCACCAGAATTCAAGGTGTTGGCAACTACCTTCCTTGGATGTCTGAGTGACCCAAGATGTTAA
GGAAGAATAAGGCCCTATTTTAATGTTGGTAGTGGCCCTCTTGTAAGAGTTTGCGCCAGACTTTTAGTAT
CAGATTGCGTCAGGGAGAAAGAAGGGTTATTAACATTAAAAGAACTTGCAGTAATTCCTTTTTCTCTTCC
TCAGACTGCTGAGAAGGAAGAATTTGAACATCAACAGAAAGAGCTGGAGAAAGTTTGCAACCCCATCATC
ACCAAGCTGTACCAGAGTGCAGGAGGCATGCCAGGAGGAATGCCTGGGGGATTTCCTGGTGGTGGAGCTC
CTCCCTCTGGTGGTGCTTCCTCAGGGCCCACCATTGAAGAGGTTGATTAAGCCAACCAAGTGTAGATGTA
GCATTGTTCCACACATTTAAAACATTTGAAGGACCTAAATTCGTAGCAAATTCTGTGGCAGTTTTAAAAA
GTTAAGCTGCTATAGTAAGTTACTGGGCATTCTCAATACTTGAATATGGAACATATGCACAGGGGAAGGA
AATAACATTGCACTTTATAAACACTGTATTGTAAGTGGAAAATGCAATGTCTTAAATAAAACTATTTAAA
ATTGGCACCATA

By “Thioredoxin (THIO)” is meant a polypeptide or fragment thereof having at least about 85% amino acid identity to NCBI Accession No. P10599, as shown in Table 3, and having redox reaction activity.

TABLE 3
THIO Polypeptide (P10599) (SEQ ID NO: 5)

| | |
|---|---|
| 1 | MVKQIESKTA FQEALDAAGD KLVVVDFSAT WCGPCKMIKP |
| | FFHSLSEKYS |
| 51 | NVIFLEVDVD DCQDVASECE VKCMPTFQFF KKGQKVGEFS |
| | GANKEKLEAT |
| 101 | INELV |

By “Thioredoxin nucleic acid molecule” is meant a polynucleotide encoding a THIO polypeptide. An exemplary THIO nucleic acid molecule is provided at NCBI Accession No. NC_{—}000009.11, and is also shown below.

THIO Nucleic Acid Sequence (NC_000009.11)
(SEQ ID NO: 6)
CTCGCAGGCTCCAGGGGCGGGGCGTGGCCGGGGCGCAGCGACGGGCGCGGAGGTCCGGCCGGGCGCGCGC
GCCCCCGCCACACGCACGCCGGGCGTGCCAGTTTATAAAGGGAGAGAGCAAGCAGCGAGTCTTGAAGCTC
TGTTTGGTGCTTTGGATCCATTTCCATCGGTCCTTACAGCCGCTCGTCAGACTCCAGCAGCCAAGATGGT
GAAGCAGATCGAGAGCAAGGTACGCGCTACCGGGGAAGGCCAGGGTGCCGGCGCCGCGCGCGGCCTCTGT
AACTGGGGAAGGCGGTGGCGGGAGGTGGGGAAGGCGGTGGCGGGAGGTGCGGAGGCCGCCCCTCCGCATC
GCCAGGGGAAAGGGACGCGGCGTCTCGGCCTGGGACTGCGGGAAGCAGCGGCCTGGGCGCGCCCGAGGCG
GTGGAGCCTGCCCTGGAGGAAGGGAGGAGAAGGACGAGGGTCCCCTGGAGGGCGGAGTGGCGGTGCCCAG
CGTTTCTCGCACCCTGTTCCTCGGGGGATTGCACGCACGCGGGGAGCGTCCGGGGGATGTGAGAGCGCAG
ACAGCGTGAGGAGTCCCCACGCTGCGCCTCCTGCACCCTCCCGTCCGGGCAGCCCCGACTGGAGGAAGAT
GAGGGAATGGAAGGGGTCCGCCCTTGGCCCCCCATCTGTATCCAGATTCAGGCCCCAGGCAAGGATAGGG
AGGGCCCTTGCAGAAGGCACGGGTCGGTGGCCGCCGCTGCCTTTCCGTATGTGAAGTGATCCACCCGCAG
CGGGGGTAGTGATCTCCCTTTGGGAGCGGGTCTAGGCCGGAGACCCCCGCCTGCCTCCACCCATGCCCGA
CCCCAAAGGTGACGCGTGCTGTATCCGCACTAAGGGGGCGGATTGCGGCTGGAGACCCCCTGGCACGTGC
AGGTCTGTCCAGGAGGCCCGAGGGCCCCAGGTGACCGCGAGGAAGTGAGGTCCGGGCCGCGCCCACGGGA
CTCCTGTGGCGCAGGGCGCGTTTCCGGCAGCAGTGGCTTTGGAATGACTGAGTCCCCAAGGTTGGGCCCG
GGGGCCTCGGCTGCCCTGCCCGTCCATGATTCACCCTCAGTCGGTGGGTTTTGCTGGAGCCAGGGTTCCT
CCTGGGAGCAGCCGCGCCCTGCTGCCTGCTCGCCGACCTATCGGTATCCCGATCGTTGTTTTGTCCTCTT
AAAAATGCCCAAGGCGAAACAGCCTTCCCATGTTTGAAAGTTATTGCAAGCCTAAAACCTTGTAGACTGG
GAAACCCAGAGCCTAACGCGCAGTGTCTAGTCCAATGTAGCCACTCCAGAAATATTTGTTAAATGCAGCG
TCAGAAAAGTGAGTGGAGGAAATTGATACTGCTCGAACGGTAGAAGACCCCTCGCCAGCGCCTACCCTGC
GATTACCCCTCCCTACCTGCGGGAAGCAGAGGAGGGCGGGTCCTCGCCCGCCTCGGGTGCCCTGACCTGT
TTGGTGCCGGGTGGGCTTCGGAAACAGAAGTGTGTCTGCAATGTGTCCCCGATCCTTTTGTTCCTTTGAT
TATTATTGACTCTCAGTGTTTTTTCCTCATATGTTGATTGCCACTGTCATCTTTTATCTTCCTCTCAATC
AGTTTTTTCTTAGTGGGATTCTCATTTTAGCAGCCCTCATGTGTTGAAAAGATCCTTAGTAGTGAATTGT
CTTTCATATACTTTTTTTCCAAGCACCTATTGTGTGACAAATTATTAATCCATTCCTGGGGAAGGGAGTG
GGGCTGGGATTCTGTTCTCCAGGGTCTGGCAACCTCAGTATAACCCAACTGCTAAGAACCCCCTCCACTG
AGCCAGAAGACCTTTGAGTGGTCTATGTTAGTTGTCCCAAAATCCAGACACTACAAACAAAGTTGATTAG
GATTTCTGGAGCACACAGTTTAGTCCTCCCAGTTGTCAGAGCATGTCAGAGCACCTTCCTCCTCTACCAG
TGACAAAGGTGTACAAGGGTGACAGGAACTTTAAAAAAAGCACTACAGCCTGGGGCCCAAAGGCCCTGAT
AATCAATTAATCCTCAAAATAACAATCCAAAGTCATTGATCGAAAGTTACACTAATTTGATTGTTATTTG
TCTGTTAGTTTGTTTTTCGAGATGGAGTTTTGCCCTTGTTGCCCTGGCTGGAGTACAGTGGCGCGATCTC
GGCCCACTGCAACCTCCACCTCCTGAGTTCAAGCGATTCTCTTGCCTCAGCCTCCTGCACAGCTGGGATT
ACAGGCATGCGCTGCCAAAATGCCCAGTAATTTTGTATTTTTAGTAGAGATGGGGTTTCACCATGTTGGT
CAGGTTTGTCTCGAACTCCTGACTTCAGGTGATCCACCAACCTCAGGCTCCCAAAGTGCTAGGATTACAG
GCGTGAACCACCACGCCCAGCCTGTTATTTGTAAATGTTGAATACATGTTACATTTTCATCCTAATGGGC
TAAATTTGCACCATTTGCCATTCAGAACAATTCTGTTTCTGAGGTACTCTGTTGGTGCTTTAGGGCCAAC
TGGGATCTATTTCAGAGAGGAATGGAATAATTGACTGTAAATGTGATGAGGAAGAAATAAACACTTTTAA
AAAAAATGACACCTACCATTTATTGAACTCCCATCTACAAGGCACTTGGCTAAGTACTTCAGAAACCACT
CACACTTATTACCCTCAGAGTAGGTATGTTGAGGCAACGAGATCTTAGACTCTTGCTCCTATTTACCCCA
ACTACACTGTTCTGCTTCCCCCAGATTATTGGTGTCAGTGATGGAGACATTTATTAATCCTGTTAGTTTC
TGGGAGCTAGAAATTGTGATTTCTTCTTAGTAATACAATCTTGAATAATTTTCAAGCTGATACCCGTTTA
GAAGTATCAGAAGAGAATTTGTACATGAAGCCTGCACATACGTGGGGTGTAACTCATGTTCAGTTAGGCT
AAAAGTTATTGTTGCGTGCCTCTTTTCAGAATTTTAGGTACTTGTGCTTAAATTTGATTCAGAACTGTTT
TGGAAAAGCCTTGAGTATGTTTGAAATACCTTCCCTCTTGAAAGTAATCTCAAGTTTTTAATAAGGGTTA
ATCATGTTAAAAAAACAAAAATGTCTATTCAACCAGACATTGGCATTTCTTGACCTTTTTTCCTGTCTTA
CCTGGATCTTGCAATAAAGGATGCCTGGTTTAACTTTCTTGAAAATCACATTAGGGAAGGCTTTGAATGA
AATTGATCTGGAACAATAAGTGATGATTTGGAAAAACAATTGCTATACTTCTATGTACCCTGCTGCAGCT
CTCCCCATGTCTCCACCTCTAGAGGTGGGGTTCAGGGATTTGCATAACTAAAAAATTTATGAAAGTGTTG
TCCTACCTTTCTCAGGAACACCATTTGTGAATTATTTTCCCAAAAACGAGGTAGAAATTAGAAATCTAGA
GAAGTAACTATTAGTACATGAGGTCATATTAGTGTTTTCTTGTTGGGTTTTTTTTTGTTTATTTGGTTTT
TTATCTTATGGTTTTTTATTTATTTGATTTCTTTCTTTACGAGACCTCTTGTGGCGGTGGGGGGCGGGGA
ATGTTCATTTTTTTTTAAACCTATTTGACCAGCATTGTTTCCTTGAAGAAAACCTAGATTTCAGATACAG
ATGTTTATGTTTTGATTTATCTTAATTGCTCTGGTTTGGTTTTTGGGTTTGGTCAGCACTAACGTACTAA
TGTGGTTAAAATGAGTCCTTTGTTTTGGGAGGCCAAGGCGGGTGGATCACTTCAGGTCAGGAGTTCAAGA
CCAGCCTGGTCAACATGGCGAAACTCTGTCTCTACTAAAAATACAAAAATTAGCTGGGACTGGTGGCAGA
GGCTTGTAATCCCAGCTACTCAGGAGGCTGAGGCAAGAGAATCACTTGAACCCCGGAGGCAAAGGTTACT
GTGAGCCGAGATCAGGCCTTTGCACTCCAGCCTGGGCAACAAGTGAAACTCCGTCTCAAAAACAAAACAA
AACAAAAATGAGTCCTTGGTAACTAGAATATTCGGTTCCCAGGGTTACAGTATCTAGATAGTAAATAATT
CAGGGAAGTTAGTGGTAAGAGATTTCTTGATCATTTCTACTGAGAATTTTATTTAACAAGCATTCCTTAT
GAAAAATAATATCTATGAAAAATTTCCTTCATGAGGAACGAAAACTTTCATTTAATGAATGACAAGGGTA
TAGTTTTAAAATAAAGGGCAAAAATCAAAGGTTGGTAAACGTGTGATCTCAGCTCTGGAAACCCCATTAT
GCTTATGTCAACGGTGATGTCTGAGTGTTGAGGTTTGGGAAAGGTGAGTTTCCTTGACTTTTCAAAAAAT
TTTAGATTTTCGTATGGTCCACCATAGACAAATGAGTTTAATCAAAAGTCATAGCTTTTTTTTTTTTTTT
TTTTTTTGCGACAGAGTCTCCGTCTATTGCCCAGGATGGAGTGCAGTGGCACAATTTTGGCTCACTGCAA
CCTCCGCCTCCTGAGTTCAAGCTATTCTCCTGCCTCAGCCTCCTGAGTGGCTGGGACTACAGGCATATGC
CACCACGCCCAGCTAGTTTCTGTATTTTTAGTAGAGACAGCATTTCACCATATTGGCCAGGCTGGTCTCG
AACTCCTGACCTAGTGATCCGCCCACCTCGGCCTCCCAAAGTGCTGAGATTACAGGTGTGAGCCACCATG
CCCAGCCAACTTTTATCTTTAAGTAACTTGTGATGTTTCAATTGCAAAATCCTATGCCTTTGTGACTTCA
AGTGACCCCTTTCATAATCCATAAGTGTTTAATGAATGTCTACCATATACCTAGCCTTGACATGGAAACA
TTTTTAATACAAATGTCTATTTTTATTTTCCTTTTGTTTGGTGTAGAGAAAAAATAGCCAGTTCACAATA
TTTTATAAAATAGTTATGAAGAGAATGTCAGTATACTCTACACATATCTTGTTTCATCTTATCAAGTAAC
ACTACCAACAATGTATAGAATTTCTTCAAACTGAGTTTTATTTGGCTTGTTTGGGGATTTTTTTTTTTTT
TTTTTTTTTTTTTTGGCTAAAAAGTAGGTCCTGAAAGGAGGACCTCCAGAATGTGCTTTGTGTCATTGTG
TCGAGTCTTTCTTTTGAAGGTTTAATATTTAACTATTTATTTAATATAAGCTTTTCTTTTGCTGTTAGAC
TGCTTTTCAGGAAGCCTTGGACGCTGCAGGTGATAAACTTGTAGTAGTTGACTTCTCAGCCACGTGGTGT
GGGCCTTGCAAAATGATCAAGCCTTTCTTTCATGTGAGTATTAAACAATGTCTGCTTTGTAAGAGATTTG
TGTTTTTTGAGTTGGTGGTCACAGTGGTAGGAAAGAAAGACAGTTAAAGGATTTTGGTTTCGGTGGGGGG
ATTTCTTTGGCTGGATCTTTGGTCTAAAAGTAGTAGTATAACAAATAATTTAGGTTTGATACATGTAGCC
CATTGAAAACAAATTTTAGAAGTTAATTTTGTCTTAAATAGTTCTTTTTTTCCCCACATTGAAACATGGG
CCTTATTTGAAATCCCAGCCTCAGAATTTGATATGCCAAGCTGTTTTATACTAAGAAAAATTTGATTTAG
AGAAAATTTATGTCTCTTAGATCTATGTCTCCAAAGATCTAAATTTTTGGATCTTTAATTAGTCTCTACT
TTTATTAAGTTTCCATTTAAGAAGCTTGGGTATGTTGATTGCCATTACCTAGTTCTAAATCTTTTTGGAT
TTTTCATTTTAAATTTTCCAGTCCCTCTCTGAAAAGTATTCCAACGTGATATTCCTTGAAGTAGATGTGG
ATGACTGTCAGGTATGTAGCTGGAAATATGAGATACTGCTGAGCTTTTCACATTGGCCTTTTTCTCTGAA
TTGCACAGTGCTTTTTTCCATAAATATGTCAAATAATTCTAGAACTGTAATCCTATCTAAAAAGTTCTAT
CTCAGAAGAGCAGGCAAGTTAGGAGCTTAATCCTAGCTATCGGGAGCTGTATATCACATCCTAAAGTAAA
CAAAAATAAATGAGTGAGACTTCTGAATCTTATCGGCCACCCACCTTTCTAAAACCCTACATTCTACTTT
ACACTCTGAGATGTGCAATAAATGGAGATTGAATTTAGCTATGATCATTACATCCATAGGCTTGATGGAG
TCACCAAATTATGAGACCGCTTGTAGGGCTCTTTGTGAACTTGCAGTAGCATGAGAACCTGCATTTGCAA
GCCTATTCTAGTCTTGGTTGATTTTAGTCAATTAGAAACCACAAATGTTTTAACAAATAAACACCAAGGT
ACCTGAGAGAATAATTTGGAAGAAATTCCAGGGTTGGTTGTATTTAACAAATACTTGTTTTGCACTAGGT
ATATACCAGGCACTGTTCTGGGTGGTTTTTAAGTATCAGTTCATTTAATCCTGAGTGCTGTTATCATCCC
CATTTTATAGATGAGAAAACTGAAACACAGAGGTTGTTCATGAAGTTTCAGTGAGTATGTGGCTGAACTA
GGATTTAAAATGAAGTGGTCTGGCTTCCCAGCCCTTGACCTTAAGCACTACCCATCGGAGGATGCTCTGT
CTTGTGGGTGTAGATCGGGTGCTTAGCACATGACCACAGACCTAGGAGAGCGGGTTGAGGAGGTATCACT
TCGGGGCCCTTTACAGATATGTGAGCATTTTCACTTAGCCCTAGTGGAGAAGGAAAGGCGATGGGGGAAG
GGTGCAGTGTGGCAACAGAGGCGCTGGACCTGGCTTCCAGTCCTGGCTCACTAGCATCTGCTTAGGCCAG
TCACTCCTCTTCCTTGAGCCTTAAGACCTGCCCCATCAACCTCCCAAGGTTGCTTATTCATTGAGCAAAC
ATGGAATATCCAATAAAGGGTGAAGGGTCACTTAAAACAGGCATATGGCAGTGCTCTCTAAACATGGGAG
GGCGCAACAACCCCAGATTGTGTATTCTTAGCCAGTTTTTGACTCTGTGCCTTGGGCAACCCCTGCCTTG
GCTTGTGCTGTCTTCTCCATCTGGCCTGTCCTTTCCTTTCCTACCTGACTAACTCCTTGTCATGAGCTTC
ACCCCTTCTCCACTTACCGCCTTGTGTGCCCTAAGTACCCAGTGAATCTTGGCAATTATTATAATGATCT
TTATGTCTGTCCTTTACCATTAGTCTCAGTAGATTCCTAGGATCAGAGACCCTGTCTTAATTCACGTTGG
TTGCCCCTTCACCTAGCACACCTGCCTTGCATGTAGTATAGGTGTGGAATGAATGAATGATGAATGTGAT
ATGGTTGTTAAGTTACTATTCTAGATGTGTCCCAGAGTTGTTTTTTTTTTTTTAAAAAGAGTGTAATTGC
ATTTTTGTGAAAAATCCTTATCCCTTGTTTTAATCAAACTTAGTCTTATTAAGGTCAATTTAGCTAGGGG
AAAATTGCACCTGGAATAGAGAAATTCTAACTGCCACTGATCCTATCAGATAGCAACTTGATTTTTTTTT
TTTTTTTTTTTTTTTTTTTTTTTTTGAGATGGAGTTCACTCTGTCACCTGGGGTGGAGTGCAGTGGCATG
ATCTCGGCTCACTGCAACCTCTGCCTCCCGGGTTTAAGCAATCCTCTGTCTCAGCCTCCTGGGTAGCTGG
GATTACAGGCATGCACCACCATGCCTGGCTAATTTTGTATTTTTACAAAATTAAAACCCCAGTAGAGACG
GGGTTTCACCATGTTGGTCAGGCTGGTCTCGAACTCCTGACTTCAGGTGATCCACCCACCTCGGCCTCCC
AAAGTGCTGGGATTATCCACCACGCCCGGCCTTGATTTTTATTTGAAAGCAATAATAGGTGCCAGATGCC
ATGATAAGCCCTTTGCATGCACTATGTCATTTAATCCTCACGATAACTATACGAGTATTTTTTATTAGCA
CCCTCATTGAACAGGTAATGGCACTGCAGCACAGAAGGTAAAGTCAGTCTCTTGAGGCAGACCAATGCAC
CATACTGTACTGAGGACAGGTCTTCTTACTGCCTTTAGGAAGTACAGTCATGCATCACTTAATGATGAGG
ATACTTTCTGAGAAATGTTAGGCAATTTTGTTGTGCACACATAGAGTGTACTTACACAACCTAGATGGCA
TAGCCTCCTGTACACCTAGGCTATGTGGTAAAGCCTGTTGCTCCTAGGCCACAAACAGGTAAGGCATGTT
ACTGTACTGAATACTGTAGGCAGTTATAACACAACAGTAAGTATTTGTGTATCTAAACATAGAAAAGGTA
TGGTGAAAACATGATATGAAAGATTAAAAAATGGTATGCCTTTATAAGGTACTTGCCATAAACGGGACTT
GCAGGACTGAGAGGTGCTCTGGGCGAGTCAGTGAGTGGGTGGTGAGTGAATGTGAAGGCCTAGAACCTGT
AGACGTTATAAACACTGTATGCTTACAAATTTTATTTTTAAAATTTCTTTTTTCAGCAATAAATTTATTG
TAACTTTTTTACTTTATAGTTTTTTTATTTTTTTAACTCTTTAACTCTTGTAATAACACTTAGCTTAAAA
CACGAATGCATTGTACAGCTCTACAAAAATATTTTCTTTATATCCTTAGTCTATAAGCTTTTTTTAAAAA
GACTTTTTAAACTTTTTGTTACAAACTAAGATTCAAACACATACATTAGCCTAGACCAACACAGGGTCAG
GATCATCAGTATCACTGTCTCCCATCCCCACATCTTGTCTCACAGAAAGGTCTTCAGGGACAGTAACATG
CATGGACCTTTCATCTCCTATGATAACAGTGCCTTCTCCTGGAATGCTTCCTGAAGGGCCTGCCTGAGCC
TGTTGTATAGTAACTGTCTTTTTAAAAAAATAAGTAGGAGTACACTCTAAATTAATAATGAAATTAAAGT
AAATACAAAAACCAGTAACGTGGGTGTTTATTATCAAGTAGTATATACTGTCCATAATTGTAGTGATATG
CTTTTTTAAGTGAAAGCAAGTTTATTAAGAAAGTAAAGGAACAAAAGAATGGCTATTCCGCAGGTAAAGC
AGTCTGTAGTGGTATACTTTGTATGTAATTGCAGCGCAGATTTGTTTGCACCAGCTAATGCGATGGGCTA
TGACATTAACCCATCACTAGGTGAGAGGAATTTTTCAACTTCATTATAATCTTATGGGACCACCACATAT
ATGCAATCTGTTGTCGATCTAAATGTTATATGGTGCATTACTATAGGTGTGCAAAGCACTCGAGGACTTC
CGTATGACAGAGCTCCTCCTTCATGTCTGCTTGGTGCACCCTGATCACCCTGAATGTATCTTTTTTTTTT
TTTTTTTTGAGACAGAGTCTCACTCTGTCACCCAGGCTGGAGTGCAGTGGTACCATCTCTGCTCACTGCA
ACCTCCACCTCCCGGGTTCAAGCGATTCTTCTGCCTCAGCCTCCTGAGTAGCTGGGACTACAGGCAGCCG
CTACCACACCAGGCTAAATTTCAACTTTTTAGTAGAGACAGCATTTTGCCATGTTGGCCAGGCTGGTCTC
GAACTCCTCACCTCAAGTGATCCACCCGTCTTGGCCTCCCAAAGTGCTGGGATTAGAGGCATGAGCTACC
GTGCCTGGCCTGAAAGTGTCTTTTAAAACCTTGAAGTGACCCTCTGACAAACTGAGGAACTTTAACTTTG
CCTCCATAGATTGATAGAAAAGTATGAGTAGTAGCCCTTTTGAAAATGATAGACCAACCTTATTTCTCTG
ACAGCCAACAGGGTTATGATACTTATTTTATAAATGGTAACCTCCCTCTGACCCTTACTTGGAGTGAGTT
TTCAATAGTATGCATTCAATAAACATTCACCATTTTTATTCCAGCCATTACTGTCCTTGTGCCTCTTACT
GGAACCTGTACTTTCATGCTCAGCAGGTGTCCAGCATTAAAAGAAAAAGTAAAGATTACCTAGAAAGAAC
TCCTCAACAGTAGTGCCACCCACCATCCTAGAGGTCGTCATAGTGTTTGTAGCTGGCCTTTCTTCCCCTT
GAGAATTCTCCGTTGGTTTCCGTGATTTGGTTATCAACAGTCCTGCCTGCTCGCTTGCTGTCCTGTGTAG
CTTTTGCTGCTTAGGTGCTGAGTGGTTCTATATTTCTTTCCCAGTCCTCTTTTGAGTGCCTGGCTGACAT
TTTCAATCTCTATTGGGCTCCAAACCAAACCAGTTTCGTGGTATTGTCCTCCAAACCTTGCCCTCTTATA
GCATGAACAATGTGTTGAGCATGGGGTATTATAAGAGTTCTCATTTAGCATTCCACAGTTGAGGAATGTG
TGTTACTTCAATTACCTTTGAGCTGTAGAAAAATCTTTAGCTGTGGTAACAGCCACTTCTAGGAGAGGAG
AAAATACGGATCAACTAGCCCAATTTGCGATGTTAGGAATTTGTCGATTTTCTTAGTAGGATGGCTTTCA
AAGGTTAGAGCATCAGAGTCACCTGAAGCCCGACTTTAACTGTAATGGTTTAAGATGGGGTTGATGGGGA
AACTTGTAGTACCCCTCAGGTAATTCTGATACTGCAGCAAGGTTTGAGAATTCACAAAGTCTTTTTATTT
TTCCTCCCGAGATAGTCTCATTCTGTCGCCCAGGCTGGAATACAGTGGCATGATCTCAGCTCACTGCAAC
CTCCGCCTCCCAGGTTCAAGCAATTGTCCTGTCTCAACCTCCTGAGTAGCTGGGATGACAGATGTGTGCC
ACCACACCTGGCTAATTTTTGTATTTTTAGTAGAGATGGGGTTTCTCCGTGTTAGCCAGGCTAGTCTCGA
ACTCCTGACCTCAGGTGACCCACCGGCCTTGGCCTCACAAATCAGTTTTTAATTAAAAATAAGCAGGAGG
CTGAGTGTGGTGGCTCACACCTGTAATCCTAGCACTTTGGGAGCCCGAGGAAGGTGGATCACTTGAGCTC
ATGAGTTTGAGACCAGCCTGGGCAACATGGAGAGACCTTGTCCCTATAAAAAAAAAAAAAAAAAAATATA
TATATATATATATATATATATATATATATATAGTGTGTGTGTGTGTGTATATATATACACACACACACAC
AAAATTAGCCAGGTGTGGTGGCGTGTGCCTGTAGTCCCAGCTACTGGGGAGGCTGAGGAGGGAGGATGGC
TTGAGTCTGGGAAGTGGAGATTGCAGTGAGCTGAGACTATGCCCCTGCATTCCATCCAGCCTGGGTGACA
CAGCCAGACCCTGTCTCAAAAATAATAATAATCAGTAAACCCAGTGTGGGGTTATTCCTTTAGATTACTA
TTATTTTGTTCTTGAACAATTGATTTTTATTTTTTTAGACTTTTTAGCCTTTATATAATCATTCTGTGTA
CTCTGCCTTCATAATAAAACTGGAAAAATTATGAGCAAGAAATAAGAGGTACTAGTTCTGAGGAATAGTT
AAGATTATCATACTGAGTCCAATTGTAGCAGAATTTTTTGTTGCTTCTTTGTATGATACTTAAAATAGTT
GAAAATTTGATTGGATTAAAGAGCATATTGGATCGCTGGAGTATCTGATGCTAGTAACATTCTGAACATT
CTGCCTGTTAATGTGCCCGTCAAAGGAAGTAAATATTAATAAAACTTCTTCATTGAGAATATAACCGGTT
TGGCTTTTGTAATGCCATTATATTCATTATATTAATTTTCATATGCTGAAAAATGTCCTCATGCGGAAAT
GTGGGGTACATGACAGGGAAAAGTTTCTGGTTTTGGATTACTTCTGTCAAAGCTCAGTACTCGCAGTCTT
GTATTTAATCCTCTCCCTTTGCTACTTTCCCTACCAGGATGTTGCTTCAGAGTGTGAAGTCAAATGCATG
CCAACATTCCAGTTTTTTAAGAAGGGACAAAAGGTACGTACATCTGACCTTTAAAACTCTAACTGGGCAA
TAGGAAACCCAGTATAAGTGAATAAATCACTGGAGTGATGTTCCCTTTAAAGATTGAGGCATATCACCAA
GTTCTGCTTTTAAGAATTTTTAAATATGCCAAAATTCATTGGCTTAAGTACATAATGTGACAGCTAACTG
AAAATCAATCTTTCCTAGAACTAGTCCTATTTATATCATAAAGCACATAGAATTTCTTAGACTTGGGCAG
TTCATTTGTTGTTAAGTATTGTGTAAAAGAAAATTTGTACTTGAGCCTTTTGACTTTTCTCTTGATATTT
TTTCTTTGTTTATAACTTAAATGAACTGTATGTTATTCAGGGAAGTTTACTTTAAATAAGATTATACTTC
TTTTTCCCTCCACCCCTATTCTTCCTTCATTCTATGCTGAATACATATTTATACATATGTATATATATAC
ATATGTATATGTATATATATAAATACATATTTATACATATTTTATGTATAAAACAGTGCTACAGTGCTAC
GTCTAATGTCAATTCAATATTCTCTTAACAGGTGGGTGAATTTTCTGGAGCCAATAAGGAAAAGCTTGAA
GCCACCATTAATGAATTAGTCTAATCATGTTTTCTGAAAATATAACCAGCCATTGGCTATTTAAAACTTG
TAATTTTTTTAATTTACAAAAATATAAAATATGAAGACATAAACCCAGTTGCCATCTGCGTGACAATAAA
ACATTAATGCTAACACTTTTTAAAACCGTCTCATGTCTGAATAGCTTTCAAAATAAATGTGAAATGGTCA
TTTAATGTATTTTCCTATATTCTCAATCACTTTTTAGTAACCTTGTAGGCCACTGATTATTTTAAGATTT
TAAAAATTATTATTGCTACCTTAATGTATTGCTACAAAAATCTCTTGTTGGGGGCAATGCAGGTAATAAA
GTAGTATGTTGTTATTTGT

By “Tropomyosin alpha-3 chain (TPM3)” is meant a polypeptide or fragment thereof having at least about 85% amino acid identity to NCBI Accession No. P06753, as shown in Table 4, and having actin filament binding activity in muscle and non-muscle cells that may, among other activities, play a role in stabilizing cytoskeleton actin filament activity in non-muscle cells.

TABLE 4
TPM3 Polypeptide (P06753) (SEQ ID NO: 7)

| | |
|---|---|
| 1 | MEAIKKKMQM LKLDKENALD RAEQAEAEQK QAEERSKQLE |
| | DELAAMQKKL |
| 51 | KGTEDELDKY SEALKDAQEK LELAEKKAAD AEAEVASLNR |
| | RIQLVEEELD |
| 101 | RAQERLATAL QKLEEAEKAA DESERGMKVI ENRALKDEEK |
| | MELQEIQLKE |
| 151 | AKHIAEEADR KYEEVARKLV IIEGDLERTE ERAELAESKC |
| | SELEEELKNV |
| 201 | TNNLKSLEAQ AEKYSQKEDK YEEEIKILTD KLKEAETRAE |
| | FAERSVAKLE |
| 251 | KTIDDLEDEL YAQKLKYKAI SEELDHALND MTSI |

By “Tropomyosin alpha-3 chain nucleic acid molecule” is meant a polynucleotide encoding a TPM3 polypeptide. An exemplary TPM3 nucleic acid molecule is provided at NCBI Accession No. NC_{—}000001.10, and is also shown below.

TPM3 Nucleic Acid Sequence (NC_000001.10)
(SEQ ID NO: 8)
AGATAAAGACTCAAGTCTGGGGACCTCCTGGTCACTCAGGCAGCAGCCCCTTCTTTCTTGCCCCAGTCTC
CAGTTCTCCAGTGTTCACAGGTGAGCCTACCAACAGCCACTGCTCATGATGGAGGCCATCAAGAAAAAGA
TGCAGATGCTGAAGTTAGACAAGGAGAATGCTCTGGATCGGGCAGAGCAAGCTGAAGCTGAGCAGAAGCA
GGCAGAAGAAAGAAGTAAACAGGTAGGCACTGGCATTGATCTCTCTCATCTGCTAGTGAACAAGACTGTG
AAATGGAAGGGAGTTTTCATGGGAAAGACAGTGACTACTGGTGTCACCTCCTTTTTGGGGGTGGGATGGA
CTCCCCAGCCTCACTGGAGGAGCAGAGGATATCTAACATATCTGTCTCAGACTGCCTTTGTGACCTTGGG
AAAGTCAGTGAGCCACAGTATGTCTGCTGAATTTAAGTAGAAATGGACCATGTTTTGGAAGAACCCTTGA
CTTCTAGAGGAAAAGGTAAGAGTTCTAGAGGGAAAGAGCATCTCTTTTCACTCAAATAGCATCACGGGCA
TCTACACTTTAGATATTTATCATCCAGGGGTCTTGGATGAAAACCCAGGATGGCTTGGTCATCTTAGGAT
CAGAGATTACAGGGTCACAGGCCAAGCTACGGCATAACTCCTGAGGCAGTGGGCTGGAAGTGGCTGGGGA
GAAAGTGTGTCTCACTAGGGATGGTAACATGGAGGTTACACTCAGGGCTCCAAGAGCCCAGGGTCCAACC
CTGCATTGTTGTGTTTGTGTGTTTTTGTGTGTGACTCTCTGGGCTCCTATAGCTGGAGGATGAGCTGGCA
GCCATGCAGAAGAAGCTGAAAGGGACAGAGGATGAGCTGGACAAGTATTCTGAAGCTTTGAAGGATGCCC
AGGAGAAGCTGGAACTGGCAGAGAAGAAGGCTGCTGATGTGAGTATTAGAGAGATGGAGGATGGGTTTAA
TCTACACATATAGATCTTTATGTTAATGTATGACGAAATTCCAAACACACTTGTGGGTTCATGGAACACA
GACATATATACTCACATACATTTTTGCATATAACCACATGCTGGTATATTCTCATAATGGCATGTATTTT
CTTTCTTAAAGACATCTCCAACATGTTAAAAGTTTTTTTTTAATACATGTAAAGGTCTCATTTATCTCCT
ATCACCAACCCAGGAATTTAATTATCTTTCCCAGGACCTGGACAAAGAGAGGGGACTCTAAGTTCAGAAT
GCTGTCACTTATCCTACCTCCTCCCCCATGCCACATTCTTAGCTTTTATGTCCAAGTCTTGGCATGTGGA
TATGTGTATTAATAGGGAACCTCTCATCTCTCAACTGTCCAACAAAACCAGCTCGGATTTCACAATGGGA
AATGGAGGTCACACTGACCCTGGCCATATATGGGTTTATTTATCTTGGCCAATGCAGGCATTTGCCTTTA
TGCCATGGAGATGCCATATGAAGCATTGCTCCTAGGGCAGCAGAGAGATCCCTACAGTGACAGAGTATAA
TAGAAACTAAGAAAGAATCTTAGGGGGTAAAGGTATCACCACCAGACTTCACTCTATGACCGTGAGTAAT
TACTCAGCTTGTCTAGGGCTGAATTTTCCCATTCTCAAATAAAATAAATTCTCTTTTCCATCTCATATTA
TCCCATCATCATCACCTACTTTCCTTCATCTTTAACCTTGAAGTTTGCTGCAAAGATAAATGAGAGAGTA
GATTGAAAAGTTTTTTAGAAGAAAAGTACCATAGGAATCTTACCAACAACAACAGAAATGTCCCAGTATT
GGTGGAAACAAGTTTCTCAGTGAACTGAGTTAATAGGAAAAAAGGAGCATGTAATATTTAAAATCATATT
TGGGCTGGGTGCGGTGGCTCACGCCTGTAATCCCAGCACTTTGGAAGGCCGAGGCGGGTGGATCACCTGA
GGTCAAGAGTTTGAGACCAGCCTGGCCAACATGGTGAAACCCTGTCTCTAGTAAAAATACAAAAATTAGC
CAGGTGTGGTGGCGGGTGCCTGTAATCCCAGCTACTCAGGAGGCTGAGGCAGGAGAATCGCTTGAACCCA
GGAGGCGGAGGCTGCATGGAGCCGAGATCGTACCACTGCACTCTAGCCTGGGCAACAGAGAGAGACTCTG
TCTCAAAAAATAAGTCAATAAAATAAAATCACATTTGGTTTTGGAAAATAGCTAGAAGGACAGACAGAGT
GAACCATTCATAAATGCTTTCGGATCCTCATATTTGGTCTCTCTGCCATCCGGTTTCTTTCCTTTTCTTT
TCCTTTTTTTTTTTTTTTTTTTTTTGAGACAGGGTTTCACTCTTGTTGCCCAGGCTGTAATAAAATGGTA
CTATCTCGGCTCACTGCCATCTCCACCTCCCAGGTTGAAGCGATTCTCCTATCTCAGCCTCCTGAGTAAC
TGGGATTACAGGCACGTGCCACCATGCCTGGCTGGCTAATTTTCATATTTTTACTAGAGACGGGGTTTCA
TCATATTGGTCAGGCTTGTCTTGAACTCCTGACCTCAGGTGATCCGCCTGCCTCGGCCTCCCAAAGTGCT
GGGATTACAGGCGTGAGCCACCCCGCCCAGCCAGGTTTCTTTTTATTGTTGTTGTTGAGATGGAGTCTCG
CTCTGTTGCCCAGGCTGGAGTGCAGTGGCACAATCTAGGCTCACTGCAAGCTGTGCCTCCTGGATTCAAG
CCATTCTCCTGCCTCAGCCTTCCGAGTAGCTGGGATTGCAGGTGTGTGCCACCATACCCAACTAATTTTT
GTATTTTTTTTTTAGTAAAGATGGGGTTTCGCCATGTTGGCCATGCTGGTCTCGAACTCCTAACCTCAAG
TGATCCGCCTGCCTCAGTGTCCCAAAGTACTGGGATTACAGGCGTGAGCCACTGCGCCCAGCCTAAGCCA
CTGCGCCTGGGCTTTTTTTTTTTTTTTTTTTTTTTTTTTTTTTTTTTTTTTTTTTTTTTTTTGAGACAGA
GTCTTGTTCTATCACCAAGGCTGGAATGCAGTGGCAAGATCTCGACTCACTGCAACCTCCACCTCCCGGG
TTCAAGTGATTCTCCTGCCTCAGTCTCCTGAGTAGCTGGGATTACAGGCAGCTGCCACCATGCCCGGGTA
ATTTTTTATATTTTTAGTAGAGACAGTGTTTCACCATGTTGGCCAGGCTGGTCTCCAACTCCTGACCTCA
AGTGATCCACCCACCTTGGCCTCTCAAAATGCTGGGATTACAGATGTGAGCCACCATGCCTGGCCTCTGC
CATTAGGTTTCTGATCTGTTTTGTTTTCCACCACTTTCAGTGGCCAAGGAAAGTGGTAAGAATTGAATAT
ATTATGCCAGAGGTTAGAAACACAAACTAAATATACATCATTTAAACTCATTCTTCCAAAAGTTAAGGCC
AGCACAGGCTTTTTTTTTTTTTTTTTTTTGAGACGGAGTCTTGCTGTGTCACCCAGGCTGGAGTGCAGTG
GTACGATCTTGGCTCACTGCAAGCTCCGCCTCTTGGGTTCACGCCATTCTCCTGCCTCAGCCTCCTGAGT
AGCTGGGACTACAGGTGCCCGCCACCGCGCCCGGCTAATTTTTTGTATTTTTAGTAGAGATGGGGTTTCA
CCGTGTTAGCCAGGATGGTCTCGATCTCCTCACCTTGTGATCCGCCCGCCACGGCCTCCCAAAGTGCTGG
GATTACAGGTGTGAGCCACCGCGCCCGGCCAGGCCGCCACAAACTATTAATTCTCCCTTCTTGTGCCCAG
TGTGGTGGCTCAGACCTGTAATTCCAGCACTTTGCTTTGAGAGGCCAGGGCAGGAGAATCGCCTGAGGCC
AGGAGTTCAAGACCAGCCTGGGGAACATAGTGAGGCCCTATCTTTACAAAAAATTTAAAAATTAGCCAGG
TGTGGTGGCATGTGCCGGTAGTCCCGCGTACTCCAGAGGCTGAGATGGGAGGATCGCTTGAAGCCAGGAG
TTCATGCTTGCAGTGAGGTATGATAATGCTACTGCACTCCAGCCTAGGTGACAGAGCAAGACCCTGTCTC
TATTTAAAATGATAAAAATTCTCCCTTCTCAGAAAACTCAAAAAAACAAAACAAAACAAACAAACAAACA
ACCCTACCAAATCTACAGGTTACCCCTGGGCTTTGGGCAAGTGGATGGCTGGCTGGAGTTGTATAGCCTA
GGTTCCTGGTATATTTGGATGGATCTCTCTGTTTCTAACATATGGGCATGAAGATAGAAGTTTTCACAAA
CTAGCACAATGTCAGGTTAATAACAGTTTTGTGAGAAATTGGGTAACATCAATCTGGGTCTGCTCTTAGG
CTGAAACGGCTAGTATGTGGGCTTGAGGAGGTGTTTCCAGGAGCAAGAAAAAGAACAAGTCTAACCTGAG
ATCATGAGAAGGAGAATAACATAGAGGCTGTACGCAATACATCAAAATCATTAGGAATATAAAAGGATTT
CCTGGGGATGAATTCTGTCTATCTTAATCTGTTTTACACATGTACAGACATACACATACACACACATAGC
ACCCCTCACCTAGATGTGGTCTACTCAGTATATTCATTTGAAAGAAGTTTCTTCATATTTACATAGGAAT
CCACCAAAACCCATTTAAATATGTAAAGTGAGGGTTAAAAATACTCATTCAACCCCTCTCCCCTTTGTTA
GCTTTCCCAAGTCAGAAAGTACCCTCAGAGATATGCATAAGAACAAGAAACAGGAGAGGGAAGGTTGTGG
CCAGGAGCAAGCAAGAAGAAAGGTTTCTTTCCTCAAAGCTAGAGTCAGCAAACTACCACCTGTGGGTTAA
ATCTTGCAACAGCCTGGTTTTGTAAATAAAGTTTTATCGGAACACAGTCATGCTCATTCTTGTACATACT
GTCTACTGGCTTTCTTATCTCTCTGGCCACAAGACCCTAAAAGTTGGATGACCCCTGCCTCAAAGGGTCA
AAGAAGGACAGTGTTTATAGGAATTGGGAGAGGTGCTGAATAAGGACCAACAAGGTTAAAAGCAGAAAGC
AAAGGTAAGAGTCATCAGCAGCATAATATATGCATCTTTGGAGGGAGTATACTCTGTCCAATACAAAGGA
AGTTAACAGCTCAGCTTCTGTCCTTGAGGTTAATAAATAAGTAAACAGAGCATATTATCTTGAAGTAGAT
ATAAGCAGTAGAGAAGGAGGAGGGAGTCAATAACTACGTGTTTACACAAGCAGTTCTGGTTATCTGAGAA
GCCTTTCTGAAAAGTGCAGGATTTACTAGGACTGCTTGAATAAAGAAAGGGCAAGGGTATGGCAAGGCGA
GTCCAAACTTTTTTTTCCTCCCTAAATTTGTCTAACTTTGTTTTCTAAGTTGTGGTAAAATACGCATACA
TAAAACTTACCATTTTAACCATTTTTAAATGTCTAATTCAGTAGTAGCATATTTACATTGTTGTGCAACC
ACATCTAAGATCATTTTAATCCAAGATGAAATCAGATGCTGTTTAGGGAGAAACAAAGGGAAAGAGGAGT
GCATTGTTAGGACATAGAGTCATAACACCATTTTTTAGGGACAGGTGGCAGGAAGGATGGGGAGAGAAGG
GACAACGATGGACATATACACCTGTTGATATATTGTATTGCTGGTTACTAGGAACACAATAGCACTGGGC
CCCTTGCTGTCTATCCATTGTGCTTCTAAAGGCTATCATGAGTCAGTCCATCTGTTTGACTGCATTAAGA
GGAGATTATCTGCTCAAATCTACCAGTCTCAGAAATGCAGTGGTGCTCTCAATACCCTCTTGGTTTGGCT
AGGATGAGGTCTTCTGCTGCTTTACCAGGGCACAGGGAGGTACAATAGTGCCCCCTGCTGAGCAGCACCC
AGATGACAAAGTGTCACTTCTGCCACATCTTGATCAGACTTCTACAAAGCAAGTTAGAAAAATTCTTAGG
CCATCAGGGTAGAGTCTATTAAGAGTCAGAACCCTGATCTTCCTTCCTAATTTCTGGGGGAAAAATCAGT
GTGCAGGTTACTCTATTGAGAAAATTATTATCTATCCAAGTTGTTTCTTCTGAATAGTCTCATTGCTCGG
TATTTCCAAAAGGTGATAGGAAAACAGAGGCTCTGCAGGGAGCATTCTTAGAACTAGACATTGAGGGCAG
CAACTGTCTTGCCTACTATTCCTTTCAGGGATTAGGACTCTTCATTGTAAGTCCTGTTCCACATCCTAAT
TTTCTAGGAGAAAAGAGAGCTGTCCCAGAAGAAAAATTTATCACGATTACCTACTGGGAAGGTGGGAGGA
ATACAAAGAAATAAGAGGGAAGATAACGTTCTCAAGTCTCTTTACTATTGTGCTTCCAGAAATCCTGCAT
GGCAGTGCCTGTAGGGAAAGTTTCTTTTTTTGTTGTTTTTCTTTTTTTTTTTTTCAGACTGTGTCTCACT
CTGTCACCCAGGCTGGAGTGCAGTGGCGCGATCTCGGCTCACTGCAACTTCTGCCTTCTGGGTTCAAGCA
ATTCTCCTGCCTCAGCCTCCCGAGTGGCTGGGATTACAGGCGCCTGCTACTGCACCTGGCTAGTTTTTTG
TTTGTTTGTTTGTTTGCTTGTTTTTGTGGTTTTTAGTAGAGACAGGGTTTCACCATCTTGGCCAGGCTGG
TCTTGAACTCCTGACCTCGTGATCCACCTGCCTCAGCCTCCCAAAGTGCTGGGATTACAGGTGTGAGCCA
CTGCGCCTGGTCTGTTTTGCCATTTTTTTTTTTTTTTTTTTGATACAGAGTCTCACTCTGTCACCCGGGC
TGGAGTAGAGTGGCACCATCTCAGCTCACTGCAACCTCTGCCTCCTGGATTTAAGCAATTCTCTTGCCTC
AGCCTCCCTGAATAGCTGGAACTACAGGTGTGCACCACCACACCCGGCTAATTTTTGCATTTTTAGTAGA
GACGGGGTTTTGCCATGTTGGCCAGGCTGGTCTCAAACTCCTGACCTCAGGTGATCTGCCCGCCGTGGCC
TCCCAAAGTGCTGGGATTACAGGCATGAGCCACCACATCTGGCCAAAGGGAAGTTTCTAACTCATAAACA
GTTCCTTGGGGGTACTTGGAAGTACAGTGGCATTGAAATTGGTAAAAGTTGTAACAGAATGGAGGCTCAA
TTCTATAGTACAGTTTACAGGCACTCATGTCTGGCTGTTGCATGAAGGATCTAGTAACAGTCGTCTGTTT
TTTATTTTTTTGAGACAGGCTCTCAATCTGTCACCCAGACTGGAGGCAGTGATACAATCTTGGCTCACTG
CAACCTCTGCCTTCCCGGCTCAAGAAATCTTCCCACCTCAGCCTCCCAAGTAGCTGGGACTACAGACGTG
CTGCCACCAAGCCTGGCTAATTTTTTAATTGTTTGTAGAGACGAGGTCTCACTATACTGCCCAGGCTGGT
CTCAAACTCTGGGCTGAAGTGATCTTCTCGCGTTGGCCTCCCAAATGTTGGGATCACAGGCATGAGTCAC
CCCGCCCGGCTGACAGTGGTCTGTAATAGAGGATTTCTTCCTGATAAAATGGGGGCATATCTTTCTTACC
TACAAAAGGAATTGTAAGTGATTATTAAATTATTTATTTATTTTTAGAGATGAGGTCTTGCTATATTGCC
CAGGCTGGTTTTGAACTCCTGGGCTCAAGCCATCCTCCCACCTCAGCCTCCCAAAATCCTAGACTTACAG
ACATGAGCTACCACGCCCAGCTGATGATTAAATTGCCAAGTCCTGAATCATTCCAAGGTTTGGAGGGGCT
GGAAGGTTGGTTAATTTGGTGCATGATGACTCTCAAAAGTAACAGGGACCTGTAATCCCAGCACTTTGGG
AGGCCGAGGCCGGCAGATCACGAGGTCAGGAGATCGAGACCATCCTGGCTAACACGGTGAAACCCCGTCT
CTACTAAAACTACAAAAAAAAAATTAGCCGGGCGTGGTGGAGGGCGCTACTCGGGAGGCTGAGGCAGGAG
AATCGCTTGAACCCGGGAGGCGGAGCTTACAGTGAGCCGAGTTCGCACCACTGCACTCCAACCTGGGCGA
CAGAGGAGACTCCGTCCCAAAAAATAAAAAACAAAACAAAAATAAATAAAATAACAGGGAGCCTGACGGG
GTGGCGTGCACCTGTAGTCCCAGCTACACCGGAGGCTGAGGTAGGAGGCTCGCTTGAGCCCAGGAGTTCG
AAGCTGTAGTGAGTTGTGATCTCGCCACTGCCGCACTCCAGCAACAGAGCGAGATCTTGTCTCTTAAAAA
AAAAAAAAAAAAAAAAAAAAAGGAGAAAAGTCTGTTCTAAATAGAACACTTAAGTTCATACCATGCCAAA
TAGAATGAGAATTTGTGCCATCTTGCCCATCCCTTCCACTCCGACAGTTCCATTCATTGACTTCCAGGAG
ACGGGAGAGAGAACCTACCTACCTGAGCCGACTTTGAAAGGAGACTGAGTGGGGCTCTTTTGAACCTGAG
GCCCAGAAAGTCTTTTAGACTTTTCTTATAACTTGGTTTTGGAGTTTGTCGTGGGGTGAGTGGAAGCAGA
GACTTGTAGGATGTTTATATTGTGGAGCAAATACAGTGAGGGGTGACGGGTATCGGCATTGCGGGAGTGA
GGGGTCAATTGCGGGAGTGTAAGGTCAGCCGACTACCTGCAAAGGGGCCAACCATTGTGGGCAGCCAGCC
TGGCGGGAGAGGCTAGGGCACGAAGCGCCGCCCAGTTACCATGGTAACACTCCCTCGTCCCAACCCCGTC
CCAGTTTACTGGCCCAAGGTAAGGCCGGGAGCCGTACTCCAGGGCGACTGGGAGGATCCACTCCCTGCCG
CGGCTCTCCCTCCAGTGGACTGCCTTCCCGCCTGCCTGGCGGTGGGAGGGCGCGGCAGAGGGACGTCACA
TCCGGGCGGGTTGGTGAGTTCCGGTATTTCAGGGCGTAGCAGGCGGAAGTAAGGGTGAGAGGAGGCTGCA
ACGCCGAGCGGAGGAGGCAGGAACCGGAGCGCGAGCAGTAGCTGGGTGGGCACCATGGCTGGGATCACCA
CCATCGAGGCGGTGAAGCGCAAGATCCAGGTTCTGCAGCAGCAGGCAGATGATGCAGAGGAGCGAGCTGA
GCGCCTCCAGCGAGAAGTTGAGGGAGAAAGGCGGGCCCGGGAACAGGTACGGAGATGGTGAGGCTCATGA
GGGAGGGGCAGGCGCTGGAGTATCCGGCAAGCGGAATAAGGTCCCTTTTGCCGTGGAGCACGGTCCTCTG
AAGGCCGGCGGGCTCGGAGATGAAGTTGGGATCTCGGAGGATGGGTGGGGCTCAGCACCAGTAGGACACC
AAGCGGACGAGGGGGAAACAGTGCTCGGGCGAGCAGGCTTGACCTTTGAAGAAATTGAGCCTTACGGGGT
TTGGGGTGTGGCTTTCCTCCTCTTTTTTTCTCCACCTCATTCATTCAGTGCCTCCGATCCGGGCGCGGCC
GTGGGAGGGGCTCAGCGGCTTCTCCCTCCCCCACTTCCCCATGGAGGGTGGGAGAACTTTGCTCTGGCTT
GAGGTTTCCTGCCCAAGGGGTAAGAGTGGGTGGGTGCTTCTCTGTTCGGGGAGGGGCAGTACTGAACCTT
CTGTGAAATGGTGAGGGTTTTGGGGTGGGAACCGTCGGAAAGACCCCGGCCTGCCTGCTGGCAGCTTTTC
CGCTCCCTCCTCCTGCTCAGGAAATGAGGCAGCAGTTGGCTGAGAGGAAGCGCGGTGCCCTCCCTCCCTG
GAGGAGCTCGAGGCCACAGGACCGCCTCTCATCCTCTGGCCAGGGGCCCTTTCCTCTTACGGGGTGCTCT
TGCAGCTGCTACACCCACTCTCTGAATCTACGTGAAACGGCAGGGGAGGGGATAGTACTTCTCTCAACGT
TAGTGCAAATGTAGGAGCCAACCAAACTTGCCTGGCCCTACCCACAATGACCTGGAAGGCAGGAACTTCT
GTCAGATTCAGGCCTTTATTGCTGAAGTGTGATCAGTGCAAGCCGAACTGGTTAAGAACATTTCTTCCGA
GCAGAGCTGATAAGCGAGTGTATGGCAGAGAATCAGATGGTGTGCCAGTCTCAAGGTTTCTGATTGACTT
GGGCAATTTGGTGGAGACAATAATGAATTCCAGGTGACTCTAGTGGAATATTTGTTCAACTGAAAGTGGA
AACATAAAATGTTGGTGGAGTGAACAGTGATTCTAGTGATTTCTTTCTTCCTTTCTTTTTTTTGAGACAG
AGTCTCACTCTGTCGCTCAGGCTGGAGTGCAATGGCATGATCTCGGCTCATTGCAACCTCCGCCTCCCGG
GTTCAAGTGATTCTCCTGCCTCAGCCTCCTGAGTAGCTGGGATTACAGGTGCCCGCCACCAAGTCTGGCT
AATTTTTTGTATTTTTGGTAGAGACGGGGTTTCGCCATGTTGGCCAGGCTGGTCTCGAACTCCTGACCTC
AGGTGATCCACCCACCTCTGCCTCCCAAAGTGCTGGGATTACAGGTGTGATCCACCGTGCCCAGCCTATT
CTAGTGATTTCTTTAAACGTCTTTCTGTTTAAAAGTGGCTGATTAGTTTTAGTGTAAGCCATAAGATTTT
CCAAGGTCTAGGGTGCAGTCTAATAGATGGAGCTGATACGAGTTGAGGCTTTGTTTTGGAGGATCTTGAG
AGAGTAAAGTAACTGTTGCTAAAATTCAAGATCCAGTGAAAGTACTCATGTGGTTTATTCTTTGTTCTTT
GTTCTTTCCCTTGGTAAGTTTTTGTTTTTGTTTTTCTTTTTGGCTGCTCCTCTGGGAAATAACTGAGAAT
GTTTTTGGGAGGGGGAGGGGTGGATTTCAGAAAAGGATGGTAATTGGGGGACAGCTGCCCTCCAGTTTAA
ATACTTAAAGATTGTTGGAATACTCACTTATTTTTATAAGTGGTCATGGAGATGCTGGGATGGGAGCTTG
TTGTCTTCCTCAGACACCTTTTTCTAGGTAGATTTTTTTCCTCTGTTCAGTTTGCCAACAACTTCTTATT
ATCTGTGCAAGTGAGATGGACTGGCTGGGTGACTCTTTATGGCCAGTGGCACTTTCTCATCTCCTTTGTT
CTGGAGACCAGATAGCTTTAAAGGGGAAGACAGAGGAACTGGCCATGTGTTGAGCTGCAGAATCTTAGGC
TCAGTATTTAAATATGGACATTCTAGGAGTAGTTCTAAGGTGGGTGATTCCTGCCTCCCATCCCCCCTCT
TTTTTTTTTTAGATGGAGACTTGCTCTGTAGCCCAGCCTGGAATGCAGTTGCACGATCTCTGCTCACTGC
AACCTCCACCTCCCAGGCTCAAGTGATTCTCCTGCCTTAGCCTCCTGAGTGGCTGGGATTACAGGCATCC
ACCACCACACTCGGCGATTTTTTTTTATTATTTTATTTTTTTATTTTTAGTAGAGATGGGGTTTCACCAT
GTTGGCCAGGCTGGTTTCAAACTCCTGACCTCAAGTGATCTGCCTGCCTCAGCCTCCCAAAGTGCTGAGA
TTACAGGCATGAGCCACTGCGCCTGTCCTGGGGTGATGTTTCTTAAGCTACTTTCACTCAGAGCCTGCCA
AATTTGCTTATTGAGCTGAGGTCTAGTGAGGAAGTCCTCAGAATGGGGAAGTAGAGTTATAGTTAAATAT
TGATGAAATCATTTCTTCTACTCCCCAGCCTGAAGAAGCTTAATCTTTTGACTTTCTGCTGCTCTGCTGA
ACTTTGAACCCCTACTGAGGAGGAGACACCTGTGGGAAGGGAGGGGGGTTGCTTGGGCTACCTGTTATCC
TCTTAAGGCATTCTGGAATGTGGGTTTTGCAGACTCCTCCCTCCTCTGGAGCGGGAAACTGCACTCTCTG
ATGGTATCTCATGCTGTTAGTGGGATTGTCACTTAATTATTTTCCTGGTTACTTTGTGTGAACATCCAAG
CCACCCTTGTTGGGAAGCTAGATGAGTCTAAAACTGAGACACTGGTGTTCTAGGATCCATCTTTCTTTTC
TGCTCTCGGCCTGGAAATTGGTCTTTTATAATGTAGCACCTTGTCAAATTTACTTTCAGTTCTGTATTTC
CTTTTTACTTCTGTCTAAATGGAAACAGATGGCAACATAATGTTTAAATGCAGTCTTTTTCCTTATCTCT
TTTTTTTTGTGGTAGTGGTGATAAAAATACATGTAAAATTTACCATTTTAACCATTTATAAATGTACAGT
TCACCTTATCTGTTTTTAATAACTCTCTCCTAATTGTCCCTGATATTTTTCTCTTTTTAATTTTTTTCTA
GTCTAAAAAACTTAAGAGGCTGGGTGCGGTGGCTCACACCTGTAATCCCAGCCCTTTGGGAGGCCTAGGC
GGGTGGATCTGAGGTCCACCTGTGGATCTGATGAAACCCCATCTCTACTAAAAATACAAAAAATTAGCTG
GGCATGGTGGCGCATGCCTGTAATCCCAGCTACTTGGGAGGCTGAGGCACAAGAATTGCTTCAACCCTGG
AGGCGGAGGTGGCAGTGAACCAGGATTGGATCATGCCACTGTACTCCAGCCTGGGCGAGTATCGGCACTG
GGGGAGTGAGGGGTCAACTGCGGGAGTGTAAGGTCAGCACACTACCTCTGCCTCAAAAAGAAAAAAAAAA
GTAATAACAAAAACCTTAAGAGTACTACCGAGGGAGTGGGCTTTGATTCTGGCCTTAATCCTAAATTCCT
AGTCTCTGGAGAGGTTAGAATGCTTGACTGTTGTGTGGTTCTCTTCTATTCGTGGGCCTGGTGGACTAAG
CAACCTTTCTTTGGTTGCTTTGCTTTCCTTTCTCTTTCCCCAGTACTAGAACACACACTCACAGGGGAGA
GGCAGATAGCCCTTTACCCCTGCTTAGGCTCCGCCTTTGTTTACTGTGGGGCAGATAGCTCTACTCCCTT
CCAACTTGCCTCCCCTTTGGTGAAGTTTAAATTCCCCAAGGACACGGTATCTCCCTGGGTAATGCATTTG
TGAGCACAGTGAACTCAAATAGTCCTTTACTCACAGGGCTCAAGCCTAACTTTCTGATACTCTTTCATTT
GAATTTTATCTGTGGCTTTTATCTAATAAGAGAAACCCCAGTGTGGCAGAAAGCCAGATTTAGGCTAATC
CCCTTCTGTCCTTGGCCCTGTTCTGCCTGAGAGTACCATCACACTGTCAGTTTCTTGCCTGGAGGGGTTG
ACATATTTGGGAAATCTCTTTGGAGTGGAGGATAGCCGAAGGTGTTGTCAGCCATCTGGTAATGTGACTG
AGTTGCTGGGGCATTCTACTCTTTCCCCAATCCCTCACCCAGGATCTTTGCTTCCTGGGCAGTGGAGAAT
CCTATTATTACTTGAAGGGAAGAACCACAGAGTACTGTATATCCTCCACACTGGGCAGAGATAGGTCAGC
TGAAACACCAAAACCGCATCTGGATGGGCATGCTAAAACCTAAGATGATTAGGAAAGTGCAGAGCTTAAC
GTTTCCCAGGGCCACAGGTAGCCTGGTCTGCAAAAAGTAGGCCTGTTCAATCTCTTGGTACCCATTGGTA
TTATTTGACCTGAAAGGGACAGGCACTGTGCATAGACTCATGAGGGAAAAGTCAGCTTGTCTTCCTTGTT
CCTTTAAACAAAGTATCAGATCTTGATTCTGCAGGCTTTTGTTTCATTGTAAAGAGAACTGATCCTCTGT
GCTAGGAGAAAAGAGAAAGGCAAGGTCCCATTCCCTACCACTCCACCCTGGAGGGGGTCTCTGAAGCTTT
TTCTAGGACTCCCATCTCAGGTTCTGCTTTTGATACAGGCTTGTTCATGTGTGCATGCTCTTAGGAGGGC
ACCAAGATGTGTTTTCCCCCTCTCCCTTTTCAGCCTTGCTAAGAATTCTATTGTAAATGCTGATTTGTGG
GAGAAACTGCCTGAGCCAGCTTGCTGTCATGGCAACTGTACACCCCCGCCCCATAGCCTGTTATCTTCTA
CTCCAGTTTGGGTAACTACTGGTGAGCTGCATGATGTCTTACATTTCATGAGCTTCCTCTAAATCTAGTT
GTATTTTTCGTCTGAGGGGGGTAAGTCATTTTACTAGCAGTGTCTCCTAGGTCTTGCCTGATAGCCTGAC
TTGCTCTTGATCCATGGCCTTGTTGGGTTTGTGAGGAGAAATGTGTGTTGTTACAGAGAACATCTATTTT
TCTGGAAGAGGCAGAAGCAGAGAATACTTGTCTTTGTGGTGTTTTGAGTAGTGTAGTTAGAGGCACAAAT
GGTTTCTTAATGATTTCAGCTTATGTTGACTTCTCGATTTTCTTTGCCTGGTGGCTAGATTGTAGCCCCA
GCCCTCTGCTTCCCTTCCTAGTCTCTTCAGCTTCTCATGTTTGTATGTGGAAGTTTTTGTGCCAGTCCTG
ATGGTTAAAAAGGAGTCTTTTGGTCTATATTCTTCCTCATCGGAGCTAGTGGCCCAGGCTGTTTCTATAG
CTGCACCAACTCCTGCCTGCCAGGAAGAAATCAGGGTAATAGAATTCAGGCACAATCTGGGCTATAGAAG
AGAGATCGTGTTTACCAGGCACTTGGATAAGCATCTGAACTGGGTGGTGTGCGGGGCATATTAAATTCTT
GTATACTGTTGCAGTCAGGGTACCTCCCACCAGTAAAGGTAGCGGGGAGGCAGTCATGGAAAGAGGTTTT
CCTACCATTAGAATTTATCCATCCACCTGCAGGCTGCCTCCTTCTTTCCTTGTTTCTTAGAGAAAACCCA
TCCAGGATGGGTTTACTTGGACTCTAACCTATATACTTCATTGTGAGATGGAGGCCAGGGGGAAGATTGT
CGCTGTATTAGAGGGTGAAGCTAAGGGGATGGCTGGAGGAATAGAAAGGAGAAAATTCAGAAATTTGGAG
GAATGGTAGTGGGTGTGGTGGAAATGTTCTTTCTTTTTTGTAAAGCTATCAAAGCAGATAATTTTAGCCA
GAAGCAGCTCCTTGTAGCCAACTTAAAAAAGGAGTACGGGGGCACAAGTTGAAGGGCTGGTGAGTAGGCA
TCCCAACAATATTCCATTCTTTTGCTTTCCAGTCTGGAAGGCTATTCTGGAAGACATTCCCTTGGCTAGT
TATTCTTCTGATGTGTTAGGGTAAAGTGATCATAGGAATAACGGTTTGAGTTAAGCAGCAATTAATAGGT
AATCCAGGATTCCCTCTTTTTTTTTTTTTTTTTTTGAAATGGAGTTTCACCGTGTTGCCCAGGCTGGAGT
GTAGTGGCGCAATCTCAGCTCAGTACAACCTCTGCCTCCCGGGTTCAAGGGATTCTTCTGCCTCAGCCTC
CCGAGTAGCTGGGACTAGAGGTACGCGCCACCACACCTGGCTAATTTTTGTATTTTTTATTAGAGTCAGG
GTTTCACCATATTGGCCAGGCTGGTCTCAAATTCCTGACCTCATGATCCACCCGCCCCAGCCTCCCAGAA
TACTGGGATTACAGGCATGAGCCACCACACCCAGTCAGGATACCCTCTTTAAGAAACCTTGCTTTTTTTT
TTTTTTTTTTCCTGCTTTATGAGACACTTGAAAGGTTACTGAGGATGAGACATGCCTCCAAGCCAGAGCT
CAGGCAGAAGGAACCCTTCATTGACTTTCTTTTCTGACTCCAGTGTGTTGAGGAATTTGAACTGAGTCCA
GGAAGTGGGTGGTACTAACTCACTGGGCTGGGCAAGGAACTGAGTTTTAAAACACTCTTCCTGTGGAACA
TATGGGAGTTCGTTGATGGGAAAGGGAGGAAATATATATATATAAAGTTATGTCTGCTAGATCTTTTAAC
AGCTGAGCTAAGACAAGTTCTACCCTGCGAGACTCTGGCTTTTTCATGGTAGTCAACCCTGATCTTAATG
TCATAGTTATTTCTTCTTTTCTCCATGCCTCTGCTTCCCTTGTCCATTCTTTCCTTCAAATGCAGGCTGA
GGCTGAGGTGGCCTCCTTGAACCGTAGGATCCAGCTGGTTGAAGAAGAGCTGGACCGTGCTCAGGAGCGC
CTGGCCACTGCCCTGCAAAAGCTGGAAGAAGCTGAAAAAGCTGCTGATGAGAGTGAGAGGTGTGTAGGGA
AGCCTGATGGAGTGTGGATTTTAAAAGTTCATAATAGTTCTGATCAAGATTTCCTTATAGACCTGTTTTC
TAAATAGCAATTCTGGCTGCGTGTGATGGCTCACGCCTGTAATCCCAGCACTTTGGGACGCTGAGGCAGG
AGGGTGGCTCAAGCCCAGGAAGTTGGGGCTACAGTGAGCTCTGATTGCGCCACTGCACTCCTGGGTGACA
GAGAAAGACCCTGTGTCAATCAGTCAATAAAAATAAAATACTAACTCCATCACACAGGATCTCCTTTCTA
TTCTGCATATTCTGTCCCCTTGAAGACAGTAAGGGTAACTTAGATGTTGTGACCCTTTTGTAATTGGTAT
TTCCTTACAAATTGGCCTAATGATCCTTAAAATGGGATCACCAGTATGACTTCTACTTTCTGGATCATAA
GCTTCTATTTGTGAGATTAATTTCTCTAAAACAGAGGTTCTCTAAGTAAGGTCTCAGACCAGCAGCATCA
GCAATACTTGGGAACTTGGTAAAAATTCACATTCTCAGCTCCTATGCTGATCTGCTGAATCAGAAACTTT
GGATGTGGGGCCTGGCAATCTGTTTTTAGTAAGCCTTCTCAACGCTGGTTGCATTATTAGAATCATAGGT
AGGGGTGAGGGGGGAAATCACTGGGGAATTTAAAACTGGATGCTTAGATCTCATCCCAGTTCCATTAGAA
TCAGTTGCAAGAGGGGTCTGGGCATTGATGTTTTTAAAGTTCTCCAGCTAATTTTTTTTTTTTTTTTTTT
TTTGAGACAGAGTCTCGCTCTATAGCCCAGGCTGGAGTGCAGTGGCGCAATCTGGGCTCACTGCAAGCTC
CGCCTCCCGGGTTCACGCCATTCTCCTGCCTCAGCCTCCCAAGTAGCTGGGACTACAGGTGTCCGCCACC
ACGCCCGGCTAATTTTTTGTATTTTTAGTAGAGACAGGGTTTCACCGTATTAGCCAGGATGGTCTTGATC
TCCTGCCTTTGTGATCCTCCCGCCTCGGCCTCCCAGTGTGCTGGGATTACAGGCGTGAGCCACTGCGCCC
GGCCTTCCCCAGCTAATTTTAATGTGTTCCTAGGGTTGAGAACCATGGCCCTTGAAACAAATTATAACTC
ATGTTTTTGAAAATAGCCAGTTGTCTCCTTGCCACATGTGAGTTAAACTAGAACAGAGGTGCTTGACAAC
TAAGGGTTTTCAGAATAAATAGTGCCTTTCTAAGGGTACGTTACTACTGTGCAGCTGTGAAAATGAAAGA
GGCAGCTATGTGTATACTATTAGAGTAGCATGCAAATTATAGAAAGTGAGGGCCGGGCGCAGTGGCTCAC
ACCTGTAATCCCAGCACTTTGGGAGCCTGAGGTGGACGATCACTTGAGGCCAGGAGTTCAAGACCACCCC
AGCCAACACAGCAAAATCCAGTCTCTGGTAAAAGTACAAAAATTAGCCAGGCGTGGTGGGGCGCGCCTGT
AATCCCAGCTACTCAGGAGGCTAAGGCACGAGAAGCACTGGAACCCAAGAGGCGGAGGTTGCAGTGAGCC
TCCCTCTTTTTTTTGGTCTCAAAAAAAAAAAAAGAAAGAAAGTGAGGATAGCTGCATGCTGAATACCGGG
GGGAAAAAAAAGAGTGTGGAAAACAAGTTGCAGAACCGTCTAGTATATTATTACTTTGGGGAAGCTGGGA
TGGGATTTTTGTGTGTATATATATATATATATATATATATATATATATATATATACACATATATTTAAAT
AATATTTATTTATTTTTTTGAGACAGAGTCTCACTCTATCCCCTAGGCTGGAGTGCAGTGGCACAATCTC
GGCTCACTGCAACCTCCACCTCCCGGGTTCAAGCCATTCTCATGCTTCAGCTTCCCTCAAGTAGCTGGGA
TTACAGGCGCCTGCCACCACGCCTGACTAATTTTTGTATTTCTAGTACAGACAGGGTTTTGCCATGTTGA
CCAGGCTGGTCTCAAACTCCTGACCTCAGGTGATCTGCCGGCTTCGGCCTCCCAAAGTGCTGGGATTACA
GGCGTGAGCCACCACGCCCAGCTGTATATATCCATATTTTTATTTAATATTTTAGTATTTAATTTCTTAA
TAAGTTTTTTTGGGGGTGGTGGAGGAGACGGTGTCTCACTCTGTCGCCCAGGCTGGAGTGCAGCAGTGCG
ATCTTGGCTTACTGCAACCTCTGCCTCCCGGGTTCAAGTGATTATCTTGCCTCAGTAGATGGGATTACAG
GTGCTTGCCACCACACCTGGCTAATTTTTGTATTTTTAGTAGGGACAGGGTTTCACCATGTTTGCCAGCT
GGTCTCGAACTCCTGACCTCAAGTGATCCACCTGACTTGACCTCTCAAAGTACTGGGATTACAGGCATGA
GCCACTGCGCTTGGCCAGGATGTATTACCTTTATAACAAAAAGTTTTTTTTTTTTTTTTGAGACGGAGTT
TCGCTCTTATTGCCCAGGCTGGAGTGCAATGGCTCGATCTCGGCTCACCGCAACCTCCACCTACTGAGTT
CAAGTGATTCTCCTGCCTGAGCCTCCCAAGTAGCTGGAATTACAGGCATGTGCCACTACGCCCAGCTAAT
TTTGTATTTTTAGTAGAGACGGGGTTTCTCCATGTTGGTCAGGCTGGTCTTGAACTCCCGACCTCAGGTG
ATCTGCCCGCCTCAGCCTCCCAAAGTGCTGGGGTTATAGGCGTGAGCCACTGTGCCCAGCCATAACAAAA
AATTTTAAATGAAATAATTAAACTAAGGTGTGGGAGTAGTAACATGGAATTTCCATTTGAGGGGCAGACA
GAGAGGAGTGGGCTTTTAAAAGTACAGTTCTGAGACCCTTGGAGTGGAGCTGACGACACACTCCTCAGGG
TGTCAGTATTGCTTTTGTCCTATTTCTGGCAGAGGTATGAAGGTTATTGAAAACCGGGCCTTAAAAGATG
AAGAAAAGATGGAACTCCAGGAAATCCAACTCAAAGAAGCTAAGCACATTGCAGAAGAGGCAGATAGGAA
GTATGAAGAGGTAAGTGACCTTCTGTTAGTCCCTCTCGTATCGGCCTTTTACAAAGCATAATGCGGTAAA
TGGCTCCTTTGCTTCACTAAGCTATACTGGGATCTTTTCCTGTAGGTGGCTCGTAAGTTGGTGATCATTG
AAGGAGACTTGGAACGCACAGAGGAACGAGCTGAGCTGGCAGAGTCGTGAGTATCTAGCTCCCCAAATCT
TGTCATTACCAATTTATCCCTTCCCCTTTGGCTTGGGCACTAATATGAATAAATTAGCATTCCTTCAACC
TTGTTAAACAGGGCCTAGGGAATGGGAGTCATCTATAGTGCTTTTTTAGGCCCAAGAATGGTTTTAGGCT
TCCTTATTCTCCTGACTAGTGACAAGTGACAAAAGAGGCTTTCTTATTTTTTGTTGGTAGGGTTTACAAA
TTGAGAGCAAATAAGGACTAATACATGGTGAACAGTTAATTGGAAGATCCTACTGGGTCCTTCCACCTGG
GGTGGAAGACATCAAAGTGGCTATCCTGATTTAAGATCTAGTGTCTGGCTGGGTGTGGTGGCTCAAACCT
GTAGTCCCAGCTACTTAGTATGCCAAGGCAGGAAGGTTGCTTGAACCCAGGAGTTGGAGGCCAGCCTGGG
CGTCATAGGGAGACCCTGTCCCTAAAACAAATAAATTTAAAAAAAAAATCTGGTGTCTGTTTTGGCCACT
TTGCCAATCTGCTAGGGAACCTTAGGGCAAGGGTAGATATTAACTGCTCTCCCGTCGGTACCTTCTGGGC
CTTGATTAACAAAGTCAGTGCTGAATGCATATGTATTTGTGCTTCCCTCTTACATTCCCGTCTGCCAGAC
ATAACATATCATGACCATGGTGGTGTTCTCTTCCCATTTTTTGAATTTTTTTTTTTCGTTTTGCTGCTTG
TTTTCTTGCTGTGTTCCTTTTTGACCTTTCTCCCTCCCCACAACCTGGCTTGTGCCATCCCCGTGACTAT
CACTAACAGCCGTTGCCGAGAGATGGATGAGCAGATTAGACTGATGGACCAGAACCTGAAGTGTCTGAGT
GCTGCTGAAGAAAAGGTACTAACCATTAAGCCCACAGCAAGGTTATATATGTGGTGTGGTTTTGTTTTGT
TTTGTTTTTGTTTTGCAGCTGCCTCCCCTCCACTTGATTTTGTTGAGTGCTGTCTCATCTCTTCATGGAA
TGCTCCATTCTTTTCCACTTACTTGGCATCTTTCATCTCTTCTTTCCTAGTTGCTTCACTGCCTCTCCAC
AGGGTCTCCTTCCTTTGGGTGATTTGGGGGACTGTTTGGGTTGGGCATTTGTTCCCACCTCCACCCCAGG
TAGCAGTTATGACTTGTTCCTGGTAGTGGATTTCTCTCAAGTGCTTTATTAGTATTCAAACATTTTTGAG
CCCAGGAAGGTCTAGCTCCTGACACGTTCTATGGTAGAGGGAGGAGGGTTGATGCTTGCTCAGGTTACTT
GGGAACATCTCTTCCCCAGTATGCCTTCCAACTCTCTCTACATATAGGTTCAATTCACGTCTATGTGTCT
CTCTCCCTTTTTTTATTCTCTTCCTTTTCCCTTTTCCTCTCCTGTGGTATTTAAAATATTCACAGTAAGT
GTTCTGAGCTGGAGGAGGAGCTGAAGAATGTCACCAACAACCTCAAGTCTCTTGAGGCTCAGGCGGAGAA
GGTAGGGGCTGGTTTGTGAAAGTGACAGGCTTTGGGGCCTGGGCCCAGCCCTGCCCTTGATGAAAGACTG
GAGACACGCATTCCATATATAATCCAGTGAGGCGTGTCCTCTTCCTACCTCCTTACCATGTATTCAGGTT
CTGGGTTTAATGCTATTTTCTGTGCACTTTGATTGCTTTAATTAAAATAACTTCTTGGTCTCAGGTTACT
GGAGTCATGTGCTCCTTAGGCTGATACTGCTGTGCTGAAAAGCAATAATCAAGGGCCCTAACAGGCTTTC
TAGCAGACAGATGGGGGCAACAGACAGAATCAGCTCTGAGACCTAATGAGCAATCAAGACAGATGACTTC
ACTTTGCCATCATGTGGGTGTGTTTCAGTCATGTGACCTTGGGGTGCTACTACTCTTGCTCATCAAGCAG
GGGAAAGGAAAAACACCTCCTTGGGTTGATCTGAATAATATCAAAGCTTAGATCCATCTTAAAAAGTCAT
GGAGAATTTTAACCTGAGGATCTCATGGCTCTAAAGACATAGATTTCTGAATTTCAAGGACCTATTGTCC
AGAGCCAGGCCTTGCTCTTACTCTTGGACCATTCAGCACATTTAGTGCAAGAGGATATTCAATGTAAGTT
AAATGTAGGGAGGGGGGTATTGCCTGTGTGACTAATTTCTACTCTGAAATTTTTAATCTACTTATCTTAC
AGTACTCTCAAAAAGAAGATAAATATGAGGAAGAAATCAAGATTCTTACTGATAAACTCAAGGAGGTGAG
CTGAGGGGATTTATAGGGCAGAACCCAAAACATTTAGAGGTATATAAGGCATTAATATGGATTTAAACTG
GCTGGTTTGGGTTCTGAAAGGTCCAGAAGTAGAAGGTGGAAGAAATAGAGATTGTTCTTCTTTGTCTTCA
TCTCTGGTTCTGACTAATTTCATATTTCCCCCAGGCAGAGACCCGTGCTGAGTTTGCTGAGAGATCGGTA
GCCAAGCTGGAAAAGACAATTGATGACCTGGAAGGTATGGAACCTGGAGTAGGGATTGAAATAGAGAAAT
ATAGAGGAAGACTGCCCATTAACCATTAATCTCTTTTTCTGTACACCAAAGGAAGTTGGTAGTGACTTGG
CATGTATTAATATCATGTGCACTGTCCTATGCCACAGCTAGGCAAAGTGGGTCAAACTCTAGAATTGGTT
TTTTAAATTTAATCTTTTCCTTTAAGAAGACTTTACCAAATAGTCTGCAATTTGTACCCTTCCCTTTTGA
GGAGAATGATAATGGCTGCATTTACCTCCAATTTATTAGTCTTAAACCTTTAGTCTGTGGGTTTAAATGG
AAGCCATGATTTCAGAAGGGCAAGATGTATGTCATTTCATGGCTACTTTCCACAGTTCTTTTTGCCACTC
AAGTTGTCGGGTATTTATCCAATAAATGAAGTTGTGGAATAACCATATCTGAACATTGACCAATGTCAGG
AGCTTTCTAAATACTTTCTGTCCCTGCAAGTTGGTAACTGCCATCCACCCTGACAAGGGAACGTACTCTT
TGCTTTGAGAACTCTGATGTATGCTCCTTTGGTACACTCTCTTGGCAGAGTTGCAGGGGCAGCCTGTGGA
GCAGAGGGGCCACTAATAGGTTCACATTTTGTAGGCAGAGAAGTGTCTGAATATGGATATATACTCTCTC
CCTTTGCTATTTTTGTTCAATTAAGTGTTATCACTGCTAGGAATTGGGTACCTGGATGGGAGTGGTATCC
TCTTGAGTGCTTTTGGTAATAGGAATTTCTAGTTATGACTGTGCCCAGGTTCTCAGCCCTTGCTGCAGTC
GATTTGGAGTATCAAGAAAGGAAGTTGGTCATAGGAGAGAGTAGATCTGAAAATGTCCAGTCATGGTTGC
CAGAGTAGATATTTTCCTAACTGTGGTATTAGACCCACAGTCACACTTTCCTAATTTCTAATGATTCCCT
CTCTTCATCTGCTTCTGAAACTCTTCACTCTGTCCCCTCATTCCTCCCTGTGGTTCCTGTGCCTGTCCAG
ATGAGCTCTATGCCCAGAAACTGAAGTACAAGGCCATTAGCGAGGAGCTGGACCACGCCCTCAATGACAT
GACCTCTATGTAACTATCTGACAGTAGAGTGGGGCTGGGATCTTGGCTTGTGGGTGGATGGGGGTACAAG
CTATGCATAGTGGTGTGCTTTGGTTTTATCCTTTGGAAACTAGAATCTCCACCCTTATCTTTGAAACATT
GGTGCTGGTTATTTGTTTGGCAAAAGCTTTGGAGGCATGGCCGGGCGCGGTGGCTCACGCCTGTAATCCC
AGCACTTTGGGAGGCCGAGGCAGGTGGATCACGTGAGGTCAGGAGTTGGAGACCAGCCTGGCCAACATGG
TGAAACCCCGTCTCTACTAAAAATACAAAAAGTAGCCAGGCATGGTGGCAGTGCACCTGTAATCCTAGCT
ACTCAGGAGGCTGAGCCAGGAGAATCGCTTGAATCTGGGAGGCAGAGGTTACAGTGAGCCAAGATGTGCC
ACTGCATTTCAGCCTGGGCAACAGAGTGAGACCCCATCTCAAAAAAAAAAAAAAAGAAAAACATTAAAGG
CTGGGCGTAGTGGCTCACACCTGTGATCCCAGCACTTTGGGAGGCTGAGGTGGGCGGATGACTCGAGGCC
AGGAGTTCAAGACCAGCCTGGCCAACATGGCAAAACCCTGTCTCTACTAAAAAATATTTAAAAAATTAGT
TGGGCTTGGTGGCGCATGTCTATAATCCCAACTACTCGGGAGGCTAAGGCAGGAGAATCACTTGAAACCA
GGAGATGGAGGTTGCAGTGAGCCGAGATAATGCCACTGCACTCCAGTCTGGGTTGAAGAGCAAGACTGTC
TAAGAAAAAAAAGCTCTGGAGGCAATTAATCTTGGATCAGAAGGAGAACCCTGACTGACTTGTAATTTTT
ATATTTTGTATTCATAGTTTCTTCATTATACTGTGATTTTTCTATTTGCTTCTCAAATTTAGTCTTCTCA
GAAGGGATACTGCTAGAGGTAGAATCCATACAACTAAGGAAATAGGGCCCACAGAGCCAGTAACTTGGGC
CCTGACACATTAAGACAAAATTCAGGCCTCCTGGGTGTGTTTAATTGGTTCCCTGATATTAAAGTTCAGG
GAACTACCCAAGATGGGAAATACCAAATTCACCTAAGAATTGAGCTGAGTCCCAGAAGCAAGCCAAGTGA
TAAACAGCACCAAAAAGAGTTGTTGGGGCTTCATCTGTTTGCTGTGGATCCCTGATCCTTGATGCTAATC
TGCCTCTTTGTATCTTTCCCACTAACCCTGAAAAGAAGCCACATTTCTCAGGCTGAAGTGTCTGGCTCTC
TTTTATTATTCCTGCTGCCACCTCTTCCTTTTTTTCCTCTTCCTTTTTCCCAGTTTGCTATCTAGATTGA
TGCTAGTCCTTCTCACCTAGAGTATCCTTACTTTTTCATACAGATAATTATCACCGTTTCTGCTCTGTTC
TGGATCTGCCCCCTTTACTCCTCGGGGAACCCAAGGCCCCACTCTCGCTCTGGATTCCATTTGGGTCAGC
CTGGCTGGTCCCCAAGGCATTAGGATGGGGGAGCAAAAAGCAACTTATGTATTTTCTTCCACCCCCACCC
CAAATTAAAATGTTAAGCTGCTGGAAACCTCATGCCACCCTGCATTTGTGTCATTGACAAAGCTGTTGCT
GTCCCTAAGAAGGAGCCTTGGGGGTGTGATGTGGGGAAGAGCTATTGTAGGCTCCCCCTCCTCTGACTTA
TGTAATCAAAGCCACTTTTGTGTGTGTCTATTTTTTCTTGACATTTAAACTCAGCTGATCTGATTCTACC
AGAGTGATGGATTTAGTACAGGTTACTCAGGATAGTAATTTTAGTTATACTCCTCAAGCTGAACAAGATT
AAATTCCTTATTTCCAGGTTCTTAAATCATCCTGCCTGCAGTGTTTCCATTCTCTCTTCAGGTATTCCTC
CTTTGGTGTGGTGTCATTGAGAAGCCATTGAAGTGACTCTCAATACACATTCTGTACCCTTTTACCGGTG
GTTCAAATGGTGCATCCTCAGAACACCCAGTGAACCCAATACATTATTGCTAAGATTGACTAATTATGTC
AACTCCAGTCACAGAAAAATACACAATGGATAGAATTCTGGACGGTTTTTTTACTTTTTCTTCTTTAAAC
CTTTCTTACATATTTGAGACTTGCTACCATTTGCCTGCTAGTGTGTGACTAGTGGGATATAAGATAAGTG
ATAAATTATTATTGGGAAAACTAAAATGACCAATCATGCATATTTCAAATAATGTGCATATGAGGCTAAT
GATTTATTACATACATAAATTTCTGCTAGTAAAATTTTCCTTGGTTCATATTGTGGAATTAAATATCAAC
ATTTTAGAAATTCCCATTATAGGCCGGGCGTGGTAGCTCACGCCTATAATCCCAGCACTTTGGGAGGCTG
GGGCAGGTGGATCACCTGAGGTCATGAGTTCGAGACCAGCTCGGCCAACATGGTGAAACCCCGTCTCTAC
TAAAAATACAAAAATTAGCTGGGCATGGTGGGTGGGTGCCTGTAATCCCAGCTACTCGGGAGGCTGAGGC
AGGAGAATCGCTTGCACCTGGGAGGCGGAGGTTGCAGTGAGCGGAGATCGCGCCACTGCATTCCAGCCTG
GGCAACAGAGAGACTCCATCTCAAAAAAAAAAAAGGAATTCGCATTCCAATTTACACAGCAGAGATTTCT
TAATAGTATAGCTGTGAATTATACTAATCCAAGCACGTAAGTGTTGTTCACTTAGTACTTTAGTTTCCCA
GCAGGGTATGTATTTAAAATTTGCTTTTCTTTTAGCTGGGTGCAATGGCACTTGCCTGTACTGCCTGTAG
CTTCTTGGGAGGCTGAGGCAGGAGGATTGCTTGAGCCCAGGAGTTCTGCACTGTAGTGTGCTATGCAGGT
CAGATGTCCTCACTAAATTTGGCATCAGTATGGTGACCTCCTGGGAGTGGAGAACAACAAGGTTGCCTAA
GAAGGGGTGAACTGGCCCAGGTCGGAAATGGAGCAGGTCAAAATTCCTGTGCTGATCAGTAGTGGGATTG
CACCTATAAATAGCCACTGTAGTACTCCAGCCTGGGCAACACAGCAAGATTCCATCTGTTAAAAACACTT
TTGCTCTTCTTTTAAACAGATATAATCAGGTAGGGAAGTTTTCCTTAATCTAAAACTTTAAGTCATATCA
AATTCACGTTTCCTTTTGTCCATTCCATTTCTTACTGCCGCATAGCCTTTGAGATTAAGGTTCAGATTCA
TTTATTCAGCTGACACTTCCTGGTATCTTCTAGGTATAGGATATTAAGATGGAGACAGAAATAAAGATGG
AGGCACCATTCCTACTCTCAAGTTGCTTAGAATCTACTAGGAGAGAAAACATACATAAAGCTACAGATAC
TGTGTGATGAATGCCACAAATAATAGTGTTATGGGAATTGAGTAGAGGGGACGAGATTGATCCTAGTGGG
AATGAATTGGAAAAACTCATGGAAGAAGGCATGTAATGGGTACTTCATAAACATGTTAAAACTTTTTTCT
TTTTTTCTTTTTTTTTTTTTTTTTGAGACGGAGTTTGACTCTTGTCACCTAGGCTGGAGTTCAGTGGCCT
GATCTCAGCTCACTGCAACCTCTGCCTCCTGGGTTCAAGCAGTTCTCCCACCTCAGCCTCCCGAGTAGCT
GAGATCACAGGCGCCTGCCACCACACCCAGCTAATTTTTGTATTATTAGTAGAGGTGAGGTTTCACCATG
TTGGCCAGGCTGGTCTGGAACTCCTGACCTCAAGTGATCCACCCACCTCAGCCTCCCAAAGTGATGGGAT
TACAGGCTTGAGCCACTGCGCCCAGCCAACATGTTAAACTTGCATATTCATTTTTATTGGGTTCCAAAAG
TGGATTTTTGACAAGCAGAGTTGGGTTGTGGGATAGTGAATATTGAGCATTCCATTATTTATTTATTTTT
CTCTTTTTGAGATGGAGTCTCGCTCTGTCACCCAGGCTGGATGGAGTGCAGTGGTGAGATCATGGCTCAC
TGCAACCTCTGCTTCTTGAGTTCAAGCGATTCTCCTGTCTCAGCTTCCCGAGGAGCTGTGATTACAGATA
CCCACCACCATGCCCAGCTGATTTTTGTATTTTTAGTACGGACGGGGTTTCAGCATGTTGGCCAGGCTGG
TCGCGAACTCCTGACCTCAAGTGATCCACCCGCCTCTGCCTCCCAAAGTGTTGGGATTACAGGCGTGAGC
CACTGCACCTGGCCTGAGCATTCCATTTAAAGAGAAACAAAATAATAAAGGCATTGATAGAGATGAGAAG
GCCACAATAGCTCTAAAGGTAGATTTAGACAGAGTAGGGAACAGGTTGTGTCTTAAAATGAGGCTGAGGA
GTTATAATTTTACTTGGAGTTAATGGGGATGCCACTGGAGATTTTGAATAGGATATGTTGTGATCTGAAT
AACATATAAGAAGAATTAATTTGGTAACTAATTTGCAGATTAGCAAAGTGATTCACTAACATGGTGTTTC
TCACATTTCAGCCATTTATGTTACCATTTTTGATGGCTTTTTCCATATTCATTGCTGCCTACATTGTTTT
CTTCAGATCTACTCATTTCCAGATTTATTTTGTTCTTACTTGACACAATTCTATTTTGAAATCAAAGTTT
TGATGTGTTAGTGTTTTTTCCTAATCACATTAAAGTAACTATAGCAGTGAATACACCACACTTTGGAAAA
AATTGGGTTAGGTTAATTTAGTTTTCTAGGAGAGAATAATCAGGGTCTGAGCTAGAGTAGTGGCAGGAGA
GATAGCACTAAATAAAGACAGGCATTGTGAAAGGCCTGGCCACTTGATGTGAGGAGGAAAGACAAGGATG
ACTGGTTTTTAGAGTAGGAGAATTAGCCGGGTGCAGTGGCTCATGCCTGTAATCCCTGCACTTTAGGAGG
CTGAGGTGAGAGGATTTCTTGAATCCAGGAGTTCGAGACCAGCCTGGCAATAAAGTGAGAACCTGTCTCT
ACAAAATATAAAAACTTAGCCAAACATGGTGGTGCGTGCCTGCAGTCCTATCTACTTGGAGGGCCGAAGC
CAGAGGATCCTTTGAGCCCAGGAGTCTGAGGCTGCAGCAAGCTGTGATCACACTGCTGCACTCCATCCTG
GGTGGCAGAATGAGACCCCCCCCAAAAAAAGAGTAGGAAAGTGGTGGGGTTAAAGATGTAGGGAGATCAT
GTTGTCCTGGTTTGGGGAGCTGGGGGAAGGGAAAGAAGATACGGCTGACAATCTGGCAGGTATCTGGGTG
AAAACAAAATTCTAGGGCGTGAGGCAGAGAGGTTGACATAAGAGAGTTGGGAATCCATATGGGGTACAGG
TAGTTTATGCCATGGAGGTAAATTAGAACCATGAAGGACAATTGCAAATACAAAAAAAAGAGGCCAGGCA
CGGTGGCTCACGCCTGTAATCCCAGCACTTTGGGAGGCCGAGGTGGGCGGATCACGAGGTCAGGAGATCA
AGACCATCCTGGCTGACACGTTGAAACCCCGTCTCTACTAAAAATACAAAAAATTAGCTGGGCGTGGTTG
CGGGCACCTGTAGTCCCAACTACTTGGGAGGCTGAGGCAGGAGAATGGCGTGAACCCGGGAGGCGGAGCT
TGTAGTGAGCTGAGATTGTGCCACGGCATTCCAGCCTGGGTGACAGAGCAAGACTCTGTCTCAAAAAAAA
ATTAAACAATGAGGAAAACGTTATGTAAGAGGAGGAACCAAATGGAACTGATTGGAGGGAAAGAAGACGG
TTGAGTAGTTCTTTAAAAAGTTCCGGGTTGGATGCTTTCTAGTGGTTTTTCAACAGAGAGGCTAAGAAGG
AAGATAACTGAAAATGGGACATGCATTGATACTTTAGTAATTTGAGGGTACATTTTGAGTGTTAAGAAGT
GAAGAGGAGGAGTCATTTTCAGGGGCAAAAGAGTGTATTGGTAGGAAGATTATATAGGCAATATAATTTA
CTTTAGAGCAGGGCTTTCCAACAGAACTTTTGGTAATGATGTCTAGTACATTAGCCACATGTGGCAGTCA
AGCACTCAAAACATGGCTAGTGTGGCTGAGGGGCTAAAATTTAATTGTATTTCATTGTAGTTCATTTAAA
TTTGGCTACCTGTGGCTAGTGGCTATTATATTGGACAATGCATTTCTAAATATTCAGATCCTGGGCAGAA
GTTGGCTTTGACATGAAGAAAGACTTCTTAGAAAGGAAGGAAGTAGACCGGGCATGGTGGCTCATGCCTG
TAATCCCAACACTTTGGGAAGCCGAGGCAGGTGGATCACAAGGTCAAGAGATCAAGACCATCCTGGCCAA
CGTGGTGAAAACCTGTCTCTACTAAAAATACAAAAATTAGCTGGACATGGTGGCACATGCCTGTAGTCCC
AACTACTCGAGAGGCTGAGGCAGGAGAGAAAAAAAAGAAGGAAATAAGAGGGAGATTGGGAGAAAAGTAA
GGTGTTATAGCTCTGATCTGTGGTATCTCCTGTTTTCCTTGCGGTATGACTAGTGTGAGCCAAGGATAGA
GACCAACAAACTGGGGTACCAAAGTGGACAATGAAGTACTATGTAATTAGTGCTAATGCTAAATTCATTC
CTTTGTTTAAGGCTTAATATCCTTGCAGAAGCCATCCTGTGTTACTTGAGCCTGGACTAGTATTGGGGTG
AAGTCAAGCATCAGAGTAAAACATTTGTCCCCTTAATGTTACCCCCTCTTAATTTCTAAGAACCACAGGC
CCATTTTGCCCTTCCTTGTTACCATCATTGAGATAAGGAATAAGATGTAGTAACCCCAAGTTATCTGATA
GGTACAACTGACCTATTCTGTTTGGCAGCTTTATCTTAGACCTATTCTGTTTGGCAGCTTTATCTTAGCC
CCAACCAAATCTCTTCCTTTCACATGGTGTTGGTTGAATACTATCCAACTCCATGGGGTTGGTTTGTGTT
TTTTTTTTTTTTTTTTTTTGAGACGGAGTCTTGCTCTGTTGCCCAGGCTGGAGTGCGGTGGTGTGATCTT
GGCTCACTGCAAGCTCCGCCTCCTGGGTTTAAGTGATCCTCCTGCCTCAGCCTCCTGAATAGCTGGGATT
ACAGGCACGTGCAACCAAGCCCGGCTAATTTTTGTGTTTTTAGTAGAGACTAGGTTTCAACATGTTAGGC
TGATCTCAAACTCCTGACCTCGTGATCTGCCCGCCTCGGCCTTCCAAAGTGCTGGGGTTACAGACTTGAG
CCACTGCACCTGGCCATGGGGTTGTTTCTTAATTAGATATAGCTGAAAAGAACGCTAGACCAAATAGGTT
CTCTGCCTTGCCTTTTCGTTTGTTTTGTTTTAGCTATTATCAGGGAACCAAAAACTTTAAGGAGCTAGTA
CTGGTCTTAATTTTTAATAACTAGAGATAGCAGAGTTAGAAACTAAGTTCAAAGTGAGAGAACAGCTGCA
TTTGTCTTTCTGACCTCATGCAATTCCTAGGAAACTCTGTGTTCTGTGATTTAGTCAGGCAATAAAATGC
TCTCACTCCTTCTCTGTCTCTTTATTTCTTCCCTAAATGGAAAAGAATTGACCAGGCTGCTTTGAGGGAT
AAAGATCCTGATAACGCGGCTGGGTGCGGTGGCTCATGCCTGTAATCCCAGCACTTTGGGAGGCGGGCAG
ATCACCTGAGGTCAGGAGTTCAAGACCAGCCTGGCCAATATGGTGAAACCCCATCTCTACTAAAAAAAAA
AAATGCAAAAATTACCTGGGCGTGGTGGTGGGTGCCTGTAATCCCAGCTACTCAGGAGGCTGAGGCGAGA
GAATCGCTTGAACCTGGGAGGTGGAGGTTGCAGTGAACCAAAATCGTACCATTGCACTCCAACCTGGGCA
ACAAGAGTGAAACTCAAAAAAAAAAAAAAAAAAAAAGGATCCTGATAACATCCTTCTTTCCCAAGTGAGA
ATCACACAAACTAACTTGGTTATAGGTATTATCTAAATTCAGGCTGTTCACGAGATCTATATGTAATGTA
ACATCACAGGAAATTTGAGAGATTAGTTCACAAATTTCAACACCTGTTATTTTGACAAGGACGCCCCAGT
GAATAAGACAGGTCCAATCTCAGGCCTCATGAAGCTTATATACCATTAGTATAGTTTTGAGTATTCCTTA
TTCAAAATGCTTGGGAGCAGAAGTGTTTTGGATTTTTGCTTTTTTTTTTTTTTTTTTTTTTTAATATTTG
CATTATACTTTCCAGTTGAGCATCCCAAATTCGAAAATCAGAAATTTATACCAGGCATGGTGATGCACAC
CTATAGTTCCTGCTACTCAGGAGGCTGAAGCAAGAGGTTTGCTTGAGCCCTAGGAGTTCAGGGCTGTTGT
GCACTATGATTAGGCCTATGAATAGTCACCACACTGTAGCCTGGACAACACAGTAAGACCTTGTCTCTAA
TACACACACACACAAACACACACATCTGTATGTGTATGTAAAATCCAAAATCCTCCAATGAGCATTTTCT
TTGAGCATCATGTTGGCATTCAAAAAGTTTCAGATTTTGGAGCATTTTGGATTTTGGACTTTCAGATTTG
GGTTCTCAGCCTGTATAGTGTTTGTCAATGGTGAACGTTTGTCATTTAGAGTAGGACAGTTCTGTTGATG
TACAGAACAATCCTGTGTATTGCTGTCCCCTGCCCAACAGATACCTGTATGGGCCTTCTAGTCACTGTGA
TATCAAAACCTCCCATACATACTCAAAATCTCCCCTTGGAGAGAAGAGTGCACAATACCACCCCTATTGA
GAACCATTGGTTTAATGGGGAGTGCAAGTTAAAGGTTCACCCTGATTCTACTGTTTAATACAGCTATTAT
TTTTAGTCATTCCCTCCCAGGCTTTTTACACATATATGCTTTCTGTTTTGATAGTGTACATAAAGTGGCA
AGTATTACTGGGACCATACTGGTCTCTAGCTTCCCAGTCAATCTGGGGAGAGCCTCAGCCCACAAGGCAG
GGTACCTGAGCAATGCTCACATATGCAGATGATTTTAGGACAGAAAAGTATCTTTTTCATATTGAGATCC
TGAGACCATTTTGAAACGGGCTGTAAAATATAGGGAGAAACTTATTTGTGAATTTCAGAGGTTGATACTA
GGAGTATACCAGGAAGAGTTTGGTTTTCCTGTCCCCAAAGTTGTATTATCCCTGTGCTAGTCAGATGTAT
TTGAATCTTCCATATCCCACTATCTCTTTAAAGTGTGCTTCAAATGGAAAATCCAGATAAACCTATTAGA
TTTGAGCACTTAGCTGCTTGTATCAGGAATGCTTGCTAAATTGTCCTTTAGCCTGATTTGTAAAAAAAAA
AAAAAAAAAAAAGGAAATAACTTACTTAGTAGCTTAAATAAATATGGGATTTGTTTTTTAATCACATACA
TCTAGCCATGAGCAACTGTGGCCTGTTGTAGCTGGGCTTTCCATGATTCTCTTGGTCTTTTCTCTTAAGA
TTGAAATATGCAACTCTGGTCATCACATCCATGTTGAAGGCAAGAGGGAAAGCTAAAGTTTTCCCAGAAG
CCCCCAGCAGACTATCTTTCTACATCTCATTGGTTAAAACTGGATCATATGATCACCCCTAGCTGCAAGG
GAGGCTGAAAAACAACAGGATAGTCTTGAGTAGCTTATACCAGTAATTTGCCTTCCCTGGGACTAGGCAC
ACTATTTGTTGACTCTTAACAGGTTTGGGGTTCTTTTAAAGAAGAAGAAGAAATTGAATATTTATTGGCT
AGGCCACTGAAATGGGTCTATCACACAATATCTGTAACACAAAGCATTAGAAAGTATAGGAGTGAATGAG
TGTGGGGATATTTCTCCCAGTATACTGTTTATAATGCTGTCTTGAATGCTTTCACAGAAAAATGTTCAGC
TGTTCACTTCCATTCCTTTCAAACCCTGATTATTGTGAACACATTTCTTCGAAGCCAACTGTATTCCTTC
CTTTGCCATTTCCCTTGTTTCAGTTTCTCAACCCAGGGAATAAATATTGTGAAGGAAGCATTTTTTCTCC
CTCAAAATGTTGTCCTACCAACTTGTCAGTCTGAATTTCCTCTGTTGGTCTTTCCACCAACTGACTGACT
CTTATTGCCCTCTATCTGAATCCTCCTTGCATCCACCCTGCCTTACAGATGCATGTTATATATACCAGCT
AGTTGAAAGACTCATAATTTACTTCTGGTACTCTACTAACTTTCCCTTTTTCTCTTTCTCCTCCTCTTTT
TCCTCTCCTTCTCTTGTGTTCCCCTCCTCTCCGTTGCTGCTGCAGAGCGTCTCTACAGCCAACTTGAGCG
AAACCGCCTGCTTTCTAATGAGCTGAAGCTAACGCTGCATGATCTGTGTGACTGATGGGCAGGGCTCAAT
GATGCCCATTAAACTGAGCTTACTGCTCACACCACTGACCTGGACCCCAACAAAAAGCTGATTGTCTTTT
TAAAAGTTATTATTTTGCCCTGAGCAAATTGCATTTTAATTGGGGCAGTTAGAATGTTGATTTCCTAACA
GCATTGTGAAGTTGACCATTGTGAAGTTTCTGTCCCTTTAGAAGAGATTATGGGTGAAGAAGGGAGGGGC
CTGAGAGATTATAGTGAGAAAACTTGCGAGAATTTTGTTTTCCACCCTTATTTGCTGCTCTTTCACTTGG
GCACTGACTGTAGGATATGTTCCCTTGCATGGATGTTTTTAACAATAAAAGGACTGACTTGACAAGTTGT
TGTAACTGCTTCATCGGCAGGCCCAGGAATGGGTCCTTCTGACTGGGTGGAAAAAAGGGAAGTGAAAGAA
AAGTTGTGGGATATGAATATGGGTCTGTGTTGCCATCACCTTCTCTGAGTTGAAGATTTGAGTATTTTCC
TCACCTCTTTAGAGCAGTCAGAGTGGTTTGCTTGCTAGACAGATTAGATTCTCCTTAATGTTCAGCTGCT
GATTTTCTTTCTGACTTTTGCGTCCTTTTTCTGGTTTTATGTTAATTTCAAGTAACTGTCACAAGCTAGT
TCTGTTCAATAGCTCTGCAGCAATCTCAAGGTTTGCTTACAACTACTTGTTTCAGTAGTATTCTTGGCTT
TGTTTTCTTTAGAGATTATTTGACTTAACTGTGAGCGCCCTTTTATTTATCCCATCAGTTATTACTTTGG
CCTCTACTTTTTCGAAAAAACATGTAGTGCATGAGGATCTTCCTGTGCTCTTTATAATCTGAGATTCTGA
TGTTTCTATTGTTTGCAATGTTCAAACTCCGGTGAGCCATTTCAAGAGGGTATTGTTATGTGGGCAAAAC
CTAGAAAAGTGGATGGCTGATGGTTAAGGCTTGCTCTTTCATTGACTGAAAGCTGAAAGTGTTGGTTGGG
TGTGGGAGGGAGAGGAAATGGCTGATAAGGGCCCTAACTCCCTCACCCAGGAAGTGCAGCAACACCTACA
ACTTCAGTAGGCAAGCCAAAGGCCCTACAAAACTGGGTGATGTAATAGCTCACTTCTGTGGCTGAGAAGG
CAGCTGCTTTATCAGTCTGCAGCTTCTCTGCAACAGGAGCAAGTCTCAAAGAGCGGGTAGACCTTGAAAT
TTACTTCTAGTTCTTGTAACTTCTCTCCTTTACCCCCATTAGATAAACTGAAATGCACCAAAGAGGAGCA
CCTCTGTACACAAAGGATGCTGGACCAGACCCTGCTTGACCTGAATGAGATGTAGAACGCCCCAGTCCCA
CCCTGCTGCTGCTCCTCCCTCTGACCCAGACTCCGCCTGAGGCCAGCCTGCGGGAAGCTGACCTTTAACT
GAGGGCTGATCTTTAACTGGAAGGCTGCTTTCTCCTTTCACCACCCCCTCCTTCCCTGTGTCTTTTTCGC
CAAACTGTCTCTGCCTCTTCCCGGAGAATCCAGCTGGGCTAGAGGCTGAGCACCTTTGGAAACAACATTT
AAGGGAATGTGAGCACAATGCATAATGTCTTTAAAAAGCATGTTGTGATGTACACATTTTGTAATTACCT
TTTTTGTTGTTTTGTAGCAACCATTTGTAAAACATTCCAAATAATTCCACAGTCCTGAAGCAGCAATCGA
ATCCCTTTCTCACTTTTGGAAGGTGACTTTTCACCTTAATGCATATTCCCCTCTCCATAGAGGAGAGGAA
AAGGTGTAGGCCTGCCTTACCGAGAGCCAAACAGAGCCCAGGGAGACTCCGCTGTGGGAAACCTCATTGT
TCTGTACAAAGTACTAGCTAAACCAGAAAGGTGATTCCAGGAGGAGTTAGCCAAACAACAACAAAAACAA
AAAATGTGCTGTTCAAGTTTTCAGCTTTAAGATATCTTTGGATAATGTTATTTCTATTTTTTATTTTTTT
CATTAGAAGTTACCAAATTAAGATGGTAAGACCTCTGAGACCAAAATTTTGTCCCATCTCTACCCCCTCA
CAACTGCTTACAGAATGGATCATGTCCCCCTTATGTTGAGGTGACCACTTAATTGCTTTCCTGCCTCCTT
GAAAGAAAGAAAGAAAGAAGACTGTGTTTTTGCCACTGATTTAGCCATGTGAAACTCATCTCATTACCCT
TTTCTGGGTTTGAAGCTGCTGTCTCTAGAAGTGCCATCTCAATTGTGCTTTGTATCAGTCAGTGCTGGAG
AAATCTTGAATAGCTTATGTACAAAACTTTTTAAATTTTATATTATTTTGAAACTTTGCTTTGGGTTTGT
GGCACCCTGGCCACCCCATCTGGCTGTGACAGCCTCTGCAGTCCGTGGGCTGGCAGTTTGTTGATCTTTT
AAGTTTCCTTCCCTACCCAGTCCCCATTTTCTGGTAAGGTTTCTAGGAGGTCTGTTAGGTGTACATCCTG
CAGCTTATTGGCTTAAAATGTACTCTCCTTTTATGTGGTCTCTTTGGGGCCGATTGGGAGAAAGAGAAAT
CAATAGTGCAACTGTTTTGATACTGAATATTGACAAGTGTCTTTTTGAAATAAAGAACCAGTCCCTCCAA
CCCTCAGACCTATTTGACTTTTATTTATTAAAACTAAATGTGCTTTCTCCACAGAAGCTATGAGGTTTGG
GTTAAAAATAGCATCTTTGTGGGTGGTAGCAACAGGATTTATTCTTTATTATTATTATTTTTGAGATGAA
GTTTCATTCTTGTTGCCTGGGCTGGAGCGTAATGGCTCGATCTCGGCTCACTGCAACCTCCGCCTCCTGG
TTCAAGAGATTCTCCTGCCTCAGCCTCCCGAGTAGCTGGGATTACAGGCACCTGCCACCATGCCCGGGTA
ATTTTTTATATTTTAAGTAGAGACAGGGCTTCACCATGTTGGCCAGGCTGGTCTCGAACTCCTGACCTTC
AGGTGATCCACCTGCCTCAGCTTCCCAAAATGCTGGGATTACGGGCGTGAGCCACCGCACCCAGCTGGAG
CAACAGGATTTAATATAGAGCAAATGTTTAGTTTTATCATCTGTAAAATGGAGATAAGTATTGTCAGAGT
AAACATGAAGATTAGAAAGAACACTTAATGTGCTGGGCCTTTTATAGGTTAACACTGACATCTCAGGCTG
AACTATATACATTTTCCTTCACAACCATATCAATCCTTATAAACTATGGATTTATGCTCCTTAAAACAAT
ATATAATGCTGATCACTACTATAAATGCGTGGTTTTAACCAACTGTACTGAAACAGCTTTGAGTTTATAT
TCTGTTTGGATATTTGGAGAAAACAACAAGTGCTCTCAAGAGTATTTGCTTAGAGGCCGGCTGTGTGAGT
GGATAACTTTGAAAGCTGCTTTTGAGACGCCAGTGTCTGGCATTTCCTGCATTCTGGCCTGGAGGCCGGA
CGTGAATCTGACTTCTAGTAAAAATACACGGTTCCCTTGACAAAGTCGAGCTGTTTATCCCAGAGACTGC
ACAATTTTCCGTTGATAGGCATGGACCAATGCTAACTGGAAATCATTGCAAAAAGTTTTTTTGTCGGGCG
GAGGGTGTGGTGTTAAGATAAACAGTGTGCAACAGAAGAAATTAAAACTGGAAGAAATTAAAGGGTTTTT
TTTAGACTTT

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 1%, 5%, 10%, 15%, 20%, etc. change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with exposure to alkylbenzenesulfonates (e.g., linear alkylbenzenesulfonate).

As used herein, “obtaining” as in “obtaining an agent” includes synthesizing, purchasing, or otherwise acquiring the agent.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 91%, 92%, 93%, 94%, 95%, 96%, 97%, 98%, or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a bar graph illustrating cytotoxic exemplary effects of LAS on Caco-2 cells as assessed by an MTT assay;

FIG. 2 is a bar graph illustrating that the exemplary cytotoxic effects of LAS on Caco-2 cells is not due to apoptosis;

FIG. 3 is a 2-Dimensional gel illustrating exemplary LAS-induced changes in protein expression in Caco-2 cells;

FIG. 4 is a protein spot matrix illustrating side by side comparisons of LAS-induced changes in protein expression at increasing concentrations of LAS;

FIG. 5 is a table illustrating molecular characteristics of the exemplary proteins identified in FIG. 3;

FIG. 6 is a bar graph illustrating exemplary changes in THIO mRNA levels relative to a control at 3, 6, 12, and 24 hours after LAS exposure;

FIGS. 7A-7D are bar graphs illustrating changes in ROS levels relative to a control at 1, 3, 6, and 12 hours after LAS exposure, respectively;

FIG. 8 is a bar graph illustrating exemplary changes in TPM3 mRNA levels relative to a control at 3, 6, 12, and 24 hours after LAS exposure;

FIG. 9 is a line graph illustrating exemplary changes in the relative electrical resistance of Caco-2 cells exposed to 5, 10, 30, and 60 ppm LAS over time, as assessed by a TEER assay;

FIG. 10 is a bar graph illustrating exemplary changes in CALR mRNA levels relative to a control at 3, 6, 12, and 24 hours after LAS exposure;

FIG. 11 is a bar graph illustrating exemplary changes in intracellular calcium levels relative to a control at 3, 6, 12, and 24 hours after LAS exposure; and

FIG. 12 is a bar graph illustrating exemplary changes in HSP7C mRNA levels relative to a control at 3, 6, 12, and 24 hours after LAS exposure.

### DETAILED DESCRIPTION OF THE INVENTION

The invention features compositions and methods for cytotoxic effect measurement and risk assessment of linear alkylbenzenesulfonate (LAS) in the environment, and more particularly, an aqueous environment. The present invention is based, at least in part, on the discovery of four LAS biomarkers that allow an accurate risk assessment of LAS contamination in a sample.

### Diagnostics

The present invention features diagnostic assays for the detection of LAS in an environmental sample (e.g., a water sample). In one embodiment, levels of any one or more of the following LAS biomarkers CALR, HSP7C, THIO, and TPM3 are measured in a sample and used to assess the risk of LAS contamination in the sample. In other embodiments, levels of CALR, HSP7C, and/or THIO, are characterized in a sample. In some embodiments, levels of CALR, HSP7C, and/or TPM3 are characterized in a sample. In other embodiments, levels of CALR are characterized, alone, or in combination with HSP7C and/or THIO and/or TPM3.

Standard methods may be used to measure levels of a marker in any environmental sample, which may include water samples, sewage samples, waste water treatment plant samples, soil samples, biological samples, etc. Methods for measuring levels of polypeptides include immunoassay, ELISA, western blotting and radioimmunoassay. Elevated levels of HSP7C alone or in combination with one or more additional LAS biomarkers are considered a positive indicator of LAS risk. The increase in HSP7C, CALR and/or TPM3 levels may be by at least about 5%, 10%, 15%, 20%, 25%, 30%, 40%, 50%, 60%, 70%, 80%, 90%, and 95% or more. In one embodiment, a decrease in the level of THIO may be associated with a positive indication of LAS risk.

Any suitable method can be used to detect one or more of the markers described herein. Successful practice of the invention can be achieved with one or a combination of methods that can detect and, preferably, quantify the LAS biomarkers. These methods include, without limitation, hybridization-based methods, including those employed in biochip arrays, mass spectrometry (e.g., laser desorption/ionization mass spectrometry), fluorescence (e.g. sandwich immunoassay), surface plasmon resonance, ellipsometry and atomic force microscopy. Expression levels of markers (e.g., polynucleotides or polypeptides) are compared by procedures well known in the art, such as RT-PCR, Northern blotting, Western blotting, flow cytometry, immunocytochemistry, binding to magnetic and/or antibody-coated beads, in situ hybridization, fluorescence in situ hybridization (FISH), flow chamber adhesion assay, ELISA, microarray analysis, or colorimetric assays. Methods may further include, one or more of electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APC)-MS), APCI-MS/MS, APCI-(MS)n, atmospheric pressure photoionization mass spectrometry (APPI-MS), APPI-MS/MS, and APPI-(MS)n, quadrupole mass spectrometry, fourier transform mass spectrometry (FTMS), and ion trap mass spectrometry, where n is an integer greater than zero.

Detection methods may include use of a biochip array. Biochip arrays useful in the invention include protein and polynucleotide arrays. One or more markers are captured on the biochip array and subjected to analysis to detect the level of the markers in a sample. Markers may be captured with capture reagents immobilized to a solid support, such as a biochip, a multiwell microtiter plate, a resin, or a nitrocellulose membrane that is subsequently probed for the presence or level of a marker. Capture can be on a chromatographic surface or a biospecific surface. For example, a sample containing the markers, such as serum, may be used to contact the active surface of a biochip for a sufficient time to allow binding. Unbound molecules are washed from the surface using a suitable eluant, such as phosphate buffered saline. In general, the more stringent the eluant, the more tightly the proteins must be bound to be retained after the wash.

Upon capture on a biochip, analytes can be detected by a variety of detection methods selected from, for example, a gas phase ion spectrometry method, an optical method, an electrochemical method, atomic force microscopy and a radio frequency method. In one embodiment, mass spectrometry, and in particular, SELDI, is used. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

Mass spectrometry (MS) is a well-known tool for analyzing chemical compounds. Thus, in one embodiment, the methods of the present invention comprise performing quantitative MS to measure the serum peptide marker. The method may be performed in an automated (Villanueva, et al., Nature Protocols (2006) 1(2):880-891) or semi-automated format. This can be accomplished, for example with MS operably linked to a liquid chromatography device (LC-MS/MS or LC-MS) or gas chromatography device (GC-MS or GC-MS/MS). Methods for performing MS are known in the field and have been disclosed, for example, in US Patent Application Publication Nos. 20050023454; 20050035286; U.S. Pat. No. 5,800,979 and references disclosed therein.

The protein fragments, whether they are peptides derived from the main chain of the protein or are residues of a side-chain, are collected on the collection layer. They may then be analyzed by a spectroscopic method based on matrix-assisted laser desorption/ionization (MALDI) or electrospray ionization (ESI). The preferred procedure is MALDI with time of flight (TOF) analysis, known as MALDI-TOF MS. This involves forming a matrix on the membrane, e.g. as described in the literature, with an agent which absorbs the incident light strongly at the particular wavelength employed. The sample is excited by UV, or IR laser light into the vapour phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are commercially available from PerSeptive Biosystems, Inc. (Frazingham, Mass., USA) and are described in the literature, e.g. M. Kussmann and P. Roepstorff, cited above.

Magnetic-based serum processing can be combined with traditional MALDI-TOF. Through this approach, improved peptide capture is achieved prior to matrix mixture and deposition of the sample on MALDI target plates. Accordingly, methods of peptide capture are enhanced through the use of derivatized magnetic bead based sample processing.

MALDI-TOF MS allows scanning of the fragments of many proteins at once. Thus, many proteins can be run simultaneously on a polyacrylamide gel, subjected to a method of the invention to produce an array of spots on the collecting membrane, and the array may be analyzed. Subsequently, automated output of the results is provided by using the ExPASy server, as at present used for MIDI-TOF MS and to generate the data in a form suitable for computers.

Other techniques for improving the mass accuracy and sensitivity of the MALDI-TOF MS can be used to analyze the fragments of protein obtained on the collection membrane. These include the use of delayed ion extraction, energy reflectors and ion-trap modules. In addition, post source decay and MS-MS analysis are useful to provide further structural analysis. With ESI, the sample is in the liquid phase and the analysis can be by ion-trap, TOF, single quadrupole or multi-quadrupole mass spectrometers. The use of such devices (other than a single quadrupole) allows MS-MS or MSⁿ analysis to be performed. Tandem mass spectrometry allows multiple reactions to be monitored at the same time.

Capillary infusion may be employed to introduce the marker to a desired MS implementation, for instance, because it can efficiently introduce small quantities of a sample into a mass spectrometer without destroying the vacuum. Capillary columns are routinely used to interface the ionization source of a MS with other separation techniques including gas chromatography (GC) and liquid chromatography (LC). GC and LC can serve to separate a solution into its different components prior to mass analysis. Such techniques are readily combined with MS, for instance. One variation of the technique is that high performance liquid chromatography (HPLC) can now be directly coupled to mass spectrometer for integrated sample separation/and mass spectrometer analysis.

Quadrupole mass analyzers may also be employed as needed to practice the invention. Fourier-transform ion cyclotron resonance (FTMS) can also be used for some invention embodiments. It offers high resolution and the ability of tandem MS experiments. FTMS is based on the principle of a charged particle orbiting in the presence of a magnetic field. Coupled to ESI and MALDI, FTMS offers high accuracy with errors as low as 0.001%.

In one embodiment, the LAS biomarker qualification methods of the invention may further comprise identifying significant peaks from combined spectra. The methods may also further comprise searching for outlier spectra. In another embodiment, the method of the invention further comprises determining distant dependent K-nearest neighbors.

In an additional embodiment of the methods of the present invention, multiple markers are measured. The use of multiple markers increases the predictive value of the test and provides greater utility in LAS risk assessment.

Expression levels of particular nucleic acids or polypeptides are correlated with LAS risk. Antibodies that bind a polypeptide described herein, oligonucleotides or longer fragments derived from a nucleic acid sequence described herein (e.g., an CALR, HSP7C, THIO, TPM3, or any other method known in the art may be used to monitor expression of a polynucleotide or polypeptide of interest). Detection of an alteration relative to a normal, reference sample can be used as an indicator of LAS risk. In particular embodiments, specific alterations (described further below) in the expression of CALR, HSP7C, THIO, and/or TPM3 polypeptides are indicative LAS risk. In other embodiments, a 2, 3, 4, 5, or 6-fold change in the level of a marker of the invention is indicative of LAS risk. In yet another embodiment, an expression profile that characterizes alterations in the expression two or more markers is correlated with LAS risk.

The polymerase chain reaction (PCR) is a technique for amplifying or synthesizing large quantities of a target DNA segment. PCR is achieved by separating the DNA into its two complementary strands, binding a primer to each single strand at the end of the given DNA segment where synthesis starts, and adding a DNA polymerase to synthesize the complementary strand on each single strand having a primer bound thereto. The process is repeated until a sufficient number of copies of the selected DNA segment have been synthesized.

During a typical PCR reaction, double stranded DNA is separated into single strands by raising the temperature of the DNA containing sample to a denaturing temperature where the two DNA strands separate (i.e. the “melting temperature of the DNA”) and then the sample is cooled to a lower temperature that allows the specific primers to attach (anneal), and replication to occur (extend). In illustrated embodiments, a thermostable polymerase is utilized in the polymerase chain reaction, such as Taq DNA Polymerase and derivatives thereof, including the Stoffel fragment of Taq DNA polymerase and KlenTaq1 polymerase (a 5′-exonuclease deficient variant of Taq polymerase—see U.S. Pat. No. 5,436,149); Pfu polymerase; Tth polymerase; and Vent polymerase.

The diagnostic methods described herein can be used individually or in combination with any other diagnostic method described herein for a more accurate LAS risk assessment.

As indicated above, the invention provides methods for assessing the risk of LAS contamination, as specified herein. These markers can be used alone, in combination with other markers in any set, or with entirely different markers in aiding in LAS risk assessment. The markers are differentially present in cell populations that have been exposed to LAS relative to control populations that have not. Therefore, detection of one or more of these markers in a sample would provide useful information regarding the probability of a LAS risk in a given sample.

The detection of the LAS biomarker is then correlated with a probable LAS risk. In some embodiments, the detection of the mere presence of a LAS biomarker (e.g., THIO), without quantifying the amount thereof, may be useful and may be correlated with a probable risk of LAS contamination. The measurement of markers may also involve quantifying the markers to correlate the detection of markers with a probable assessment of LAS risk.

The correlation may take into account the amount of the marker or markers in the sample compared to a control amount of the marker or markers (e.g., a known amount of LAS). A control can be, e.g., the average or median amount of LAS present in sample. The control amount is measured under the same or substantially similar experimental conditions as in measuring the test amount. As a result, the control can be employed as a reference standard.

Accordingly, a marker profile may be obtained from a cell population exposed, as described in greater detail below, to a sample and compared to a reference marker profile obtained from a reference population, so that it is possible to classify the sample as posing a LAS risk, or not.

### Real-Time PCR

Thermocycling may be carried out using standard techniques known to those skilled in the art, including the use of rapid cycling PCR. Rapid cycling techniques are made possible by the use of high surface area-to-volume sample containers such as capillary tubes. The use of high surface area-to-volume sample containers allows for a rapid temperature response and temperature homogeneity throughout the biological sample. Improved temperature homogeneity also increases the precision of any analytical technique used to monitor PCR during amplification.

In accordance with an illustrated embodiment of the present invention, amplification of an LAS biomarker nucleic acid sequence (e.g., mRNA, cDNA, etc.) may be conducted by thermal cycling the nucleic acid sequence in the presence of a thermostable DNA polymerase using the device and techniques described in U.S. Pat. No. 5,455,175, the disclosure of which is expressly incorporated herein. In accordance with the present invention, PCR amplification of one or more targeted LAS biomarker nucleic acid sequences may be conducted while the reaction is monitored by fluorescence.

The first use of fluorescence monitoring at each cycle for quantitative PCR was developed by Higuchi et al., “Simultaneous Amplification and Detection of Specific DNA Sequences,” Bio. Technology, 10:413-417, 1992, and used ethidium bromide as the fluorescent entity. Fluorescence was acquired once per cycle for a relative measure of product concentration. The cycle where observable fluorescence first appeared above the background fluorescence (the threshold) correlated with the starting copy number, thus allowing the construction of a standard curve. Probe-based fluorescence detection systems dependent on the 5′-exonuclease activity of the polymerase have improved the real-time kinetic method by adding sequence specific detection.

The amplified target may be detected using a TaqMan fluorescent dye to quantitatively measure fluorescence. The TaqMan probe has a unique fluorescently quenched dye and specifically hybridizes to a PCR template sequence, as described by Livak et al., “Allelic discrimination using fluorogenic probes and the 5′ nuclease assay,” Genet Anal. 1999 February; 14(5-6):143-9.), which is incorporated by reference in its entirety. During the PCR extension phase, the hybridized probe is digested by the exonuclease activity of the Taq polymerase, resulting in release of the fluorescent dye specific for that probe.

The amplified target may also be detected using a Pleiades fluorescent probe detection assay to quantitatively measure fluorescence. The Pleiades probe specifically hybridizes to a target DNA sequence and has a fluorescent dye at the 5′ terminus which is quenched by the interactions of a 3′ quencher and a 5′ minor groove binder (MGB), when the probe is not hybridized to the target DNA sequence, as described by Lukhtanov et al., “Novel DNA probes with low background and high hybridization-triggered fluorescence,” Nucl. Acids. Res. 2007 January; 35(5):e30), which is incorporated by reference in its entirety. By the end of PCR, the fluorescent emissions from the released dyes reflect the molar ratio of the sample. Methods for assaying such emissions are known in the art, and described, for example, by Fabienne Hermitte, “Mylopreliferative Biomarkers”, Molecular Diagnostic World Congress, 2007.

Alternatively, PCR amplification of one or more targeted regions of a DNA sample can be conducted in the presence of fluorescently labeled hybridization probes, wherein the probes are synthesized to hybridize to a specific locus present in a target amplified region of the DNA. In an illustrated embodiment, the hybridization probe system comprises two oligonucleotide probes that hybridize to adjacent regions of a DNA sequence wherein each oligonucleotide probe is labeled with a respective member of a fluorescent energy transfer pair. In this embodiment, the presence of the target nucleic acid sequence in a biological sample is detected by measuring fluorescent energy transfer between the two labeled oligonucleotides.

These instrumentation and fluorescent monitoring techniques have made kinetic PCR significantly easier than traditional competitive PCR. More particularly, real-time PCR has greatly improved the ease, accuracy, and precision of quantitative PCR by allowing observation of the PCR product concentration at every cycle. In illustrated embodiments of the present invention, PCR reactions are conducted using the LIGHTCYCLER® (Roche Diagnostics), a real-time PCR instrument that combines a rapid thermal cycler with a fluorimeter. Through the use of this device, the PCR product is detected with fluorescence, and no additional sample processing, membrane arrays, gels, capillaries, or analytical tools are necessary. Other PCR instrumentation, as known in the art, may be used in the practice of the present invention. LAS biomarker probes and/or primers may be chosen by any of a variety of techniques known in the art (e.g., primer picking software, probe picking software, etc.).

### Recombinant Polypeptide Expression

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Diagnostic Kits

The invention provides kits for assessing LAS risk in environmental samples. In one embodiment, the kit includes a composition containing at least one agent that binds a polypeptide or polynucleotide whose expression is increased in LAS exposed cells. In another embodiment, the invention provides a kit that contains an agent that binds a nucleic acid molecule whose expression is altered upon LAS exposure. In some embodiments, the kit comprises a sterile container which contains the binding agent; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired the kit is provided together with instructions for using the kit to assess LAS risk. The instructions will generally include information about the use of the composition for determining LAS risk. In other embodiments, the instructions include at least one of the following: description of the binding agent; warnings; indications; counter-indications; animal study data; clinical study data; and/or references. The instructions may be printed directly on the container (when present), or as a label applied to the container, or as a separate sheet, pamphlet, card, or folder supplied in or with the container.

### EXAMPLES

### Example 1

### Cytotoxic Effects of LAS on Caco-2 Cells

The Caco-2 cell line is a continuous line of heterogeneous human epithelial colorectal adenocarcinoma cells, which may be cultured under specific conditions to become differentiated and polarized so that they adopt a phenotype that morphologically and functionally resembles the enterocytes lining the small intestine. In particular, when cultured under conditions that give rise to a monolayer, Caco-2 cells may be used as an in vitro model of the human small intestinal mucosa. For example, Caco-2 cells express tight junctions, microvilli, and a number of enzymes and transporters that are characteristic of such enterocytes (e.g., peptidases, esterases, P-glycoprotein, uptake transporters for amino acids, bile acids carboxylic acids, etc.).

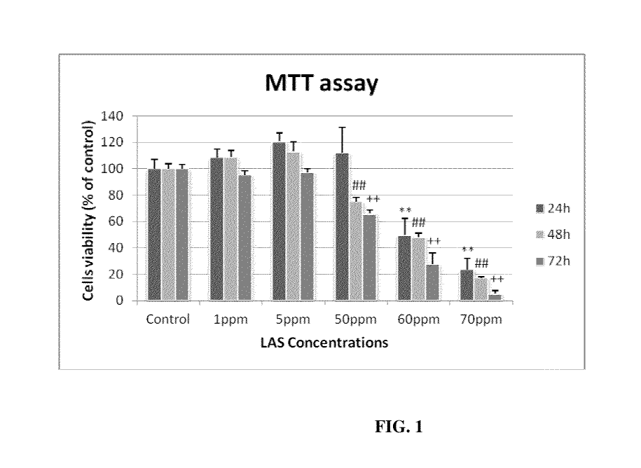

Caco-2 cells were used as a model to assess the cytotoxic effects of LAS. Specifically, Caco-2 cells were treated with concentrations of 0, 1 ppm, 5 ppm, 50 ppm, 60 ppm, and 70 ppm and analyzed by an MTT assay at 24 h, 48 h, and 72 h post-treatment. As shown in FIG. 1, the MTT assay results showed a strong cytotoxic effect of LAS on Caco-2 cells that increased in both a time and dose-dependent manner. In particular, the MTT assay results revealed a time and dose dependent cytotoxicity in which a 50% reduction in Caco-2 cell viability was observed within 24 h of exposure at a LAS concentration of 60 ppm. Consequently, an LAS concentration of 60 ppm was used in all subsequent experiments designed to study LAS cytotoxicity.

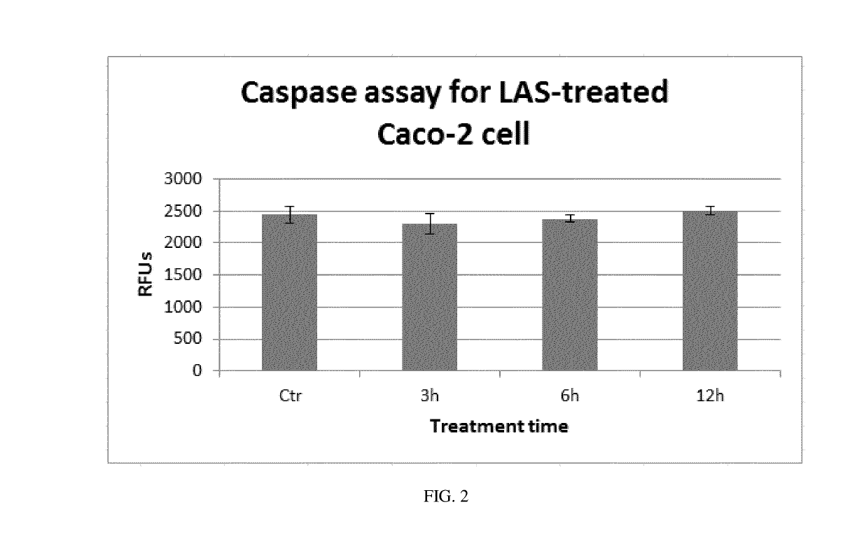

To determine whether the cell death caused by the LAS cytotoxic effect was due to apoptosis, LAS treated Caco-2 cells were analyzed in a caspase assay, as described in greater detail below. As shown in FIG. 2, LAS induced cell death occurs via a non-apoptotic pathway. This was further confirmed by analyzing the LAS treated Caco-2 cells in a DNA fragmentation assay and determining that no DNA fragmentation was observed (data not shown).

### Example 2

### LAS-Induced Effects on Protein Expression in Caco-2 Cells

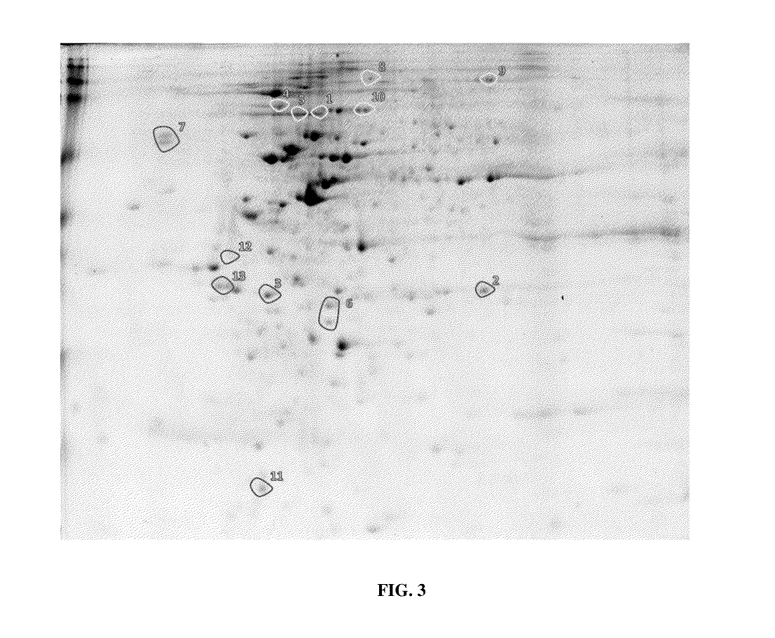

The effect of LAS exposure on protein expression profiles in Caco-2 cells was analyzed by 2D gel electrophoresis. Two populations of Caco-2 cells were analyzed: a first untreated population (i.e., a control population), a second population treated with 5 ppm LAS (i.e., an experimental population with no obvious cytotoxic effect as shown in FIG. 1 of the MTT assay), and a third population treated with 60 ppm LAS (i.e., an experimental population with a significant strong cytotoxic effect that reduces cell viability by about 50%). Total protein was extracted from all three populations 24 hours after treatment, as described in detail below. Alterations in protein expression caused by the LAS treatment were detected as differences in protein expression profiles in the experimental populations relative to the control population. For example, FIG. 3 shows the gel profile of the control population in which the labeled spots represent the proteins that were differentially expressed in the experimental population of Caco-2 cells with LAS. The labeled spots (i.e., the differentially expressed proteins) were chosen based on the results of software analysis designed to assess the respective fold changes of protein levels in the each of the respective spots.

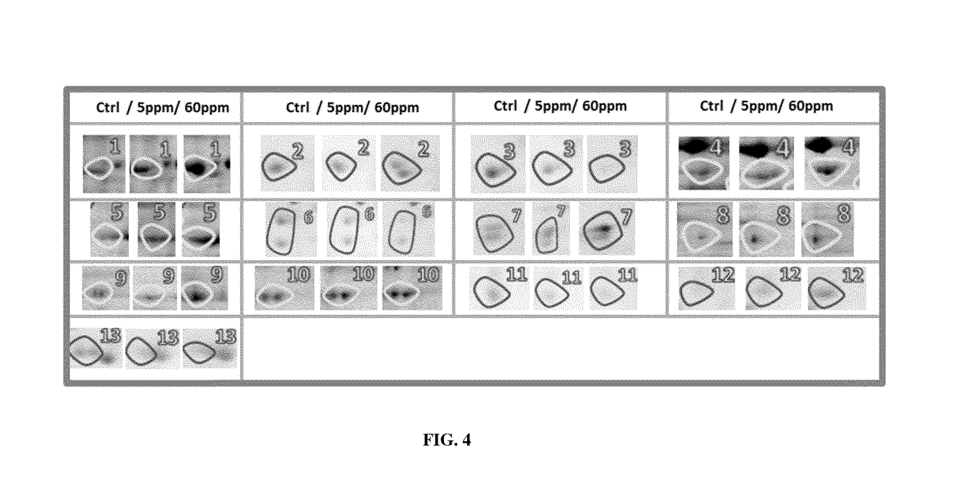

The above-identified proteins derived from each of the LAS-treated, or untreated, populations were compared in a side by side configuration as shown in FIG. 4, which shows images of isolated protein spots obtained from control, 5 ppm LAS treated cell populations, and 60 ppm LAS treated cell populations after gel analysis. As shown in FIG. 4, LAS treatment induced up-regulation of several proteins (e.g., spots 1, 2, 4, 5, 7, 8, 9, 10, and 12), down-regulation of several proteins (e.g., spots 3, 6, 11, and 13), and the creation of new proteins (e.g., the lower band in spot 2).

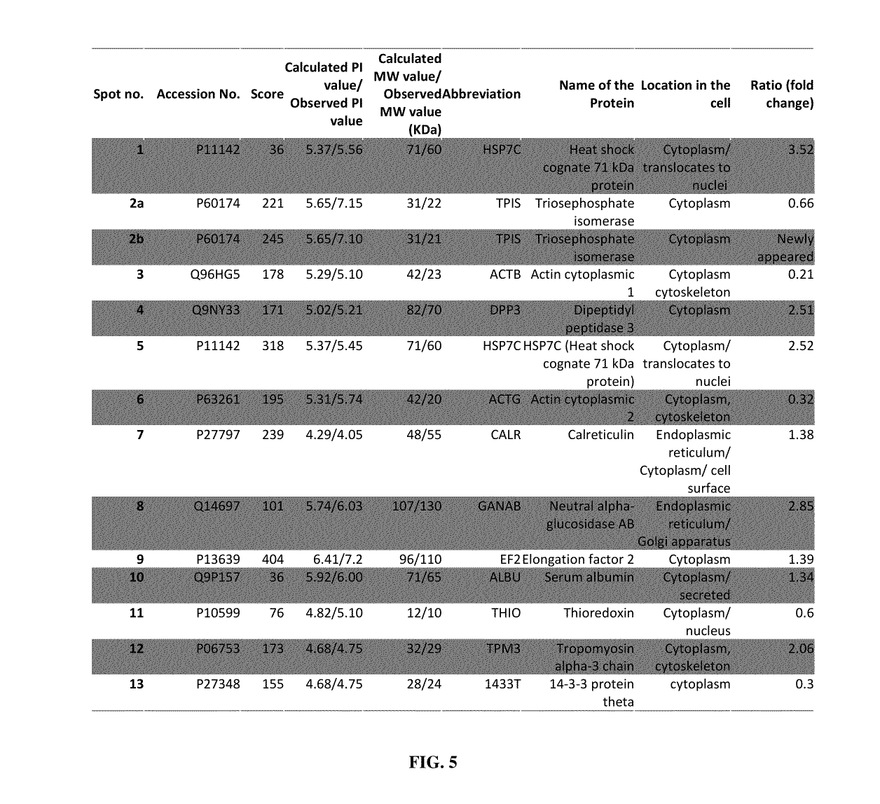

The LAS-induced protein expression changes were compared to the control, and the percent changes were quantitated in terms of fold change of protein expression levels. As shown in FIG. 5, 13 proteins were identified as potential biomarkers for LAS exposure after LC/MS/MS analysis based on their differential expression in control cell populations vs. experimental cell populations. All 13 of the potential LAS biomarkers were subjected to a rigorous investigation possible relationship to cytotoxicity. While the fold change in expression was a central criteria for the initial analysis of LAS biomarker candidates, it was not considered as a criteria for final LAS biomarker selection. Several proteins were not selected as LAS biomarkers their biological function was not considered to be informative with respect to cell cytotoxicity or cell stress (e.g., they may have been related to general metabolism). However, four proteins were identified as candidates to be biomarkers for LAS-induced toxicity: Calreticulin (CALR), Thioredoxin (THIO), Tropomyosin alpha-3 chain (TPM3) and Heat shock cognate 71 kDa protein (HSP7C). These proteins were studied via real-time PCR to investigate their gene expression patterns/profiles. Additionally, they were subjected to different bioassays in order to understand and interpret their roles in LAS cytotoxicity.

### Example 3

### Thioredoxin (THIO) is a Biomarker for LAS-Induced Cytotoxicity

Thioredoxin is a redox-regulating protein, involved in oxidative stress response via, mainly, reactive oxygen species (ROS) scavenging and cytoprotection functions (e.g., Rie Watanabe, Hajime Nakamura, Hiroshi Masutani, Junji Yodoi (2010) Anti-oxidative, anti-cancer and anti-inflammatory actions by thioredoxin 1 and thioredoxin-binding protein-2. Pharmacology & Therapeutics 12: 261-270, hereby incorporated by reference in its entirety for all purposes). THIO plays a role in oxidative stress in several diseases and infections (e.g., Xiang Yang Zhang, Da Chun Chen, Mei Hong Xiu, Fan Wang, Ling Yan Qi, Hong Qiang Sun, Song Chen, Shu Chang He, Gui YingWu, Colin N. Haile, Therese A. Kosten, Lin Lu, Thomas R. Kosten (2009) The novel oxidative stress marker thioredoxin is increased in first-episode schizophrenic patients. Schizophrenia Research 113:151-157; and also Takumi Jikimoto, Yuko Nishikubo, Masahiro Koshiba, Sugayo Kanagawa, Sahoko Morinobu, Akio Morinobu, Ryuichi Saura, Kosaku Mizuno, Shohei Kondo, Shinya Toyokuni, Hajime Nakamura, Junji Yodoi, Shunichi Kumagai (2001) Thioredoxin as a biomarker for oxidative stress in patients with rheumatoid arthritis. Molecular Immunology 38:765-772, each of which is hereby incorporated by reference in its entirety by reference for all purposes).

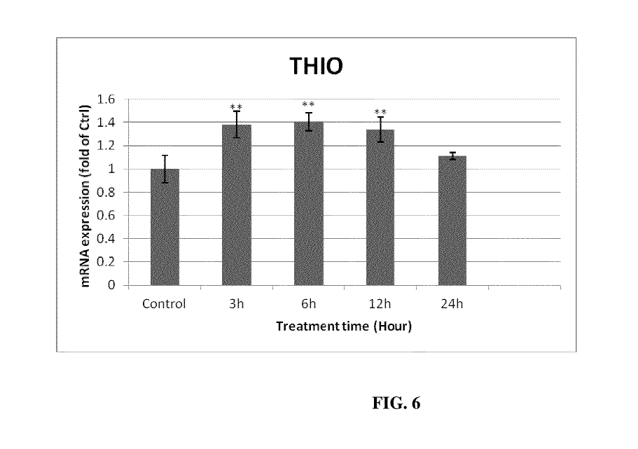

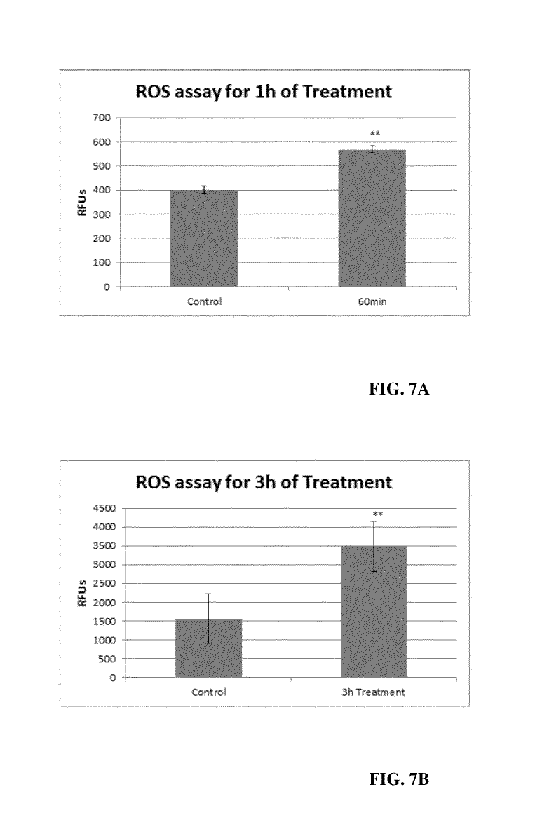

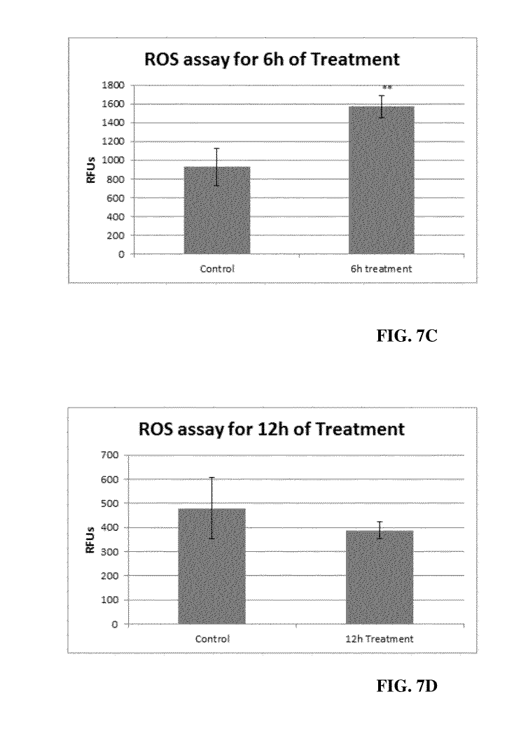

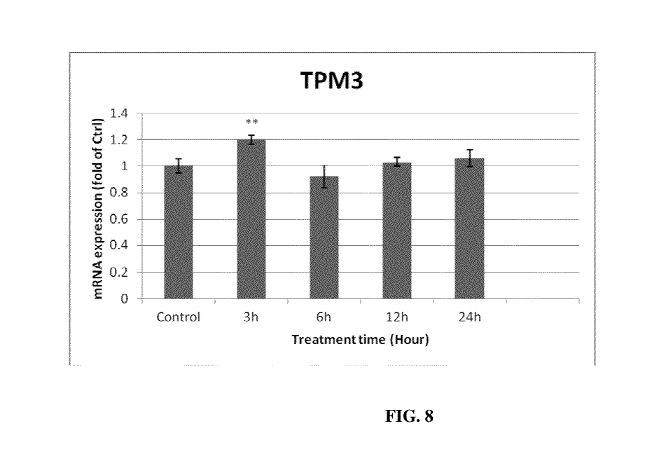

As shown in FIG. 6, THIO overexpression in Caco-2 cells increased 1.4 fold (relative to the control) within the first 3 hours of LAS exposure and remained at a 1.4 fold increased level for the first 6 hours following LAS exposure. Note that in all figures asterisks represent the significance of the difference compared to the control, e.g., * for p value<0.05 and ** for p value<0.01. THIO overexpression decreased to about 1.3 fold (relative to the control) by 12 hours after LAS exposure, and continued to decrease to about 1.1 fold by 24 hours after exposure. Without being bound by any particular theory, it is believed that the increase in THIO protein levels may be correlated with the onset of oxidative stress in LAS-treated Caco-2 cells. To assess this, LAS-treated cells were subjected to an ROS assay at 1 hour, 3 hours, 6 hours, and 12 hours post-treatment. As shown in FIGS. 7A-7B, LAS treatment induced significant ROS production in Caco-2 cells within the first 6 hours after treatment, which returned to baseline levels (or lower) by 12 hours post-treatment. Accordingly, THIO may be an effective oxidative stress response-effector for detecting/analyzing the oxidative stress-inducing effect of LAS, and thus its cytotoxic effect in more general words.

### Example 4

### Tropomyosin Alpha-3 Chain (TPM3) is a Biomarker for LAS-Induced Cytotoxicity

TPM3 is involved in the stabilization of cytoskeletal actin filaments (e.g., Creed S J, Desouza M, Bamburg J R, Gunning P, Stehn J. (2010) Tropomyosin isoform 3 promotes the formation of filopodia by regulating the recruitment of actin-binding proteins to actin filaments. Exp Cell Res. 317(3):249-61, hereby incorporated by reference in its entirety for all purposes), and the cytoskeleton is known to be sensitive to oxidative stress. For example, oxidative stress may induce rearrangement/alteration of actin filaments within the cytoskeleton (e.g., Banan, A.; Choudhary, S.; Zhang, Y.; Keshavarzian, A. (2000) Peroxynitrite-induced nitration & oxidation in cytoskeletal instability & loss of intestinal epithelial barrier function (BF). Gastroenterology 118(4):A803, hereby incorporated by reference in its entirety for all purposes). In the same regard, the ability of epithelial cells to provide barrier functions may be modulated, in part, by the disruption of tight junctions (TJ), which is affected by alteration of the cytoskeleton since TJ proteins are maintained in the TJ structure by the actin filaments of the cytoskeletal (e.g., Hartsock A and Nelson W J. (2008) Adherens and tight junctions: structure, function and connections to the actin cytoskeleton. Biochim Biophys Acta. 1778(3):660-9, hereby incorporated by reference in its entirety for all purposes).

As shown in FIG. 8, TPM3 overexpression in Caco-2 cells increased 1.2 fold (relative to the control) within the first 3 hours of LAS exposure. TPM3 overexpression then dropped to a level slightly below that of the control at 6 hours post-exposure, before increasing to about a 1.05 fold increase relative to the control.

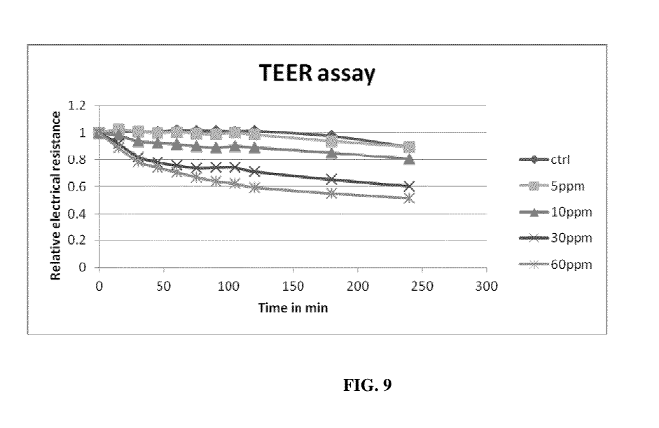

Since TPM3 overexpression would be expected to impact the structure of actin based cytoskeleton features, LAS-treated Caco-2 cells were analyzed in a TEER assay. As shown in FIG. 9, the relative electrical resistance of LAS-treated cells decreased with increasing concentration of LAS, indicating an LAS-induced decrease in the barrier function efficiency of the cells. At higher concentrations of LAS, this decrease in efficiency was observed rapidly post-treatment, e.g., within the first 30 min of exposure. Without being bound by any particular theory, this decrease in barrier function may be caused by the disruption of TJ as a result of cytoskeletal actin filaments alteration.

### Example 5

### Calreticulin (CALR) is a Biomarker for LAS-Induced Cytotoxicity

Calreticulin is a Ca²⁺-binding chaperone, involved in the regulation of intracellular Ca²⁺ homeostasis and endoplasmic reticulum Ca²⁺ storage capacity, and also in autoimmune response (e.g., Pascal Gelebart, Michal Opas, Marek Michalak (2005) Calreticulin, a Ca²⁺-binding chaperone of the endoplasmic reticulum. The International Journal of Biochemistry & Cell Biology 37:260-266, hereby incorporated by reference in its entirety for all purposes). CALR is overexpressed under oxidative stress conditions, and takes part in the cellular response via its cytoprotective effect, in an antioxidant mechanism mediated by the thioredoxin up-regulation (e.g., Lingyun Jia, Mingjiang Xu, Wei Zhen, Xun Shen, Yi Zhu, Wang Wang, and Xian Wang. (2008) Novel anti-oxidative role of calreticulin in protecting A549 human type II alveolar epithelial cells against hypoxic injury. Am J Physiol Cell Physiol 294:C47-C55, hereby incorporated by reference in its entirety for all purposes).

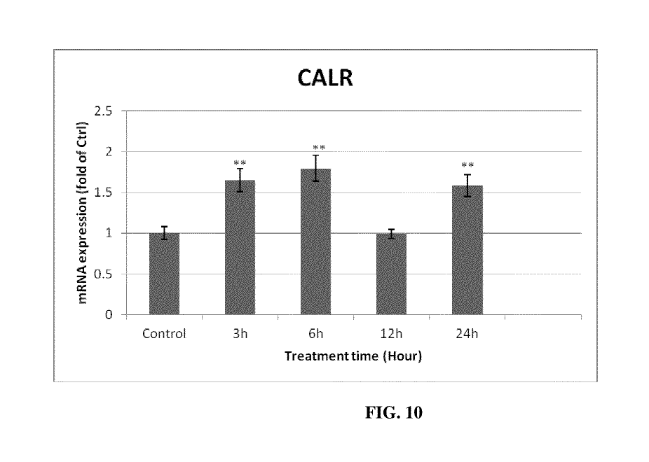

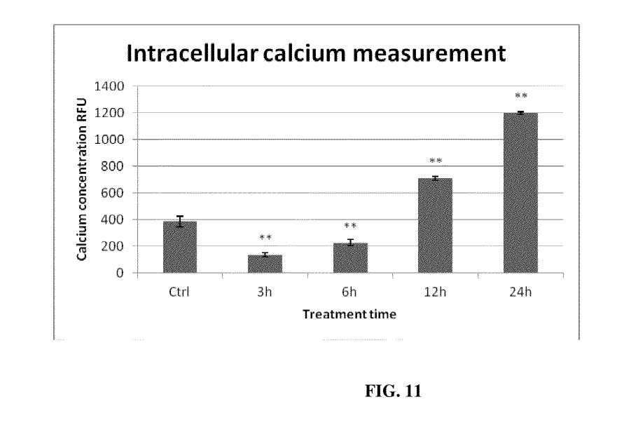

FIG. 10 shows that the expression of CALR in response to LAS treatment increased sharply to about 1.6 fold and about 1.75 fold at 3 and 6 hours post-treatment, respectively. The increase in CALR protein levels was associated with a decrease of the intracellular calcium concentration at 3 and 6 hours post-treatment, as shown in FIG. 11. Without being bound by theory, this may be explained by the Ca²⁺ binding capacity of CALR, as a Ca²⁺ homeostasis regulator. Consistent with this, CALR overexpression matches the observed thioredoxin overexpression in FIG. 6, which suggests that the involvement of CALR in the oxidative stress response may be mediated by the thioredoxin regulation.

As shown in FIG. 10, CALR levels decreased to baseline levels by 12 hours post-treatment. This decrease is associated with an excessive increase of the intracellular calcium concentration, which continued increasing until 24 hours post-treatment. After decreasing to baseline levels, CALR was once again overexpressed at a time point 24 hours after exposure to LAS.

### Example 6

### Heat Shock Cognate 71 kDa Protein (HSP7C) is a Biomarker for LAS-Induced Cytotoxicity

HSP7C, coded by the HSPA8 gene, is a housekeeping chaperone involved in a number of functions, including: chaperone-mediated autophagy, protein translocation across membranes, prevention from protein aggregation under stress conditions, etc. (e.g., Mads Daugaard, Mikkel Rohde, Marja Jaättela (2007) The heat shock protein 70 family: Highly homologous proteins with overlapping and distinct functions. FEBS Letters 581:3702-3710, hereby incorporated by reference in its entirety for all purposes).

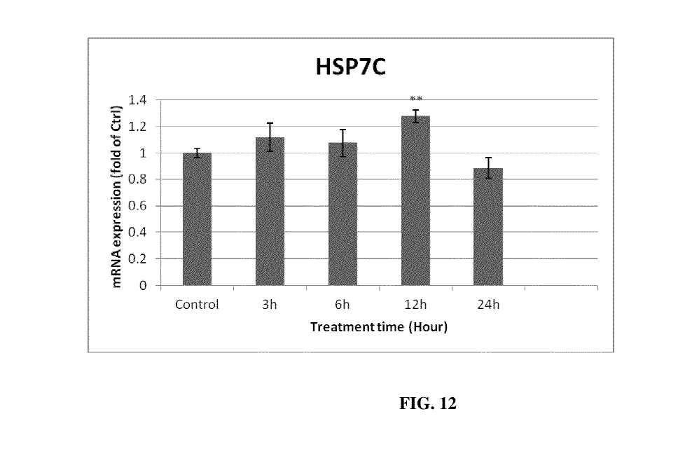

As shown in FIG. 12, HSP7C was significantly overexpressed by 12 hours after exposure to LAS. HSP7C overexpression was associated with the down-regulation of CALR, and thus, with the increase of intracellular free Ca²⁺. Without being bound by theory, the up-regulation of HSP7C may cause intracellular changes that ultimately lead to cell death.

### Example 7

### LAS Biomarkers to Assess the Cytotoxic Effect of LAS in Water Samples

Samples are taken from an aqueous environment of interest (e.g., a stream, river, sewage treatment plant, culvert, etc.) and filtered with a 0.22 μm filters to prepare test water samples. The test water samples are then used to treat Caco-2 cells. After treatment, RNA will be extracted from the treated Caco-2 cells and used for real-time PCR to determine the expression levels of one of more LAS biomarkers (e.g., CALR, THIO, TPM3, and HSP7C), and thus measure the cytotoxic effect of LAS in the water sample.

### Example 8

### Caco-2 Cell Markers Allow a Risk Assessment for the Identification of LAS Effects

According to the techniques herein, the cytotoxic effects of LAS present in a sample solution (e.g., a water sample, soil sample, etc.) are determined by analyzing the protein and/or RNA expression profiles of the above-described LAS biomarkers. For example, RT-PCR of the above-described LAS biomarker genes (e.g., CALR, THIO, TPM3 and HSP7C genes) may be used to assay the RNA expression profiles of one or more of the LAS biomarker genes. It is contemplated within the scope of the disclosure that other methods of determining LAS biomarker expression profiles may be used (e.g., 2D gel electrophoresis, immunoassays, etc.).

Risk assessment is conducted by calculating a PEC/PNEC value, which represents the risk quotient (RQ) used for conventional risk assessment, where PEC represents the concentration of LAS in the tested sample (determined by chemical analysis techniques such as, e.g., HPLC, GC/MS, etc.), and PNEC is a standard value of LAS concentration (the studied compound in general) obtained from guidelines, and represents the highest limit of LAS concentration considered to be without cytotoxic effect. In other words, the PNEC is calculated using exposure to pure compounds, then used as a standard value. The RQ represents the risk calculated when comparing the chemical concentration of the compound (PEC) with the standard PNEC for the pure compound, and thus the RQ will neglect the mixture effect and the interaction of the compound with other contaminants, which in many case may cause a synergetic effect, so that the real risk will be different from the calculated risk. According to the techniques herein, monitoring the effect(s) of the compound instead of only its chemical concentration, and including this effect in the risk calculation, may provide more realistic data for the risk assessment analysis.

To disseminated Caco-2 cells, the sample solution and the control solution are added and the cells may be cultured under certain condition. Total RNA may then be extracted from the Caco-2 cells, and analyzed to determine their Exp value and Ref value by assaying the biomarkers (e.g., TPM3, THIO, HSP7C and CALR) extracted from the cells. The Exp and Ref values may then be applied to the formula below to determine the risk.

$$Risk = \frac{P E C}{P N E C} + ( \frac{Exp 1 - Ref 1}{Ref 1} + \frac{Exp 2 - Ref 2}{Ref 2} + \frac{Exp 3 - Ref 3}{Ref 3} + \frac{Exp 4 - Ref 4}{Ref 4} ) / 4$$

### Where:

PEC: Predicted Environmental Concentration (concentration of LAS in the sample determined by chemical analysis);
PNEC: Predicted No Effect Concentration (standard concentration from guidelines for LAS);
Exp1: Expression level of TPM3 exposed to the water sample;
Ref1: Expression level of TPM3 exposed to LAS solution prepared at same concentration of sample;
Exp2: Expression level of HSP7C exposed to the water sample;
Ref2: Expression level of HSP7C exposed to LAS solution prepared at same concentration of sample;
Exp3: Expression level of CALR exposed to the water sample;
Ref3: Expression level of CALR exposed to LAS solution prepared at same concentration of sample;
Exp4: Expression level of THIO exposed to the water sample; and
Ref4: Expression level of THIO exposed to LAS solution prepared at same concentration of sample.

According to the techniques herein, the concentration of LAS in an unknown sample is determined (e.g., by using HPLC or GC/MS analysis or even colorimetric methods such as Methylene Blue method), and then a pure solution of LAS is prepared at the same time at a pre-determined concentration. In other words, two solutions are prepared: one is the real water sample, and the second is a reference solution of pure LAS.

Two Caco-2 cell populations are individually treated with each solution, respectively. Total RNA is the extracted from each cell population, and measured via real-time PCR to determine the expression levels of the biomarkers of LAS cytotoxic effect in both solutions.

At the same concentration of LAS, if the interaction effects (e.g., between LAS and one or more other compounds present in the complex matrix of sample) are not significant, then the expression levels of the LAS biomarkers will not be significantly different between the two solutions. However, if there are some significant interactions, then the expression levels of biomarkers will display some differences (e.g., up-regulation, down-regulation, new bands, etc.), which will allow assessment of the mixture and the combined effects that may result from the presence of other compounds in the sample, and thus, will provide an idea of the risk LAS will increase or decrease regarding these complex interactions.

If such a difference is identified, it will be calculated in the risk formula and affect the risk value, and thus allow the risk value to be a more real and include the eventual existing interaction effect, that we were not being able to detect with the use of chemical analysis. Consequently, the LAS biomarkers herein provide an advantage in terms of performing more realistic and informative risk assessment.

The results reported above were obtained using the following methods and materials.

### Cell Culture

The human colon adenocarcinoma cell line Caco-2 was kindly provided by Dr. Makoto Shimizu of the University of Tokyo, Japan. Caco-2 cells were cultured in Dulbecco's modified Eagle's medium (DMEM; Sigma) supplemented with 10% fetal calf serum (FCS), 1% nonessential amino acids (NEAA) and 1% Penicillin/Streptomycin (5 mg/ml each), and incubated in a 95% air and 5% CO2 atmosphere at 37° C. The cells were sub-cultured at a split ratio of 1:3 every 2 days.

3-(4,5-Dimethylthiazol-2-yl)-2,5-Diphenyltetrazolium Bromide (MTT) Assay

Cell viability was assessed using a conventional MTT reduction assay. Cells were cultured in 96-well plates and treated with different concentrations of LAS for 24 h, 48 h, and 72 h, respectively. At the respective time points, 10 μl of MTT stock solution (5 mg/ml) was added to the culture medium and incubated for 6 h at 37° C. The formazan was extracted with 100 μl 10% SDS (W/V), and the absorbance was measured with a microliter plate reader at 570 nm wavelength.

### Caspase Assay

Caspase assay was performed using Immunochemistry Technologies, LLC's Apoptosis detection kit, according to the manufacturer's protocol. Briefly, Caco-2 cells were cultured in petri dishes for 24 h, and then 60 ppm of LAS was added to treatment dishes and incubated for 3 h, 6 h and 12 h. After treatment, approximately 290-300 μl of each cell suspension was transferred to sterile tubes (e.g., cell density should be around 1×10⁷ cells/ml). 10 μl of 30×FLICA solution was added directly to the 290-300 μl cell suspensions, and then incubate cells for 1 hour at 37° C. under 5% CO2, protecting the tubes from light. 2 ml of 1× wash buffer was added to each tube. The tubes were then mix and centrifuge cells at <400×g for 5 minutes at room temperature (RT). The supernatant was carefully remove and discard. The cell pellet was resuspended in 1 ml 1× wash buffer, and the cells were centrifuged again at <400×g for 5 minutes at RT. The supernatant was carefully removed and discarded. The cells were resuspended in 400 μl PBS, about 100 μl of the cell suspensions was placed into each of 2 wells of a black microplate. Finally the fluorescence intensity of sulforhodamine (excitation 550 nm, emission 595 nm) was measured and used a fluorescence plate reader.

### DNA Fragmentation Assay

Caco-2 cells were cultured in Petri dishes for 24 hours, and then treated with LAS solution of 60 ppm for 24 hours. At the same time, similar petri dishes was used to culture cells without treatment considered as control. Cells were then harvested by centrifugation, where the supernatant was discarded and the pellet was resuspended in 1 ml of PBS. Genomic DNA was then purified using commercial DNA purification kit from QIAGEN. 1 μg of DNA sample was added to 2 μl of loading buffer (Wako) and then loaded onto a 2% agarose gel. The electrophoresis was carried out at a constant voltage of 100 V for 20 min and the DNA was finally observed under ultraviolet illumination after staining with ethidium bromide.

### Trans-Epithelial Electrical Resistance (TEER) Assay

TEER measurements were obtained by growing the cells at a density of 2·10⁵ cells/cm² in 6.5-mm diameter collagen coated Transwell (0.4 μm PTFE membrane) on 24-well plates. The medium was changed every 2 days, and the cells were cultured for 12 days to establish monolayer integrity. TEER measurements were performed according to the method of Hashimoto et al. (1997). After a 12-day culture period, the cell monolayer was rinsed with PBS. The TEER of the Caco-2 monolayer was then measured using a Millicell-ERS instrument before and after adding various concentrations of LAS, and the effect of the different LAS concentrations on the cells was expressed as the TEER relative to that at zero time.

### Caco-2 Cell Treatment And Protein Extraction

Caco-2 cells were seeded at 2×10⁵ cells/ml density in Petri dishes. After 24 h of incubation in a 5% CO2 humidified incubator at 37° C., cells were treated with either 5 ppm or 6 ppm of LAS for 24 h. The cells were rinsed three times with ice-cold PBS, scraped gently, and collected in PBS. Then, the cell pellet was lysed in 1 mL of lysis buffer containing 7 M urea, 2 M thiourea, 4% w/v CHAPS, 1 mM EDTA, 100 mM DTT, 25 mM spermine base, 1% protease inhibitor cocktail (see, e.g., Han et al. 2010) and 0.1 volume of DNAse I (1 mg/mL)/RNAse (0.25 mg/mL) mixture. DNAse I, RNAse, DTT and Protease inhibitor cocktail were immediately added to the extraction-lysis buffer. The extraction was initially carried out at 4° C. for 45 min to degrade nucleic acid, followed by 1 h shaking at room temperature (Yang et al. 2006). The lysate was then clarified by ultracentrifugation at 46,000 rpm (79660 g) at 15° C. for 60 min. After desalting the protein was extracted using Amicon Ultra centrifugal filters (Ultracel-10K membrane) from Millipore, and the final protein amount was determined using the 2-D Quant kit (GE Healthcare).

### Two-Dimensional Gel Electrophoresis (2-DE):

The first dimension electrophoresis was carried out on an Ettan IPGphor II (GE Healthcare) apparatus. Immobilized pH gradient (IPG) strips (pH 3-10, 24 cm, GE Healthcare) were rehydrated (7 M Urea, 2 M Thiourea, 2% CHAPS, traces of Bromophenol blue, 50 mM DTT and 0.5% IPG buffer, IPG buffer and DTT were added immediately before use) with 350 μg of sample solution. The total volume loaded per strip was 450 μL. The rehydration and separation programs were processed using the following parameters: step 1: 500 Vh, step 2: 750 Vh, step 3: 16.5 KVh, step 4: 27.5 KVh and step 5 was 500 V for 24 h. The proteins were separated according to their isoelectric points. The isoelectrically focused IPG strips were immediately equilibrated for 15 min using equilibration buffer (6 M urea, 50 mM Tris-HCl, pH 8.8, 30% glycerol (w/w), 2% (w/v) SDS, traces of bromophenol blue). The first equilibration was with 1.0% w/v DTT followed by a second equilibration with 2.5% w/v iodoacetamide. The strips were then immersed in 10 mL of electrophoresis buffer for 5 min, and subsequently subjected to a second dimension electrophoresis (255 mm 9 200 mm 9 1 mm) in which the proteins were separated using 12% SDS PAGE with an Ettan DALTSix™ electrophoresis unit (GE Healthcare). The SDS-PAGE was performed at 2 W/gel for 40 min, then 15 W/gel until the dye front reached the bottom of gels. The gels were fixed with 3% ethanol, 0.5% acetate solution, and then stained with CBB for 8 h. After staining, the gels were destained by rinsing with fixing solution. The destained gels were then scanned at 300 dpi resolution, and the images were analyzed with Image Master™ 2D software (ver. 4.9: GE Healthcare). For statistical quantification, three experiments were performed for each experiment. Coomassie blue stained 2-DE gel images were acquired with the image scanner and subsequently subjected to visual assessment to detect changes in protein expression level between different treatments. Spots were expressed as percentages (% vol) of relative volumes by integrating the value of each pixel in the spot area as described previously in our study (see, e.g., Han et al. 2010).

### In-Gel Digestion And Mass Spectrometry

Protein spots of interest were excised from the CBB stained gel, and the excised spots were transferred to Eppendorf tube loaded with 100 μL of 50% ACN/25 mM ammonium bicarbonate solution (1:1). After being decolorized, gel samples were rehydrated with 100 μL of 100% ACN for 5 min and then thoroughly dried in the SpeedVac concentrator (miVac, England) for 5 min. Then, the dried gels were reduced in 100 μL 10 mM DTT/25 mM ammonium bicarbonate with shaking at 56° C. for 1 h, and washed with 100 μL of 25 mM ammonium bicarbonate with shaking at room temperature for 10 min. Reduced gel particles were then alkylated in 100 μL of 55 mM Iocetamide/25 mM ammonium bicarbonate and incubated in the dark for 45 min at room temperature and washed as described previously. After that, gel samples were dehydrated with 100 μL of 100% ACN for 10 min and then thoroughly dried in the SpeedVac concentrator for 5 min. Subsequently, the dried gel particles were rehydrated with 2 μL/sample trypsin in 25 mM ammonium bicarbonate (enzyme ratio 1:50) at 4° C. for 30 min, and then incubated at 37° C. for 15 h. After trypsin digestion, the supernatant was transferred to another tube. The remaining peptide mixture was extracted twice with 50% ACN/5% formic acid at 37° C. for 30 min using 50 μL for the first extraction and 25 μL for the second extraction. Subsequently, the combined solution was concentrated in the SpeedVac to 10 μL and analyzed using LC/MS/MS. The obtained data was used for the identification of proteins using the Mascot database.

### Intracellular ROS Measurement

The determination of intracellular ROS was performed using the OxiSelect™ Intracellular ROS Assay Kit, from CELL BIOLABS, INC., according to the manufacturer's protocol. Briefly, Caco-2 cells were cultured in a 96-well plate for 24 h, and then pre-incubated for 60 min with DCFH-DA. The LAS sample (60 ppm) was then added to the cells. After a different incubation times of, for example, 1 h, 3 h, 6 h, and 12 h, the fluorescence was read using a plate reader at 480 nm/530 nm excitation/emission wavelengths. The ROS content was determined by comparison with the predetermined DCF standard curve.

### Real-Time PCR Analysis

Caco-2 cells treatment and RNA extraction: Caco-2 cells were seeded at 2×10⁵ cells/ml density in Petri dishes. Following overnight incubation in a 5% CO2 humidified incubator at 37° C., the cells were treated with 60 ppm of LAS for 3 h, 6 h, and 12 h. Total RNA was then purified using the ISOGEN kit (Nippon GeneCo Ltd., Japan) following the manufacturer's instructions.

cDNA synthesis: Total RNA was quantified using the Thermo scientific Nanodrop 2000 (USA), and reverse transcription reactions were performed using the Superscript III reverse transcriptase kit (Invitrogen, Carlsbad, Calif.) using 1 μg of total RNA. Briefly, RNA was denatured by incubation at 65° C. for 5 min, with 1 μL oligo (dT) primers, and chilled at 4° C. SuperScript III reverse transcriptase was then added and the reaction mix was incubated at 42° C. for 60 min, and then for 10 min at 70° C. (Han et al. 2010).

Real-time PCR: The expression of TPM3, THIO, CALR and HSP7C, respectively, in treated Caco-2 cells was determined by real-time PCR using glyceraldehyde 3-phosphate dehydrogenase (GAPDH) as an internal positive control. Oligos for TPM3 (Hs01900726_g1), THIO (Hs01555212_g1), CALR (Hs00189032_m1), HSP7C (Hs03045200_g1) and GAPDH (Hs02758991_g1) were inventoried gene expression assays (see Table 5). TaqMan real-time PCR amplification reactions were performed in a 20 μl reaction mixtures containing: 10 μl of TaqMan Universal PCR Master Mix UNG (2×), 9 μl of template cDNA (100 ng μl⁻¹) and 1 μl of the corresponding primer/probe mix, using an AB 7500 fast real-time system (Applied Biosystems). For the amplification, the following cycling conditions were applied: 2 min at 50° C., 10 min at 95° C., and 40 cycles of 15 s at 95° C./1 min at 60° C.

TABLE 5
RT-PCR Oligos

| Primer Name | Gene | Sequence |
|---|---|---|
| TPM3 | tropomyosin 3 | GTGCTTTGTATCAGTCAGTGCTGGA |
| | | (SEQ ID NO: 9) |
| HSP7C | heat shock | AACTGGCTTGATAAGAATCAGACTG |
| | 70 kDa | (SEQ ID NO: 10) |
| | protein 8 | |
| CALR | calreticulin | GCCTGGACCTCTGGCAGGTCAAGTC |
| | | (SEQ ID NO: 11) |
| THIO | thioredoxin | TTTCTTTCATTCCCTCTCTGAAAAG |
| | | (SEQ ID NO: 12) |

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method, comprising:
contacting a population of cells with a sample;
measuring an expression level of one or more linear alkylbenzenesulfonate (LAS) biomarkers in the cell population;
comparing the level of expression of the one or more LAS biomarker to one or more reference values corresponding to the one or more LAS biomarkers; and
determining an LAS risk associated with the sample.

2. The method of claim 1, wherein the population of cells is a population of Caco-2 cells.

3. The method of claim 1, wherein the one or more LAS biomarkers are selected from the group consisting of tropomyosin alpha-3 chain (TPM3), thioredoxin (THIO), heat shock cognate 71 kDa (HSP7C), and calreticulin (CALR).

4. The method of claim 3, wherein the one or more LAS biomarkers is TPM3.

5. The method of claim 3, wherein the one or more LAS biomarkers is THIO.

6. The method of claim 3, wherein the one or more LAS biomarkers is HSP7C.

7. The method of claim 3, wherein the one or more LAS biomarkers is CALR.

8. The method of claim 1, wherein the expression level of the one or more LAS biomarkers corresponds to a mRNA level or a protein level.

9. The method of claim 1, wherein determining an LAS risk further comprises:
calculating the LAS risk according to Formula (I)
$$Risk = \frac{P E C}{P N E C} + \frac{( \begin{matrix}\frac{Exp 1 - Ref 1}{Ref 1} + \frac{Exp 2 - Ref 2}{Ref 2} + \frac{Exp 3 - Ref 3}{Ref 3} + \\ \frac{Exp 4 - Ref 4}{Ref 4}\end{matrix} )}{4} , \qquad Formula ( I )$$
where, PEC is a Predicted Environmental Concentration, PNEC is a Predicted No Effect Concentration; Exp1 is a TPM3 expression level in the cell population; Ref1 is a TPM3 expression level in a standard; Exp2 is an HSP7C expression level in the cell population; Ref2 is a HSP7C expression level in a standard; Exp3 is a CALR expression level in the cell population; Ref3 is a CALR expression level in a standard; Exp4 is a THIO expression level in the cell population; and Ref4 is a THIO expression level in a standard.

10. The method of claim 1, wherein the sample is selected from the group consisting of a water sample, a soil sample, and a sewage sample.

11. A method, comprising:
contacting a population of cells with a sample;
measuring a level of RNA expression of one or more linear alkylbenzenesulfonate (LAS) biomarkers; and
comparing the level of RNA expression of the one or more LAS biomarkers to a reference value for each of the one or more LAS biomarkers to determine presence or absence of an LAS risk in the sample.

12. The method of claim 11, wherein the population of cells is a population of Caco-2 cells.

13. The method of claim 11, wherein the one or more LAS biomarkers are selected from the group consisting of tropomyosin alpha-3 chain (TPM3), thioredoxin (THIO), heat shock cognate 71 kDa (HSP7C), and calreticulin (CALR).

14. The method of claim 11, wherein the one or more LAS biomarkers is TPM3.

15. The method of claim 11, wherein the one or more LAS biomarkers is THIO.

16. The method of claim 11, wherein the one or more LAS biomarkers is HSP7C.

17. The method of claim 11, wherein the one or more LAS biomarkers is CALR.

18. The method of claim 11, wherein determining an LAS risk further comprises:
calculating the LAS risk according to Formula (I)
$$Risk = \frac{P E C}{P N E C} + \frac{( \begin{matrix}\frac{Exp 1 - Ref 1}{Ref 1} + \frac{Exp 2 - Ref 2}{Ref 2} + \frac{Exp 3 - Ref 3}{Ref 3} + \\ \frac{Exp 4 - Ref 4}{Ref 4}\end{matrix} )}{4} , \qquad Formula ( I )$$
where, PEC is a Predicted Environmental Concentration, PNEC is a Predicted No Effect Concentration; Exp1 is a TPM3 expression level in the cell population; Ref1 is a TPM3 expression level in a standard; Exp2 is an HSP7C expression level in the cell population; Ref2 is a HSP7C expression level in a standard; Exp3 is a CALR expression level in the cell population; Ref3 is a CALR expression level in a standard; Exp4 is a THIO expression level in the cell population; and Ref4 is a THIO expression level in a standard.

19. The method of claim 11, wherein the sample is selected from the group consisting of a water sample, a soil sample, and a sewage sample.


SEQUENCE LISTING (12 sequences)

SEQ ID NO: 1 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 2 - DNA, 5891 bp, Homo sapiens
gcggcgtccgtccgtactgcagagccgctgccggagggtcgttttaaagggcccgcgcgttgccgccccctcggcccgccatgctgctatccgtgccgctgctgctcggcctcctcggcctggccgtcgccgagcctgccgtctacttcaaggagcagtttctggacggaggtaacgcctggtcccgcctcgaggccgccccgacgacgcggccggcccccgatcctggatctgcgttgtcgcccgtaattaccgtttagaggtccaacacggtggcctcccgggactagagccgcgggcgatttctcttctgcgtccctggggagcgcggagggcgtagcggcctcccgcggcgggagttagggttagcccgaggatctctgaaggcacccgacgtgtcaaactagaggttggaatggggagtgtcggggatctcctttcctgtccccagcagcttgtggctctcggcagatgtttggtgtggggggggattagcacagccgctctgacctacccctctaatcccccacttagacgggtggacttcccgctggatcgaatccaaacacaagtcagattttggcaaattcgttctcagttccggcaagttctacggtgacgaggagaaagataaaggtaagagcctaggagtgggtgctcagatccgggaggacttcctggcagaagtccttgtctgtacacacacagccgggacagtccccttggaggaggacaggtggaggaagtgggggagtcttctctattctctaagtcgagggtcctcgcgagtcaaggcccaacggtgacctcactaccgtcccgtctcaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtgagggcctgcttcctggtgctgatctctgtcccattagttagagggagacccagaccccattgactttcttaataatgattttttttggaaggggagctaaaagaataagtcccagcaacaatttattgcattatgatcgcagatctaggctgttaatttaatttgcgtgtttgtatatagttatttcccaatcttactaatgaggattttgagttctagagcactgattttttttttttctcctttaaacttaaggctccacccacagcccattcaggacagaatcagggtctgagtttctcttctcagccttgacagacccgagttgaagaaccaggtcttccttttataaagaggggtgagagcctcgagatgatgggtagtctctgactcttaactggatctgcttcacacctaggtcccgacatctgtggccctggcaccaagaaggttcatgtcatcttcaactacaagggcaagaacgtgctgatcaacaaggacatccgttgcaaggtgtgcctgggggtggtggcaaatggctgtcatggggagattcagaggtcagcctcattggggggtggcccccgctcaccttcttccttcttcaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggttggtgtttgggcaggggctctgctctccacattggagggtgtggaagacatctgggccaactctgatctcttcatctaccccccaggactgggacaagcccgagcatatccctgaccctgatgctaagaagcccgaggactgggatgaagagatggacggagagtgggaacccccagtgattcagaaccctgagtacaaggtgagtttggggctctgagcagggctggggctcacagtggggagtgcaccaaccttactcacccttcggtttccttctcccttctgcagggtgagtggaagccccggcagatcgacaacccagattacaagggcacttggatccacccagaaattgacaaccccgagtattctcccgatcccagtatctatgcctatgataactttggcgtgctgggcctggacctctggcaggtgagacttggaggaaaaaggaggatccctggggtacctcaagtgcataagatcacccaagaggaaagggacagggtaggcaccccaggtgagtctgactcaaaaatggtacttcttgtaaacagtacttcctggtctgtccctgtgaagtcctcacagcaacccctttaaggttatacttgctgtgcaccaagtacttccccaagtacttttatgcaaatcaacttctttacccccaaagacctagaaggtggtcaggtaacccagttagttagctggggctgggcacagtggctcacccttacaatcacggtactttgggaggctgagacagaggattgcttgaggccaggagttacacaactcaacctagcttggcaacacagcgaggagaccctatctctacaaaaaaaatttttttttttgagacagagtttcactcttgttgctgaggctggagtgcaatggcacgatctcagctcactgcgccctccgtctcctggtttcaagcgattctcctgcctcagcctccggagtagctgggattacaggcatgtgctactatggatgccaggctaatttttttttttttttttttttttgagaccgtgccttgctctgtcgcccaggctggagtgcagtggtgtgatctctgctcactgcaagctccgcacgaccccccaggttcactccattcttctgcctcagggtcccgagtaactgggactacaggcaccccccaccatgcctggctaatttttttgtatttttttttttagtacagacatggtttcaccgtgttagccaggatggtctccatctcctgacctcatgaaccacccaccttggcctcccaaagtgctgggattacaggcgtgagccacctcacccagcctttttgtagagacagggcttcatgttgcccaggttggtctcgaactcctggcctcaggtcatctgcccgcctcggcctcccaaagtgctgggattacaagggttagccaccatgcctagcctctacaaaaactttaaaaattggcgagatgtcatgcatacctgtagtcccaactaccaaggaagaaggatgatcacttgagcctggggcatcgaggctgcagtgagccatgattatgtcactgcactccagcctcggtgacagagtgagaccctctcaaaaaaagttgggacttggccggacacagtggctcacacctgtaatcccagcactttgggaggccaaggcgggtggatcacaaggtcaggagatggagaccatcctggctaacatggtgaatgaaaccccatctctagtaaaaatacaaaaaatttgccaggtgtggtggtgggcgcctgtagtcccagctactcgggaggctgaggcaaaaggatgacgtgaacccgggaggcggagcttgcagtgagctgagatcatgccattgcactccagcctgggtgatagcgagactctgtcccaaaaaaaaaaaaaaatgctgggactgaatttttgtctgttttggtcactgaaataccttctgtgcccaagacagttctggcatgtagtaggtacctgaaaaatacctgaataagagagtgagaaacaagaaacaggtgcagagaactgaagtcagtggcccaaggtcatgggggtaggaaaccacaaagctggggtttgaacctgggcagtacagcacctgagtctctccatctttttttttttttttttttaagacagagtcttgctctgtcacccaggttggagtgcagtggcttgatctcggctcactgcagcctctgccttccaggttcaagtgattctcatgcctcatcctctcgagcagctggaattacaggcatgcgccacgacgctgggcttttttttttttgagatggaatttcactcttgttgcccaggctggagtgcaatgatgcaatctcggcggctcaccacaacctctgcatcccagattcaagcgattctcctgcctcggcctcctgagtagctgggattacagggatgcgccatcacagaccccgggctaattttttttagtagagacagagtttcactatgttgcccaggttggtctcgaactcctggcctcaagtgatccgttcgccatgacctcccaaagtgctgggattacaggcatgagcccgtcccgtccctggctgtctctccatctttccatcttttttttttttttttttttttttggagatggagtctcactctgtcacccaggctggagtgcagtggcacgatcttggctcactgcaagctccgcctcctgggttcacatcattctcctgtctcagcctcccaaatagctgggactacaggcacttgccaccacgcctggctgattttttgtatttttagtagagacggggtttcaccgtgttagccagggtggtctcgatctcctgacctcgtgatccgcccaccttggcctctgggcgaggattacaggcgtgatccacctcacctggcctctccatctttttaactgcagtgtcagcggtgttccttgtcttctctgcagatgcaggcagcagaatatagtggttataggaacacaggtggaaaccctgtccaaagcaagggctatcgggtatcacctctgaccatccttcccattcatcctccaggtcaagtctggcaccatctttgacaacttcctcatcaccaacgatgaggcatacgctgaggagtttggcaacgagacgtggggcgtaacaaaggtgaggcctggtcctggtcctgatgtcgggggcgggcagggctggcagggggcaaggccctgaggtgtgtgctctgcctgcaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtagagaggcctgcctccagggctggactgaggcctgagcgctcctgccgcagagctggccgcgccaaataatgtctctgtgagactcgagaactttcatttttttccaggctggttcggatttggggtggattttggttttgttcccctcctccactctcccccaccccctccccgcccttttttttttttttttttaaactggtattttatctttgattctccttcagccctcacccctggttctcatctttcttgatcaacatcttttcttgcctctgtccccttctctcatctcttagctcccctccaacctggggggcagtggtgtggagaagccacaggcctgagatttcatctgctctccttcctggagcccagaggagggcagcagaagggggtggtgtctccaaccccccagcactgaggaagaacggggctcttctcatttcacccctccctttctcccctgcccccaggactgggccacttctgggtggggcagtgggtcccagattggctcacactgagaatgtaagaactacaaacaaaatttctattaaattaaattttgtgtctcc

SEQ ID NO: 3 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD

SEQ ID NO: 4 - DNA, 4702 bp, Homo sapiens
ccttctggaaggttctaagatagggtataagaggcagggtggcgggcggaaaccggtctcattgaactcgcctgcagctcttgggttttttgtggcttccttcgttattggagccaggcctacaccccaggtaaaacctctgctcaagagttgggttgtgggtctgggagcgtgcagcctccacacaggcctgttgggcttgctgaggcttgggggttctgagaatctcgtcgaggcgagtgtgcggctccttctaccggcttaaagggcctcagttttcggtgggatggcagcggtatttggttgcagccggcaggacggaaatgtagggagtgggccgcagtggccccaggggaggctgggagacgcccggcggccgcgtggcgggggagggttgctgcatcggtttgcctggcgcgcggggaagtggagccagcgttttctttcacccagttccctgcttagtccagtcccaccgtggttcttcagagctgttcttggcgtgcttccagtatgggggtacattccggagtagttaaaagcccgttgactcccgggggcactggcacctggcgagggaggggaacagacagtgctcagttcggggtaagaccacgtgttgagcaacgccccacgccgtctgggtagatgggtccttcatctagggcgtgctctgctgcggttggcacggcaacctggactgcagcactagttctggacctcgcgcgtgcttagacaggaggtgatgggcactattacctcttggcagtggccatacgtttttcctggttaagtgttctgttaagggatgagggaaatattttgattaattgaatttttaaaccagatttttctttttttcagcaaccatgtccaagggacctgcagttggtattgatcttggcaccacctactcttgtgtgggtgttttccagcacggaaaagtcgagataattgccaatgatcagggaaaccgaaccactccaagctatgtcgcctttacggacactgaacggttgatcggtgatgccgcaaagaatcaagttgcaatgaaccccaccaacacagtttttggtgagttcctaattttaaatgacagaacaaatataacagggctaggaagcacaaaagtttatgaaacgtgaggagggaactttttgattttagaaaaactgagctgagagacttgttatcaagtctgttataaaacaggttgtagaaacctttcaggctgaaatctggataacgtaggaggttgaagtttgaacctttgctacctatatggtagttgaattcacctacctatgaactgttaggtatttgagtaatcatggacttgagttttatcagaagagctatgaaattgaaagtgttttcatttgacaccttttacagatgccaaacgtctgattggacgcagatttgatgatgctgttgtccagtctgatatgaaacattggccctttatggtggtgaatgatgctggcaggcccaaggtccaagtagaatacaagggagagaccaaaagcttctatccagaggaggtgtcttctatggttctgacaaagatgaaggaaattgcagaagcctaccttgggaaggtgaggttggtttttcagtatggggtgcattccggagtagttaaaagcccgatgactcccgggggcactggcacctggcgagggaggggaacagatggggctcagctcagggttaagaccacgtgcccaacagtgccctaggctctctaggtagatgggtctgtcaacaccagaaaccagtgaatcttgacaattacacagtaatttacattttggtggggggggtgctccagctgttctttcaccagcattaatccatttgctggagtttgcatatatgtaagtataatagttaccaatctgtggtcttttccttattcctagactgttaccaatgctgtggtcacagtgccagcttactttaatgactctcagcgtcaggctaccaaagatgctggaactattgctggtctcaatgtacttagaattattaatgagccaactgctgctgctattgcttacggcttagacaaaaaggtatgtaccatttgtgatgcaagttcggattattttaagattaatttgatccatcgtaaatttaaatgagattgtttttaacggcaggttggagcagaaagaaacgtgctcatctttgacctgggaggtggcacttttgatgtgtcaatcctcactattgaggatggaatctttgaggtcaagtctacagctggagacacccacttgggtggagaagattttgacaaccgaatggtcaaccattttattgctgagtttaagcgcaagcataagaaggacatcagtgagaacaagagagctgtaagacgcctccgtactgcttgtgaacgtgctaagcgtaccctctcttccagcacccaggccagtattgagatcgattctctctatgaaggaatcgacttctatacctccattacccgtgcccgatttgaagaactgaatgctgacctgttccgtggcaccctggacccagtagagaaagcccttcgagatgccaaactagacaagtcacagattcatgatattgtcctggttggtggttctactcgtatccccaagattcagaagcttctccaagacttcttcaatggaaaagaactgaataagagcatcaaccctgatgaagctgttgcttatggtgcaggtaacaatggtatctcaattaaccctaaaggcaggcaggcccaaggtgactcgctgtgatgagtgattgttaaacattcgtagtttccaccaaaagcttggctaatgatggcaacaccttccttggatgtctgagcgagtgatagttaaaacaggagctatgtactgggttttcttttaacttcttttaacgttaactttttgtttgctagctgtccaggcagccatcttgtctggagacaagtctgagaatgttcaagatttgctgctcttggatgtcactcctctttcccttggtattgaaactgctggtggagtcatgactgtcctcatcaagcgtaataccaccattcctaccaagcagacacagaccttcactacctattctgacaaccagcctggtgtgcttattcaggtatgtttctgtacttctcttgtttggcttactgataacagataaagggaagtcttgactgactcgctatgatgatggattccaaaaccattcgtagtttccaccagaaagtcttatgttggccagttccttccttggatgtttgagcgaccattcttccttagcaggaccctagcactgtcacagacctggagtccattgtagtaatttgttttatttcctaccaaggtttatgaaggcgagcgtgccatgacaaaggataacaacctgcttggcaagtttgaactcacaggcatacctcctgcaccccgaggtgttcctcagattgaagtcacttttgacattgatgccaatggtatactcaatgtctctgctgtggacaagagtacgggaaaagagaacaagattactatcactaatgacaagggtaaggaggcactgtcatctggtcttgacagggataatggtatttcaattgagttactggtgcctaagggcgtctagctaagagaaactagagttacacatacacaggtaatttaaggcttttacttagagttaatttctttcctaggccgtttgagcaaggaagacattgaacgtatggtccaggaagctgagaagtacaaagctgaagatgagaagcagagggacaaggtgtcatccaagaattcacttgagtcctatgccttcaacatgaaagcaactgttgaagatgagaaacttcaaggcaagattaacgatgaggacaaacagaagattctggacaagtgtaatgaaattatcaactggcttgataagaatcaggtttgtgttttttttttttttttttcctcccccactcaatggaggggaaggggatggtaaaccaagcttgagctggatttcagtgtagggtcacaatgatgaatggtccaaaacattcgcggtttccaccagaattcaaggtgttggcaactaccttccttggatgtctgagtgacccaagatgttaaggaagaataaggccctattttaatgttggtagtggccctcttgtaagagtttgcgccagacttttagtatcagattgcgtcagggagaaagaagggttattaacattaaaagaacttgcagtaattcctttttctcttcctcagactgctgagaaggaagaatttgaacatcaacagaaagagctggagaaagtttgcaaccccatcatcaccaagctgtaccagagtgcaggaggcatgccaggaggaatgcctgggggatttcctggtggtggagctcctccctctggtggtgcttcctcagggcccaccattgaagaggttgattaagccaaccaagtgtagatgtagcattgttccacacatttaaaacatttgaaggacctaaattcgtagcaaattctgtggcagttttaaaaagttaagctgctatagtaagttactgggcattctcaatacttgaatatggaacatatgcacaggggaaggaaataacattgcactttataaacactgtattgtaagtggaaaatgcaatgtcttaaataaaactatttaaaattggcaccata

SEQ ID NO: 5 - protein, 105 aa, Homo sapiens
MVKQIESKTAFQEALDAAGDKLVVVDFSATWCGPCKMIKPFFHSLSEKYSNVIFLEVDVDDCQDVASECEVKCMPTFQFFKKGQKVGEFSGANKEKLEATINELV

SEQ ID NO: 6 - DNA, 12829 bp, Homo sapiens
ctcgcaggctccaggggcggggcgtggccggggcgcagcgacgggcgcggaggtccggccgggcgcgcgcgcccccgccacacgcacgccgggcgtgccagtttataaagggagagagcaagcagcgagtcttgaagctctgtttggtgctttggatccatttccatcggtccttacagccgctcgtcagactccagcagccaagatggtgaagcagatcgagagcaaggtacgcgctaccggggaaggccagggtgccggcgccgcgcgcggcctctgtaactggggaaggcggtggcgggaggtggggaaggcggtggcgggaggtgcggaggccgcccctccgcatcgccaggggaaagggacgcggcgtctcggcctgggactgcgggaagcagcggcctgggcgcgcccgaggcggtggagcctgccctggaggaagggaggagaaggacgagggtcccctggagggcggagtggcggtgcccagcgtttctcgcaccctgttcctcgggggattgcacgcacgcggggagcgtccgggggatgtgagagcgcagacagcgtgaggagtccccacgctgcgcctcctgcaccctcccgtccgggcagccccgactggaggaagatgagggaatggaaggggtccgcccttggccccccatctgtatccagattcaggccccaggcaaggatagggagggcccttgcagaaggcacgggtcggtggccgccgctgcctttccgtatgtgaagtgatccacccgcagcgggggtagtgatctccctttgggagcgggtctaggccggagacccccgcctgcctccacccatgcccgaccccaaaggtgacgcgtgctgtatccgcactaagggggcggattgcggctggagaccccctggcacgtgcaggtctgtccaggaggcccgagggccccaggtgaccgcgaggaagtgaggtccgggccgcgcccacgggactcctgtggcgcagggcgcgtttccggcagcagtggctttggaatgactgagtccccaaggttgggcccgggggcctcggctgccctgcccgtccatgattcaccctcagtcggtgggttttgctggagccagggttcctcctgggagcagccgcgccctgctgcctgctcgccgacctatcggtatcccgatcgttgttttgtcctcttaaaaatgcccaaggcgaaacagccttcccatgtttgaaagttattgcaagcctaaaaccttgtagactgggaaacccagagcctaacgcgcagtgtctagtccaatgtagccactccagaaatatttgttaaatgcagcgtcagaaaagtgagtggaggaaattgatactgctcgaacggtagaagacccctcgccagcgcctaccctgcgattacccctccctacctgcgggaagcagaggagggcgggtcctcgcccgcctcgggtgccctgacctgtttggtgccgggtgggcttcggaaacagaagtgtgtctgcaatgtgtccccgatccttttgttcctttgattattattgactctcagtgttttttcctcatatgttgattgccactgtcatcttttatcttcctctcaatcagttttttcttagtgggattctcattttagcagccctcatgtgttgaaaagatccttagtagtgaattgtctttcatatactttttttccaagcacctattgtgtgacaaattattaatccattcctggggaagggagtggggctgggattctgttctccagggtctggcaacctcagtataacccaactgctaagaaccccctccactgagccagaagacctttgagtggtctatgttagttgtcccaaaatccagacactacaaacaaagttgattaggatttctggagcacacagtttagtcctcccagttgtcagagcatgtcagagcaccttcctcctctaccagtgacaaaggtgtacaagggtgacaggaactttaaaaaaagcactacagcctggggcccaaaggccctgataatcaattaatcctcaaaataacaatccaaagtcattgatcgaaagttacactaatttgattgttatttgtctgttagtttgtttttcgagatggagttttgcccttgttgccctggctggagtacagtggcgcgatctcggcccactgcaacctccacctcctgagttcaagcgattctcttgcctcagcctcctgcacagctgggattacaggcatgcgctgccaaaatgcccagtaattttgtatttttagtagagatggggtttcaccatgttggtcaggtttgtctcgaactcctgacttcaggtgatccaccaacctcaggctcccaaagtgctaggattacaggcgtgaaccaccacgcccagcctgttatttgtaaatgttgaatacatgttacattttcatcctaatgggctaaatttgcaccatttgccattcagaacaattctgtttctgaggtactctgttggtgctttagggccaactgggatctatttcagagaggaatggaataattgactgtaaatgtgatgaggaagaaataaacacttttaaaaaaaatgacacctaccatttattgaactcccatctacaaggcacttggctaagtacttcagaaaccactcacacttattaccctcagagtaggtatgttgaggcaacgagatcttagactcttgctcctatttaccccaactacactgttctgcttcccccagattattggtgtcagtgatggagacatttattaatcctgttagtttctgggagctagaaattgtgatttcttcttagtaatacaatcttgaataattttcaagctgatacccgtttagaagtatcagaagagaatttgtacatgaagcctgcacatacgtggggtgtaactcatgttcagttaggctaaaagttattgttgcgtgcctcttttcagaattttaggtacttgtgcttaaatttgattcagaactgttttggaaaagccttgagtatgtttgaaataccttccctcttgaaagtaatctcaagtttttaataagggttaatcatgttaaaaaaacaaaaatgtctattcaaccagacattggcatttcttgaccttttttcctgtcttacctggatcttgcaataaaggatgcctggtttaactttcttgaaaatcacattagggaaggctttgaatgaaattgatctggaacaataagtgatgatttggaaaaacaattgctatacttctatgtaccctgctgcagctctccccatgtctccacctctagaggtggggttcagggatttgcataactaaaaaatttatgaaagtgttgtcctacctttctcaggaacaccatttgtgaattattttcccaaaaacgaggtagaaattagaaatctagagaagtaactattagtacatgaggtcatattagtgttttcttgttgggtttttttttgtttatttggttttttatcttatggttttttatttatttgatttctttctttacgagacctcttgtggcggtggggggcggggaatgttcatttttttttaaacctatttgaccagcattgtttccttgaagaaaacctagatttcagatacagatgtttatgttttgatttatcttaattgctctggtttggtttttgggtttggtcagcactaacgtactaatgtggttaaaatgagtcctttgttttgggaggccaaggcgggtggatcacttcaggtcaggagttcaagaccagcctggtcaacatggcgaaactctgtctctactaaaaatacaaaaattagctgggactggtggcagaggcttgtaatcccagctactcaggaggctgaggcaagagaatcacttgaaccccggaggcaaaggttactgtgagccgagatcaggcctttgcactccagcctgggcaacaagtgaaactccgtctcaaaaacaaaacaaaacaaaaatgagtccttggtaactagaatattcggttcccagggttacagtatctagatagtaaataattcagggaagttagtggtaagagatttcttgatcatttctactgagaattttatttaacaagcattccttatgaaaaataatatctatgaaaaatttccttcatgaggaacgaaaactttcatttaatgaatgacaagggtatagttttaaaataaagggcaaaaatcaaaggttggtaaacgtgtgatctcagctctggaaaccccattatgcttatgtcaacggtgatgtctgagtgttgaggtttgggaaaggtgagtttccttgacttttcaaaaaattttagattttcgtatggtccaccatagacaaatgagtttaatcaaaagtcatagcttttttttttttttttttttttgcgacagagtctccgtctattgcccaggatggagtgcagtggcacaattttggctcactgcaacctccgcctcctgagttcaagctattctcctgcctcagcctcctgagtggctgggactacaggcatatgccaccacgcccagctagtttctgtatttttagtagagacagcatttcaccatattggccaggctggtctcgaactcctgacctagtgatccgcccacctcggcctcccaaagtgctgagattacaggtgtgagccaccatgcccagccaacttttatctttaagtaacttgtgatgtttcaattgcaaaatcctatgcctttgtgacttcaagtgacccctttcataatccataagtgtttaatgaatgtctaccatatacctagccttgacatggaaacatttttaatacaaatgtctatttttattttccttttgtttggtgtagagaaaaaatagccagttcacaatattttataaaatagttatgaagagaatgtcagtatactctacacatatcttgtttcatcttatcaagtaacactaccaacaatgtatagaatttcttcaaactgagttttatttggcttgtttggggatttttttttttttttttttttttttttggctaaaaagtaggtcctgaaaggaggacctccagaatgtgctttgtgtcattgtgtcgagtctttcttttgaaggtttaatatttaactatttatttaatataagcttttcttttgctgttagactgcttttcaggaagccttggacgctgcaggtgataaacttgtagtagttgacttctcagccacgtggtgtgggccttgcaaaatgatcaagcctttctttcatgtgagtattaaacaatgtctgctttgtaagagatttgtgttttttgagttggtggtcacagtggtaggaaagaaagacagttaaaggattttggtttcggtggggggatttctttggctggatctttggtctaaaagtagtagtataacaaataatttaggtttgatacatgtagcccattgaaaacaaattttagaagttaattttgtcttaaatagttctttttttccccacattgaaacatgggccttatttgaaatcccagcctcagaatttgatatgccaagctgttttatactaagaaaaatttgatttagagaaaatttatgtctcttagatctatgtctccaaagatctaaatttttggatctttaattagtctctacttttattaagtttccatttaagaagcttgggtatgttgattgccattacctagttctaaatctttttggatttttcattttaaattttccagtccctctctgaaaagtattccaacgtgatattccttgaagtagatgtggatgactgtcaggtatgtagctggaaatatgagatactgctgagcttttcacattggcctttttctctgaattgcacagtgcttttttccataaatatgtcaaataattctagaactgtaatcctatctaaaaagttctatctcagaagagcaggcaagttaggagcttaatcctagctatcgggagctgtatatcacatcctaaagtaaacaaaaataaatgagtgagacttctgaatcttatcggccacccacctttctaaaaccctacattctactttacactctgagatgtgcaataaatggagattgaatttagctatgatcattacatccataggcttgatggagtcaccaaattatgagaccgcttgtagggctctttgtgaacttgcagtagcatgagaacctgcatttgcaagcctattctagtcttggttgattttagtcaattagaaaccacaaatgttttaacaaataaacaccaaggtacctgagagaataatttggaagaaattccagggttggttgtatttaacaaatacttgttttgcactaggtatataccaggcactgttctgggtggtttttaagtatcagttcatttaatcctgagtgctgttatcatccccattttatagatgagaaaactgaaacacagaggttgttcatgaagtttcagtgagtatgtggctgaactaggatttaaaatgaagtggtctggcttcccagcccttgaccttaagcactacccatcggaggatgctctgtcttgtgggtgtagatcgggtgcttagcacatgaccacagacctaggagagcgggttgaggaggtatcacttcggggccctttacagatatgtgagcattttcacttagccctagtggagaaggaaaggcgatgggggaagggtgcagtgtggcaacagaggcgctggacctggcttccagtcctggctcactagcatctgcttaggccagtcactcctcttccttgagccttaagacctgccccatcaacctcccaaggttgcttattcattgagcaaacatggaatatccaataaagggtgaagggtcacttaaaacaggcatatggcagtgctctctaaacatgggagggcgcaacaaccccagattgtgtattcttagccagtttttgactctgtgccttgggcaacccctgccttggcttgtgctgtcttctccatctggcctgtcctttcctttcctacctgactaactccttgtcatgagcttcaccccttctccacttaccgccttgtgtgccctaagtacccagtgaatcttggcaattattataatgatctttatgtctgtcctttaccattagtctcagtagattcctaggatcagagaccctgtcttaattcacgttggttgccccttcacctagcacacctgccttgcatgtagtataggtgtggaatgaatgaatgatgaatgtgatatggttgttaagttactattctagatgtgtcccagagttgtttttttttttttaaaaagagtgtaattgcatttttgtgaaaaatccttatcccttgttttaatcaaacttagtcttattaaggtcaatttagctaggggaaaattgcacctggaatagagaaattctaactgccactgatcctatcagatagcaacttgattttttttttttttttttttttttttttttttttgagatggagttcactctgtcacctggggtggagtgcagtggcatgatctcggctcactgcaacctctgcctcccgggtttaagcaatcctctgtctcagcctcctgggtagctgggattacaggcatgcaccaccatgcctggctaattttgtatttttacaaaattaaaaccccagtagagacggggtttcaccatgttggtcaggctggtctcgaactcctgacttcaggtgatccacccacctcggcctcccaaagtgctgggattatccaccacgcccggccttgatttttatttgaaagcaataataggtgccagatgccatgataagccctttgcatgcactatgtcatttaatcctcacgataactatacgagtattttttattagcaccctcattgaacaggtaatggcactgcagcacagaaggtaaagtcagtctcttgaggcagaccaatgcaccatactgtactgaggacaggtcttcttactgcctttaggaagtacagtcatgcatcacttaatgatgaggatactttctgagaaatgttaggcaattttgttgtgcacacatagagtgtacttacacaacctagatggcatagcctcctgtacacctaggctatgtggtaaagcctgttgctcctaggccacaaacaggtaaggcatgttactgtactgaatactgtaggcagttataacacaacagtaagtatttgtgtatctaaacatagaaaaggtatggtgaaaacatgatatgaaagattaaaaaatggtatgcctttataaggtacttgccataaacgggacttgcaggactgagaggtgctctgggcgagtcagtgagtgggtggtgagtgaatgtgaaggcctagaacctgtagacgttataaacactgtatgcttacaaattttatttttaaaatttcttttttcagcaataaatttattgtaacttttttactttatagtttttttatttttttaactctttaactcttgtaataacacttagcttaaaacacgaatgcattgtacagctctacaaaaatattttctttatatccttagtctataagctttttttaaaaagactttttaaactttttgttacaaactaagattcaaacacatacattagcctagaccaacacagggtcaggatcatcagtatcactgtctcccatccccacatcttgtctcacagaaaggtcttcagggacagtaacatgcatggacctttcatctcctatgataacagtgccttctcctggaatgcttcctgaagggcctgcctgagcctgttgtatagtaactgtctttttaaaaaaataagtaggagtacactctaaattaataatgaaattaaagtaaatacaaaaaccagtaacgtgggtgtttattatcaagtagtatatactgtccataattgtagtgatatgcttttttaagtgaaagcaagtttattaagaaagtaaaggaacaaaagaatggctattccgcaggtaaagcagtctgtagtggtatactttgtatgtaattgcagcgcagatttgtttgcaccagctaatgcgatgggctatgacattaacccatcactaggtgagaggaatttttcaacttcattataatcttatgggaccaccacatatatgcaatctgttgtcgatctaaatgttatatggtgcattactataggtgtgcaaagcactcgaggacttccgtatgacagagctcctccttcatgtctgcttggtgcaccctgatcaccctgaatgtatcttttttttttttttttttgagacagagtctcactctgtcacccaggctggagtgcagtggtaccatctctgctcactgcaacctccacctcccgggttcaagcgattcttctgcctcagcctcctgagtagctgggactacaggcagccgctaccacaccaggctaaatttcaactttttagtagagacagcattttgccatgttggccaggctggtctcgaactcctcacctcaagtgatccacccgtcttggcctcccaaagtgctgggattagaggcatgagctaccgtgcctggcctgaaagtgtcttttaaaaccttgaagtgaccctctgacaaactgaggaactttaactttgcctccatagattgatagaaaagtatgagtagtagcccttttgaaaatgatagaccaaccttatttctctgacagccaacagggttatgatacttattttataaatggtaacctccctctgacccttacttggagtgagttttcaatagtatgcattcaataaacattcaccatttttattccagccattactgtccttgtgcctcttactggaacctgtactttcatgctcagcaggtgtccagcattaaaagaaaaagtaaagattacctagaaagaactcctcaacagtagtgccacccaccatcctagaggtcgtcatagtgtttgtagctggcctttcttccccttgagaattctccgttggtttccgtgatttggttatcaacagtcctgcctgctcgcttgctgtcctgtgtagcttttgctgcttaggtgctgagtggttctatatttctttcccagtcctcttttgagtgcctggctgacattttcaatctctattgggctccaaaccaaaccagtttcgtggtattgtcctccaaaccttgccctcttatagcatgaacaatgtgttgagcatggggtattataagagttctcatttagcattccacagttgaggaatgtgtgttacttcaattacctttgagctgtagaaaaatctttagctgtggtaacagccacttctaggagaggagaaaatacggatcaactagcccaatttgcgatgttaggaatttgtcgattttcttagtaggatggctttcaaaggttagagcatcagagtcacctgaagcccgactttaactgtaatggtttaagatggggttgatggggaaacttgtagtacccctcaggtaattctgatactgcagcaaggtttgagaattcacaaagtctttttatttttcctcccgagatagtctcattctgtcgcccaggctggaatacagtggcatgatctcagctcactgcaacctccgcctcccaggttcaagcaattgtcctgtctcaacctcctgagtagctgggatgacagatgtgtgccaccacacctggctaatttttgtatttttagtagagatggggtttctccgtgttagccaggctagtctcgaactcctgacctcaggtgacccaccggccttggcctcacaaatcagtttttaattaaaaataagcaggaggctgagtgtggtggctcacacctgtaatcctagcactttgggagcccgaggaaggtggatcacttgagctcatgagtttgagaccagcctgggcaacatggagagaccttgtccctataaaaaaaaaaaaaaaaaaatatatatatatatatatatatatatatatatatatagtgtgtgtgtgtgtgtatatatatacacacacacacacaaaattagccaggtgtggtggcgtgtgcctgtagtcccagctactggggaggctgaggagggaggatggcttgagtctgggaagtggagattgcagtgagctgagactatgcccctgcattccatccagcctgggtgacacagccagaccctgtctcaaaaataataataatcagtaaacccagtgtggggttattcctttagattactattattttgttcttgaacaattgatttttatttttttagactttttagcctttatataatcattctgtgtactctgccttcataataaaactggaaaaattatgagcaagaaataagaggtactagttctgaggaatagttaagattatcatactgagtccaattgtagcagaattttttgttgcttctttgtatgatacttaaaatagttgaaaatttgattggattaaagagcatattggatcgctggagtatctgatgctagtaacattctgaacattctgcctgttaatgtgcccgtcaaaggaagtaaatattaataaaacttcttcattgagaatataaccggtttggcttttgtaatgccattatattcattatattaattttcatatgctgaaaaatgtcctcatgcggaaatgtggggtacatgacagggaaaagtttctggttttggattacttctgtcaaagctcagtactcgcagtcttgtatttaatcctctccctttgctactttccctaccaggatgttgcttcagagtgtgaagtcaaatgcatgccaacattccagttttttaagaagggacaaaaggtacgtacatctgacctttaaaactctaactgggcaataggaaacccagtataagtgaataaatcactggagtgatgttccctttaaagattgaggcatatcaccaagttctgcttttaagaatttttaaatatgccaaaattcattggcttaagtacataatgtgacagctaactgaaaatcaatctttcctagaactagtcctatttatatcataaagcacatagaatttcttagacttgggcagttcatttgttgttaagtattgtgtaaaagaaaatttgtacttgagccttttgacttttctcttgatattttttctttgtttataacttaaatgaactgtatgttattcagggaagtttactttaaataagattatacttctttttccctccacccctattcttccttcattctatgctgaatacatatttatacatatgtatatatatacatatgtatatgtatatatataaatacatatttatacatattttatgtataaaacagtgctacagtgctacgtctaatgtcaattcaatattctcttaacaggtgggtgaattttctggagccaataaggaaaagcttgaagccaccattaatgaattagtctaatcatgttttctgaaaatataaccagccattggctatttaaaacttgtaatttttttaatttacaaaaatataaaatatgaagacataaacccagttgccatctgcgtgacaataaaacattaatgctaacactttttaaaaccgtctcatgtctgaatagctttcaaaataaatgtgaaatggtcatttaatgtattttcctatattctcaatcactttttagtaaccttgtaggccactgattattttaagattttaaaaattattattgctaccttaatgtattgctacaaaaatctcttgttgggggcaatgcaggtaataaagtagtatgttgttatttgt

SEQ ID NO: 7 - protein, 284 aa, Homo sapiens
MEAIKKKMQMLKLDKENALDRAEQAEAEQKQAEERSKQLEDELAAMQKKLKGTEDELDKYSEALKDAQEKLELAEKKAADAEAEVASLNRRIQLVEEELDRAQERLATALQKLEEAEKAADESERGMKVIENRALKDEEKMELQEIQLKEAKHIAEEADRKYEEVARKLVIIEGDLERTEERAELAESKCSELEEELKNVTNNLKSLEAQAEKYSQKEDKYEEEIKILTDKLKEAETRAEFAERSVAKLEKTIDDLEDELYAQKLKYKAISEELDHALNDMTSI

SEQ ID NO: 8 - DNA, 36830 bp, Homo sapiens
agataaagactcaagtctggggacctcctggtcactcaggcagcagccccttctttcttgccccagtctccagttctccagtgttcacaggtgagcctaccaacagccactgctcatgatggaggccatcaagaaaaagatgcagatgctgaagttagacaaggagaatgctctggatcgggcagagcaagctgaagctgagcagaagcaggcagaagaaagaagtaaacaggtaggcactggcattgatctctctcatctgctagtgaacaagactgtgaaatggaagggagttttcatgggaaagacagtgactactggtgtcacctcctttttgggggtgggatggactccccagcctcactggaggagcagaggatatctaacatatctgtctcagactgcctttgtgaccttgggaaagtcagtgagccacagtatgtctgctgaatttaagtagaaatggaccatgttttggaagaacccttgacttctagaggaaaaggtaagagttctagagggaaagagcatctcttttcactcaaatagcatcacgggcatctacactttagatatttatcatccaggggtcttggatgaaaacccaggatggcttggtcatcttaggatcagagattacagggtcacaggccaagctacggcataactcctgaggcagtgggctggaagtggctggggagaaagtgtgtctcactagggatggtaacatggaggttacactcagggctccaagagcccagggtccaaccctgcattgttgtgtttgtgtgtttttgtgtgtgactctctgggctcctatagctggaggatgagctggcagccatgcagaagaagctgaaagggacagaggatgagctggacaagtattctgaagctttgaaggatgcccaggagaagctggaactggcagagaagaaggctgctgatgtgagtattagagagatggaggatgggtttaatctacacatatagatctttatgttaatgtatgacgaaattccaaacacacttgtgggttcatggaacacagacatatatactcacatacatttttgcatataaccacatgctggtatattctcataatggcatgtattttctttcttaaagacatctccaacatgttaaaagtttttttttaatacatgtaaaggtctcatttatctcctatcaccaacccaggaatttaattatctttcccaggacctggacaaagagaggggactctaagttcagaatgctgtcacttatcctacctcctcccccatgccacattcttagcttttatgtccaagtcttggcatgtggatatgtgtattaatagggaacctctcatctctcaactgtccaacaaaaccagctcggatttcacaatgggaaatggaggtcacactgaccctggccatatatgggtttatttatcttggccaatgcaggcatttgcctttatgccatggagatgccatatgaagcattgctcctagggcagcagagagatccctacagtgacagagtataatagaaactaagaaagaatcttagggggtaaaggtatcaccaccagacttcactctatgaccgtgagtaattactcagcttgtctagggctgaattttcccattctcaaataaaataaattctcttttccatctcatattatcccatcatcatcacctactttccttcatctttaaccttgaagtttgctgcaaagataaatgagagagtagattgaaaagttttttagaagaaaagtaccataggaatcttaccaacaacaacagaaatgtcccagtattggtggaaacaagtttctcagtgaactgagttaataggaaaaaaggagcatgtaatatttaaaatcatatttgggctgggtgcggtggctcacgcctgtaatcccagcactttggaaggccgaggcgggtggatcacctgaggtcaagagtttgagaccagcctggccaacatggtgaaaccctgtctctagtaaaaatacaaaaattagccaggtgtggtggcgggtgcctgtaatcccagctactcaggaggctgaggcaggagaatcgcttgaacccaggaggcggaggctgcatggagccgagatcgtaccactgcactctagcctgggcaacagagagagactctgtctcaaaaaataagtcaataaaataaaatcacatttggttttggaaaatagctagaaggacagacagagtgaaccattcataaatgctttcggatcctcatatttggtctctctgccatccggtttctttccttttcttttccttttttttttttttttttttttgagacagggtttcactcttgttgcccaggctgtaataaaatggtactatctcggctcactgccatctccacctcccaggttgaagcgattctcctatctcagcctcctgagtaactgggattacaggcacgtgccaccatgcctggctggctaattttcatatttttactagagacggggtttcatcatattggtcaggcttgtcttgaactcctgacctcaggtgatccgcctgcctcggcctcccaaagtgctgggattacaggcgtgagccaccccgcccagccaggtttctttttattgttgttgttgagatggagtctcgctctgttgcccaggctggagtgcagtggcacaatctaggctcactgcaagctgtgcctcctggattcaagccattctcctgcctcagccttccgagtagctgggattgcaggtgtgtgccaccatacccaactaatttttgtattttttttttagtaaagatggggtttcgccatgttggccatgctggtctcgaactcctaacctcaagtgatccgcctgcctcagtgtcccaaagtactgggattacaggcgtgagccactgcgcccagcctaagccactgcgcctgggcttttttttttttttttttttttttttttttttttttttttttttttttttgagacagagtcttgttctatcaccaaggctggaatgcagtggcaagatctcgactcactgcaacctccacctcccgggttcaagtgattctcctgcctcagtctcctgagtagctgggattacaggcagctgccaccatgcccgggtaattttttatatttttagtagagacagtgtttcaccatgttggccaggctggtctccaactcctgacctcaagtgatccacccaccttggcctctcaaaatgctgggattacagatgtgagccaccatgcctggcctctgccattaggtttctgatctgttttgttttccaccactttcagtggccaaggaaagtggtaagaattgaatatattatgccagaggttagaaacacaaactaaatatacatcatttaaactcattcttccaaaagttaaggccagcacaggcttttttttttttttttttttgagacggagtcttgctgtgtcacccaggctggagtgcagtggtacgatcttggctcactgcaagctccgcctcttgggttcacgccattctcctgcctcagcctcctgagtagctgggactacaggtgcccgccaccgcgcccggctaattttttgtatttttagtagagatggggtttcaccgtgttagccaggatggtctcgatctcctcaccttgtgatccgcccgccacggcctcccaaagtgctgggattacaggtgtgagccaccgcgcccggccaggccgccacaaactattaattctcccttcttgtgcccagtgtggtggctcagacctgtaattccagcactttgctttgagaggccagggcaggagaatcgcctgaggccaggagttcaagaccagcctggggaacatagtgaggccctatctttacaaaaaatttaaaaattagccaggtgtggtggcatgtgccggtagtcccgcgtactccagaggctgagatgggaggatcgcttgaagccaggagttcatgcttgcagtgaggtatgataatgctactgcactccagcctaggtgacagagcaagaccctgtctctatttaaaatgataaaaattctcccttctcagaaaactcaaaaaaacaaaacaaaacaaacaaacaaacaaccctaccaaatctacaggttacccctgggctttgggcaagtggatggctggctggagttgtatagcctaggttcctggtatatttggatggatctctctgtttctaacatatgggcatgaagatagaagttttcacaaactagcacaatgtcaggttaataacagttttgtgagaaattgggtaacatcaatctgggtctgctcttaggctgaaacggctagtatgtgggcttgaggaggtgtttccaggagcaagaaaaagaacaagtctaacctgagatcatgagaaggagaataacatagaggctgtacgcaatacatcaaaatcattaggaatataaaaggatttcctggggatgaattctgtctatcttaatctgttttacacatgtacagacatacacatacacacacatagcacccctcacctagatgtggtctactcagtatattcatttgaaagaagtttcttcatatttacataggaatccaccaaaacccatttaaatatgtaaagtgagggttaaaaatactcattcaacccctctcccctttgttagctttcccaagtcagaaagtaccctcagagatatgcataagaacaagaaacaggagagggaaggttgtggccaggagcaagcaagaagaaaggtttctttcctcaaagctagagtcagcaaactaccacctgtgggttaaatcttgcaacagcctggttttgtaaataaagttttatcggaacacagtcatgctcattcttgtacatactgtctactggctttcttatctctctggccacaagaccctaaaagttggatgacccctgcctcaaagggtcaaagaaggacagtgtttataggaattgggagaggtgctgaataaggaccaacaaggttaaaagcagaaagcaaaggtaagagtcatcagcagcataatatatgcatctttggagggagtatactctgtccaatacaaaggaagttaacagctcagcttctgtccttgaggttaataaataagtaaacagagcatattatcttgaagtagatataagcagtagagaaggaggagggagtcaataactacgtgtttacacaagcagttctggttatctgagaagcctttctgaaaagtgcaggatttactaggactgcttgaataaagaaagggcaagggtatggcaaggcgagtccaaacttttttttcctccctaaatttgtctaactttgttttctaagttgtggtaaaatacgcatacataaaacttaccattttaaccatttttaaatgtctaattcagtagtagcatatttacattgttgtgcaaccacatctaagatcattttaatccaagatgaaatcagatgctgtttagggagaaacaaagggaaagaggagtgcattgttaggacatagagtcataacaccattttttagggacaggtggcaggaaggatggggagagaagggacaacgatggacatatacacctgttgatatattgtattgctggttactaggaacacaatagcactgggccccttgctgtctatccattgtgcttctaaaggctatcatgagtcagtccatctgtttgactgcattaagaggagattatctgctcaaatctaccagtctcagaaatgcagtggtgctctcaataccctcttggtttggctaggatgaggtcttctgctgctttaccagggcacagggaggtacaatagtgccccctgctgagcagcacccagatgacaaagtgtcacttctgccacatcttgatcagacttctacaaagcaagttagaaaaattcttaggccatcagggtagagtctattaagagtcagaaccctgatcttccttcctaatttctgggggaaaaatcagtgtgcaggttactctattgagaaaattattatctatccaagttgtttcttctgaatagtctcattgctcggtatttccaaaaggtgataggaaaacagaggctctgcagggagcattcttagaactagacattgagggcagcaactgtcttgcctactattcctttcagggattaggactcttcattgtaagtcctgttccacatcctaattttctaggagaaaagagagctgtcccagaagaaaaatttatcacgattacctactgggaaggtgggaggaatacaaagaaataagagggaagataacgttctcaagtctctttactattgtgcttccagaaatcctgcatggcagtgcctgtagggaaagtttctttttttgttgtttttctttttttttttttcagactgtgtctcactctgtcacccaggctggagtgcagtggcgcgatctcggctcactgcaacttctgccttctgggttcaagcaattctcctgcctcagcctcccgagtggctgggattacaggcgcctgctactgcacctggctagttttttgtttgtttgtttgtttgcttgtttttgtggtttttagtagagacagggtttcaccatcttggccaggctggtcttgaactcctgacctcgtgatccacctgcctcagcctcccaaagtgctgggattacaggtgtgagccactgcgcctggtctgttttgccatttttttttttttttttttgatacagagtctcactctgtcacccgggctggagtagagtggcaccatctcagctcactgcaacctctgcctcctggatttaagcaattctcttgcctcagcctccctgaatagctggaactacaggtgtgcaccaccacacccggctaatttttgcatttttagtagagacggggttttgccatgttggccaggctggtctcaaactcctgacctcaggtgatctgcccgccgtggcctcccaaagtgctgggattacaggcatgagccaccacatctggccaaagggaagtttctaactcataaacagttccttgggggtacttggaagtacagtggcattgaaattggtaaaagttgtaacagaatggaggctcaattctatagtacagtttacaggcactcatgtctggctgttgcatgaaggatctagtaacagtcgtctgttttttatttttttgagacaggctctcaatctgtcacccagactggaggcagtgatacaatcttggctcactgcaacctctgccttcccggctcaagaaatcttcccacctcagcctcccaagtagctgggactacagacgtgctgccaccaagcctggctaattttttaattgtttgtagagacgaggtctcactatactgcccaggctggtctcaaactctgggctgaagtgatcttctcgcgttggcctcccaaatgttgggatcacaggcatgagtcaccccgcccggctgacagtggtctgtaatagaggatttcttcctgataaaatgggggcatatctttcttacctacaaaaggaattgtaagtgattattaaattatttatttatttttagagatgaggtcttgctatattgcccaggctggttttgaactcctgggctcaagccatcctcccacctcagcctcccaaaatcctagacttacagacatgagctaccacgcccagctgatgattaaattgccaagtcctgaatcattccaaggtttggaggggctggaaggttggttaatttggtgcatgatgactctcaaaagtaacagggacctgtaatcccagcactttgggaggccgaggccggcagatcacgaggtcaggagatcgagaccatcctggctaacacggtgaaaccccgtctctactaaaactacaaaaaaaaaattagccgggcgtggtggagggcgctactcgggaggctgaggcaggagaatcgcttgaacccgggaggcggagcttacagtgagccgagttcgcaccactgcactccaacctgggcgacagaggagactccgtcccaaaaaataaaaaacaaaacaaaaataaataaaataacagggagcctgacggggtggcgtgcacctgtagtcccagctacaccggaggctgaggtaggaggctcgcttgagcccaggagttcgaagctgtagtgagttgtgatctcgccactgccgcactccagcaacagagcgagatcttgtctcttaaaaaaaaaaaaaaaaaaaaaaaaaaggagaaaagtctgttctaaatagaacacttaagttcataccatgccaaatagaatgagaatttgtgccatcttgcccatcccttccactccgacagttccattcattgacttccaggagacgggagagagaacctacctacctgagccgactttgaaaggagactgagtggggctcttttgaacctgaggcccagaaagtcttttagacttttcttataacttggttttggagtttgtcgtggggtgagtggaagcagagacttgtaggatgtttatattgtggagcaaatacagtgaggggtgacgggtatcggcattgcgggagtgaggggtcaattgcgggagtgtaaggtcagccgactacctgcaaaggggccaaccattgtgggcagccagcctggcgggagaggctagggcacgaagcgccgcccagttaccatggtaacactccctcgtcccaaccccgtcccagtttactggcccaaggtaaggccgggagccgtactccagggcgactgggaggatccactccctgccgcggctctccctccagtggactgccttcccgcctgcctggcggtgggagggcgcggcagagggacgtcacatccgggcgggttggtgagttccggtatttcagggcgtagcaggcggaagtaagggtgagaggaggctgcaacgccgagcggaggaggcaggaaccggagcgcgagcagtagctgggtgggcaccatggctgggatcaccaccatcgaggcggtgaagcgcaagatccaggttctgcagcagcaggcagatgatgcagaggagcgagctgagcgcctccagcgagaagttgagggagaaaggcgggcccgggaacaggtacggagatggtgaggctcatgagggaggggcaggcgctggagtatccggcaagcggaataaggtcccttttgccgtggagcacggtcctctgaaggccggcgggctcggagatgaagttgggatctcggaggatgggtggggctcagcaccagtaggacaccaagcggacgagggggaaacagtgctcgggcgagcaggcttgacctttgaagaaattgagccttacggggtttggggtgtggctttcctcctctttttttctccacctcattcattcagtgcctccgatccgggcgcggccgtgggaggggctcagcggcttctccctcccccacttccccatggagggtgggagaactttgctctggcttgaggtttcctgcccaaggggtaagagtgggtgggtgcttctctgttcggggaggggcagtactgaaccttctgtgaaatggtgagggttttggggtgggaaccgtcggaaagaccccggcctgcctgctggcagcttttccgctccctcctcctgctcaggaaatgaggcagcagttggctgagaggaagcgcggtgccctccctccctggaggagctcgaggccacaggaccgcctctcatcctctggccaggggccctttcctcttacggggtgctcttgcagctgctacacccactctctgaatctacgtgaaacggcaggggaggggatagtacttctctcaacgttagtgcaaatgtaggagccaaccaaacttgcctggccctacccacaatgacctggaaggcaggaacttctgtcagattcaggcctttattgctgaagtgtgatcagtgcaagccgaactggttaagaacatttcttccgagcagagctgataagcgagtgtatggcagagaatcagatggtgtgccagtctcaaggtttctgattgacttgggcaatttggtggagacaataatgaattccaggtgactctagtggaatatttgttcaactgaaagtggaaacataaaatgttggtggagtgaacagtgattctagtgatttctttcttcctttcttttttttgagacagagtctcactctgtcgctcaggctggagtgcaatggcatgatctcggctcattgcaacctccgcctcccgggttcaagtgattctcctgcctcagcctcctgagtagctgggattacaggtgcccgccaccaagtctggctaattttttgtatttttggtagagacggggtttcgccatgttggccaggctggtctcgaactcctgacctcaggtgatccacccacctctgcctcccaaagtgctgggattacaggtgtgatccaccgtgcccagcctattctagtgatttctttaaacgtctttctgtttaaaagtggctgattagttttagtgtaagccataagattttccaaggtctagggtgcagtctaatagatggagctgatacgagttgaggctttgttttggaggatcttgagagagtaaagtaactgttgctaaaattcaagatccagtgaaagtactcatgtggtttattctttgttctttgttctttcccttggtaagtttttgtttttgtttttctttttggctgctcctctgggaaataactgagaatgtttttgggagggggaggggtggatttcagaaaaggatggtaattgggggacagctgccctccagtttaaatacttaaagattgttggaatactcacttatttttataagtggtcatggagatgctgggatgggagcttgttgtcttcctcagacacctttttctaggtagatttttttcctctgttcagtttgccaacaacttcttattatctgtgcaagtgagatggactggctgggtgactctttatggccagtggcactttctcatctcctttgttctggagaccagatagctttaaaggggaagacagaggaactggccatgtgttgagctgcagaatcttaggctcagtatttaaatatggacattctaggagtagttctaaggtgggtgattcctgcctcccatcccccctctttttttttttagatggagacttgctctgtagcccagcctggaatgcagttgcacgatctctgctcactgcaacctccacctcccaggctcaagtgattctcctgccttagcctcctgagtggctgggattacaggcatccaccaccacactcggcgatttttttttattattttatttttttatttttagtagagatggggtttcaccatgttggccaggctggtttcaaactcctgacctcaagtgatctgcctgcctcagcctcccaaagtgctgagattacaggcatgagccactgcgcctgtcctggggtgatgtttcttaagctactttcactcagagcctgccaaatttgcttattgagctgaggtctagtgaggaagtcctcagaatggggaagtagagttatagttaaatattgatgaaatcatttcttctactccccagcctgaagaagcttaatcttttgactttctgctgctctgctgaactttgaacccctactgaggaggagacacctgtgggaagggaggggggttgcttgggctacctgttatcctcttaaggcattctggaatgtgggttttgcagactcctccctcctctggagcgggaaactgcactctctgatggtatctcatgctgttagtgggattgtcacttaattattttcctggttactttgtgtgaacatccaagccacccttgttgggaagctagatgagtctaaaactgagacactggtgttctaggatccatctttcttttctgctctcggcctggaaattggtcttttataatgtagcaccttgtcaaatttactttcagttctgtatttcctttttacttctgtctaaatggaaacagatggcaacataatgtttaaatgcagtctttttccttatctctttttttttgtggtagtggtgataaaaatacatgtaaaatttaccattttaaccatttataaatgtacagttcaccttatctgtttttaataactctctcctaattgtccctgatatttttctctttttaatttttttctagtctaaaaaacttaagaggctgggtgcggtggctcacacctgtaatcccagccctttgggaggcctaggcgggtggatctgaggtccacctgtggatctgatgaaaccccatctctactaaaaatacaaaaaattagctgggcatggtggcgcatgcctgtaatcccagctacttgggaggctgaggcacaagaattgcttcaaccctggaggcggaggtggcagtgaaccaggattggatcatgccactgtactccagcctgggcgagtatcggcactgggggagtgaggggtcaactgcgggagtgtaaggtcagcacactacctctgcctcaaaaagaaaaaaaaaagtaataacaaaaaccttaagagtactaccgagggagtgggctttgattctggccttaatcctaaattcctagtctctggagaggttagaatgcttgactgttgtgtggttctcttctattcgtgggcctggtggactaagcaacctttctttggttgctttgctttcctttctctttccccagtactagaacacacactcacaggggagaggcagatagccctttacccctgcttaggctccgcctttgtttactgtggggcagatagctctactcccttccaacttgcctcccctttggtgaagtttaaattccccaaggacacggtatctccctgggtaatgcatttgtgagcacagtgaactcaaatagtcctttactcacagggctcaagcctaactttctgatactctttcatttgaattttatctgtggcttttatctaataagagaaaccccagtgtggcagaaagccagatttaggctaatccccttctgtccttggccctgttctgcctgagagtaccatcacactgtcagtttcttgcctggaggggttgacatatttgggaaatctctttggagtggaggatagccgaaggtgttgtcagccatctggtaatgtgactgagttgctggggcattctactctttccccaatccctcacccaggatctttgcttcctgggcagtggagaatcctattattacttgaagggaagaaccacagagtactgtatatcctccacactgggcagagataggtcagctgaaacaccaaaaccgcatctggatgggcatgctaaaacctaagatgattaggaaagtgcagagcttaacgtttcccagggccacaggtagcctggtctgcaaaaagtaggcctgttcaatctcttggtacccattggtattatttgacctgaaagggacaggcactgtgcatagactcatgagggaaaagtcagcttgtcttccttgttcctttaaacaaagtatcagatcttgattctgcaggcttttgtttcattgtaaagagaactgatcctctgtgctaggagaaaagagaaaggcaaggtcccattccctaccactccaccctggagggggtctctgaagctttttctaggactcccatctcaggttctgcttttgatacaggcttgttcatgtgtgcatgctcttaggagggcaccaagatgtgttttccccctctcccttttcagccttgctaagaattctattgtaaatgctgatttgtgggagaaactgcctgagccagcttgctgtcatggcaactgtacacccccgccccatagcctgttatcttctactccagtttgggtaactactggtgagctgcatgatgtcttacatttcatgagcttcctctaaatctagttgtatttttcgtctgaggggggtaagtcattttactagcagtgtctcctaggtcttgcctgatagcctgacttgctcttgatccatggccttgttgggtttgtgaggagaaatgtgtgttgttacagagaacatctatttttctggaagaggcagaagcagagaatacttgtctttgtggtgttttgagtagtgtagttagaggcacaaatggtttcttaatgatttcagcttatgttgacttctcgattttctttgcctggtggctagattgtagccccagccctctgcttcccttcctagtctcttcagcttctcatgtttgtatgtggaagtttttgtgccagtcctgatggttaaaaaggagtcttttggtctatattcttcctcatcggagctagtggcccaggctgtttctatagctgcaccaactcctgcctgccaggaagaaatcagggtaatagaattcaggcacaatctgggctatagaagagagatcgtgtttaccaggcacttggataagcatctgaactgggtggtgtgcggggcatattaaattcttgtatactgttgcagtcagggtacctcccaccagtaaaggtagcggggaggcagtcatggaaagaggttttcctaccattagaatttatccatccacctgcaggctgcctccttctttccttgtttcttagagaaaacccatccaggatgggtttacttggactctaacctatatacttcattgtgagatggaggccagggggaagattgtcgctgtattagagggtgaagctaaggggatggctggaggaatagaaaggagaaaattcagaaatttggaggaatggtagtgggtgtggtggaaatgttctttcttttttgtaaagctatcaaagcagataattttagccagaagcagctccttgtagccaacttaaaaaaggagtacgggggcacaagttgaagggctggtgagtaggcatcccaacaatattccattcttttgctttccagtctggaaggctattctggaagacattcccttggctagttattcttctgatgtgttagggtaaagtgatcataggaataacggtttgagttaagcagcaattaataggtaatccaggattccctctttttttttttttttttttgaaatggagtttcaccgtgttgcccaggctggagtgtagtggcgcaatctcagctcagtacaacctctgcctcccgggttcaagggattcttctgcctcagcctcccgagtagctgggactagaggtacgcgccaccacacctggctaatttttgtattttttattagagtcagggtttcaccatattggccaggctggtctcaaattcctgacctcatgatccacccgccccagcctcccagaatactgggattacaggcatgagccaccacacccagtcaggataccctctttaagaaaccttgcttttttttttttttttttcctgctttatgagacacttgaaaggttactgaggatgagacatgcctccaagccagagctcaggcagaaggaacccttcattgactttcttttctgactccagtgtgttgaggaatttgaactgagtccaggaagtgggtggtactaactcactgggctgggcaaggaactgagttttaaaacactcttcctgtggaacatatgggagttcgttgatgggaaagggaggaaatatatatatataaagttatgtctgctagatcttttaacagctgagctaagacaagttctaccctgcgagactctggctttttcatggtagtcaaccctgatcttaatgtcatagttatttcttcttttctccatgcctctgcttcccttgtccattctttccttcaaatgcaggctgaggctgaggtggcctccttgaaccgtaggatccagctggttgaagaagagctggaccgtgctcaggagcgcctggccactgccctgcaaaagctggaagaagctgaaaaagctgctgatgagagtgagaggtgtgtagggaagcctgatggagtgtggattttaaaagttcataatagttctgatcaagatttccttatagacctgttttctaaatagcaattctggctgcgtgtgatggctcacgcctgtaatcccagcactttgggacgctgaggcaggagggtggctcaagcccaggaagttggggctacagtgagctctgattgcgccactgcactcctgggtgacagagaaagaccctgtgtcaatcagtcaataaaaataaaatactaactccatcacacaggatctcctttctattctgcatattctgtccccttgaagacagtaagggtaacttagatgttgtgacccttttgtaattggtatttccttacaaattggcctaatgatccttaaaatgggatcaccagtatgacttctactttctggatcataagcttctatttgtgagattaatttctctaaaacagaggttctctaagtaaggtctcagaccagcagcatcagcaatacttgggaacttggtaaaaattcacattctcagctcctatgctgatctgctgaatcagaaactttggatgtggggcctggcaatctgtttttagtaagccttctcaacgctggttgcattattagaatcataggtaggggtgaggggggaaatcactggggaatttaaaactggatgcttagatctcatcccagttccattagaatcagttgcaagaggggtctgggcattgatgtttttaaagttctccagctaattttttttttttttttttttttgagacagagtctcgctctatagcccaggctggagtgcagtggcgcaatctgggctcactgcaagctccgcctcccgggttcacgccattctcctgcctcagcctcccaagtagctgggactacaggtgtccgccaccacgcccggctaattttttgtatttttagtagagacagggtttcaccgtattagccaggatggtcttgatctcctgcctttgtgatcctcccgcctcggcctcccagtgtgctgggattacaggcgtgagccactgcgcccggccttccccagctaattttaatgtgttcctagggttgagaaccatggcccttgaaacaaattataactcatgtttttgaaaatagccagttgtctccttgccacatgtgagttaaactagaacagaggtgcttgacaactaagggttttcagaataaatagtgcctttctaagggtacgttactactgtgcagctgtgaaaatgaaagaggcagctatgtgtatactattagagtagcatgcaaattatagaaagtgagggccgggcgcagtggctcacacctgtaatcccagcactttgggagcctgaggtggacgatcacttgaggccaggagttcaagaccaccccagccaacacagcaaaatccagtctctggtaaaagtacaaaaattagccaggcgtggtggggcgcgcctgtaatcccagctactcaggaggctaaggcacgagaagcactggaacccaagaggcggaggttgcagtgagcctccctcttttttttggtctcaaaaaaaaaaaaagaaagaaagtgaggatagctgcatgctgaataccggggggaaaaaaaagagtgtggaaaacaagttgcagaaccgtctagtatattattactttggggaagctgggatgggatttttgtgtgtatatatatatatatatatatatatatatatatatatatacacatatatttaaataatatttatttatttttttgagacagagtctcactctatcccctaggctggagtgcagtggcacaatctcggctcactgcaacctccacctcccgggttcaagccattctcatgcttcagcttccctcaagtagctgggattacaggcgcctgccaccacgcctgactaatttttgtatttctagtacagacagggttttgccatgttgaccaggctggtctcaaactcctgacctcaggtgatctgccggcttcggcctcccaaagtgctgggattacaggcgtgagccaccacgcccagctgtatatatccatatttttatttaatattttagtatttaatttcttaataagtttttttgggggtggtggaggagacggtgtctcactctgtcgcccaggctggagtgcagcagtgcgatcttggcttactgcaacctctgcctcccgggttcaagtgattatcttgcctcagtagatgggattacaggtgcttgccaccacacctggctaatttttgtatttttagtagggacagggtttcaccatgtttgccagctggtctcgaactcctgacctcaagtgatccacctgacttgacctctcaaagtactgggattacaggcatgagccactgcgcttggccaggatgtattacctttataacaaaaagttttttttttttttttgagacggagtttcgctcttattgcccaggctggagtgcaatggctcgatctcggctcaccgcaacctccacctactgagttcaagtgattctcctgcctgagcctcccaagtagctggaattacaggcatgtgccactacgcccagctaattttgtatttttagtagagacggggtttctccatgttggtcaggctggtcttgaactcccgacctcaggtgatctgcccgcctcagcctcccaaagtgctggggttataggcgtgagccactgtgcccagccataacaaaaaattttaaatgaaataattaaactaaggtgtgggagtagtaacatggaatttccatttgaggggcagacagagaggagtgggcttttaaaagtacagttctgagacccttggagtggagctgacgacacactcctcagggtgtcagtattgcttttgtcctatttctggcagaggtatgaaggttattgaaaaccgggccttaaaagatgaagaaaagatggaactccaggaaatccaactcaaagaagctaagcacattgcagaagaggcagataggaagtatgaagaggtaagtgaccttctgttagtccctctcgtatcggccttttacaaagcataatgcggtaaatggctcctttgcttcactaagctatactgggatcttttcctgtaggtggctcgtaagttggtgatcattgaaggagacttggaacgcacagaggaacgagctgagctggcagagtcgtgagtatctagctccccaaatcttgtcattaccaatttatcccttcccctttggcttgggcactaatatgaataaattagcattccttcaaccttgttaaacagggcctagggaatgggagtcatctatagtgcttttttaggcccaagaatggttttaggcttccttattctcctgactagtgacaagtgacaaaagaggctttcttattttttgttggtagggtttacaaattgagagcaaataaggactaatacatggtgaacagttaattggaagatcctactgggtccttccacctggggtggaagacatcaaagtggctatcctgatttaagatctagtgtctggctgggtgtggtggctcaaacctgtagtcccagctacttagtatgccaaggcaggaaggttgcttgaacccaggagttggaggccagcctgggcgtcatagggagaccctgtccctaaaacaaataaatttaaaaaaaaaatctggtgtctgttttggccactttgccaatctgctagggaaccttagggcaagggtagatattaactgctctcccgtcggtaccttctgggccttgattaacaaagtcagtgctgaatgcatatgtatttgtgcttccctcttacattcccgtctgccagacataacatatcatgaccatggtggtgttctcttcccattttttgaatttttttttttcgttttgctgcttgttttcttgctgtgttcctttttgacctttctccctccccacaacctggcttgtgccatccccgtgactatcactaacagccgttgccgagagatggatgagcagattagactgatggaccagaacctgaagtgtctgagtgctgctgaagaaaaggtactaaccattaagcccacagcaaggttatatatgtggtgtggttttgttttgttttgtttttgttttgcagctgcctcccctccacttgattttgttgagtgctgtctcatctcttcatggaatgctccattcttttccacttacttggcatctttcatctcttctttcctagttgcttcactgcctctccacagggtctccttcctttgggtgatttgggggactgtttgggttgggcatttgttcccacctccaccccaggtagcagttatgacttgttcctggtagtggatttctctcaagtgctttattagtattcaaacatttttgagcccaggaaggtctagctcctgacacgttctatggtagagggaggagggttgatgcttgctcaggttacttgggaacatctcttccccagtatgccttccaactctctctacatataggttcaattcacgtctatgtgtctctctccctttttttattctcttccttttcccttttcctctcctgtggtatttaaaatattcacagtaagtgttctgagctggaggaggagctgaagaatgtcaccaacaacctcaagtctcttgaggctcaggcggagaaggtaggggctggtttgtgaaagtgacaggctttggggcctgggcccagccctgcccttgatgaaagactggagacacgcattccatatataatccagtgaggcgtgtcctcttcctacctccttaccatgtattcaggttctgggtttaatgctattttctgtgcactttgattgctttaattaaaataacttcttggtctcaggttactggagtcatgtgctccttaggctgatactgctgtgctgaaaagcaataatcaagggccctaacaggctttctagcagacagatgggggcaacagacagaatcagctctgagacctaatgagcaatcaagacagatgacttcactttgccatcatgtgggtgtgtttcagtcatgtgaccttggggtgctactactcttgctcatcaagcaggggaaaggaaaaacacctccttgggttgatctgaataatatcaaagcttagatccatcttaaaaagtcatggagaattttaacctgaggatctcatggctctaaagacatagatttctgaatttcaaggacctattgtccagagccaggccttgctcttactcttggaccattcagcacatttagtgcaagaggatattcaatgtaagttaaatgtagggaggggggtattgcctgtgtgactaatttctactctgaaatttttaatctacttatcttacagtactctcaaaaagaagataaatatgaggaagaaatcaagattcttactgataaactcaaggaggtgagctgaggggatttatagggcagaacccaaaacatttagaggtatataaggcattaatatggatttaaactggctggtttgggttctgaaaggtccagaagtagaaggtggaagaaatagagattgttcttctttgtcttcatctctggttctgactaatttcatatttcccccaggcagagacccgtgctgagtttgctgagagatcggtagccaagctggaaaagacaattgatgacctggaaggtatggaacctggagtagggattgaaatagagaaatatagaggaagactgcccattaaccattaatctctttttctgtacaccaaaggaagttggtagtgacttggcatgtattaatatcatgtgcactgtcctatgccacagctaggcaaagtgggtcaaactctagaattggttttttaaatttaatcttttcctttaagaagactttaccaaatagtctgcaatttgtacccttcccttttgaggagaatgataatggctgcatttacctccaatttattagtcttaaacctttagtctgtgggtttaaatggaagccatgatttcagaagggcaagatgtatgtcatttcatggctactttccacagttctttttgccactcaagttgtcgggtatttatccaataaatgaagttgtggaataaccatatctgaacattgaccaatgtcaggagctttctaaatactttctgtccctgcaagttggtaactgccatccaccctgacaagggaacgtactctttgctttgagaactctgatgtatgctcctttggtacactctcttggcagagttgcaggggcagcctgtggagcagaggggccactaataggttcacattttgtaggcagagaagtgtctgaatatggatatatactctctccctttgctatttttgttcaattaagtgttatcactgctaggaattgggtacctggatgggagtggtatcctcttgagtgcttttggtaataggaatttctagttatgactgtgcccaggttctcagcccttgctgcagtcgatttggagtatcaagaaaggaagttggtcataggagagagtagatctgaaaatgtccagtcatggttgccagagtagatattttcctaactgtggtattagacccacagtcacactttcctaatttctaatgattccctctcttcatctgcttctgaaactcttcactctgtcccctcattcctccctgtggttcctgtgcctgtccagatgagctctatgcccagaaactgaagtacaaggccattagcgaggagctggaccacgccctcaatgacatgacctctatgtaactatctgacagtagagtggggctgggatcttggcttgtgggtggatgggggtacaagctatgcatagtggtgtgctttggttttatcctttggaaactagaatctccacccttatctttgaaacattggtgctggttatttgtttggcaaaagctttggaggcatggccgggcgcggtggctcacgcctgtaatcccagcactttgggaggccgaggcaggtggatcacgtgaggtcaggagttggagaccagcctggccaacatggtgaaaccccgtctctactaaaaatacaaaaagtagccaggcatggtggcagtgcacctgtaatcctagctactcaggaggctgagccaggagaatcgcttgaatctgggaggcagaggttacagtgagccaagatgtgccactgcatttcagcctgggcaacagagtgagaccccatctcaaaaaaaaaaaaaaagaaaaacattaaaggctgggcgtagtggctcacacctgtgatcccagcactttgggaggctgaggtgggcggatgactcgaggccaggagttcaagaccagcctggccaacatggcaaaaccctgtctctactaaaaaatatttaaaaaattagttgggcttggtggcgcatgtctataatcccaactactcgggaggctaaggcaggagaatcacttgaaaccaggagatggaggttgcagtgagccgagataatgccactgcactccagtctgggttgaagagcaagactgtctaagaaaaaaaagctctggaggcaattaatcttggatcagaaggagaaccctgactgacttgtaatttttatattttgtattcatagtttcttcattatactgtgatttttctatttgcttctcaaatttagtcttctcagaagggatactgctagaggtagaatccatacaactaaggaaatagggcccacagagccagtaacttgggccctgacacattaagacaaaattcaggcctcctgggtgtgtttaattggttccctgatattaaagttcagggaactacccaagatgggaaataccaaattcacctaagaattgagctgagtcccagaagcaagccaagtgataaacagcaccaaaaagagttgttggggcttcatctgtttgctgtggatccctgatccttgatgctaatctgcctctttgtatctttcccactaaccctgaaaagaagccacatttctcaggctgaagtgtctggctctcttttattattcctgctgccacctcttcctttttttcctcttcctttttcccagtttgctatctagattgatgctagtccttctcacctagagtatccttactttttcatacagataattatcaccgtttctgctctgttctggatctgccccctttactcctcggggaacccaaggccccactctcgctctggattccatttgggtcagcctggctggtccccaaggcattaggatgggggagcaaaaagcaacttatgtattttcttccacccccaccccaaattaaaatgttaagctgctggaaacctcatgccaccctgcatttgtgtcattgacaaagctgttgctgtccctaagaaggagccttgggggtgtgatgtggggaagagctattgtaggctccccctcctctgacttatgtaatcaaagccacttttgtgtgtgtctattttttcttgacatttaaactcagctgatctgattctaccagagtgatggatttagtacaggttactcaggatagtaattttagttatactcctcaagctgaacaagattaaattccttatttccaggttcttaaatcatcctgcctgcagtgtttccattctctcttcaggtattcctcctttggtgtggtgtcattgagaagccattgaagtgactctcaatacacattctgtacccttttaccggtggttcaaatggtgcatcctcagaacacccagtgaacccaatacattattgctaagattgactaattatgtcaactccagtcacagaaaaatacacaatggatagaattctggacggtttttttactttttcttctttaaacctttcttacatatttgagacttgctaccatttgcctgctagtgtgtgactagtgggatataagataagtgataaattattattgggaaaactaaaatgaccaatcatgcatatttcaaataatgtgcatatgaggctaatgatttattacatacataaatttctgctagtaaaattttccttggttcatattgtggaattaaatatcaacattttagaaattcccattataggccgggcgtggtagctcacgcctataatcccagcactttgggaggctggggcaggtggatcacctgaggtcatgagttcgagaccagctcggccaacatggtgaaaccccgtctctactaaaaatacaaaaattagctgggcatggtgggtgggtgcctgtaatcccagctactcgggaggctgaggcaggagaatcgcttgcacctgggaggcggaggttgcagtgagcggagatcgcgccactgcattccagcctgggcaacagagagactccatctcaaaaaaaaaaaaggaattcgcattccaatttacacagcagagatttcttaatagtatagctgtgaattatactaatccaagcacgtaagtgttgttcacttagtactttagtttcccagcagggtatgtatttaaaatttgcttttcttttagctgggtgcaatggcacttgcctgtactgcctgtagcttcttgggaggctgaggcaggaggattgcttgagcccaggagttctgcactgtagtgtgctatgcaggtcagatgtcctcactaaatttggcatcagtatggtgacctcctgggagtggagaacaacaaggttgcctaagaaggggtgaactggcccaggtcggaaatggagcaggtcaaaattcctgtgctgatcagtagtgggattgcacctataaatagccactgtagtactccagcctgggcaacacagcaagattccatctgttaaaaacacttttgctcttcttttaaacagatataatcaggtagggaagttttccttaatctaaaactttaagtcatatcaaattcacgtttccttttgtccattccatttcttactgccgcatagcctttgagattaaggttcagattcatttattcagctgacacttcctggtatcttctaggtataggatattaagatggagacagaaataaagatggaggcaccattcctactctcaagttgcttagaatctactaggagagaaaacatacataaagctacagatactgtgtgatgaatgccacaaataatagtgttatgggaattgagtagaggggacgagattgatcctagtgggaatgaattggaaaaactcatggaagaaggcatgtaatgggtacttcataaacatgttaaaacttttttctttttttctttttttttttttttttgagacggagtttgactcttgtcacctaggctggagttcagtggcctgatctcagctcactgcaacctctgcctcctgggttcaagcagttctcccacctcagcctcccgagtagctgagatcacaggcgcctgccaccacacccagctaatttttgtattattagtagaggtgaggtttcaccatgttggccaggctggtctggaactcctgacctcaagtgatccacccacctcagcctcccaaagtgatgggattacaggcttgagccactgcgcccagccaacatgttaaacttgcatattcatttttattgggttccaaaagtggatttttgacaagcagagttgggttgtgggatagtgaatattgagcattccattatttatttatttttctctttttgagatggagtctcgctctgtcacccaggctggatggagtgcagtggtgagatcatggctcactgcaacctctgcttcttgagttcaagcgattctcctgtctcagcttcccgaggagctgtgattacagatacccaccaccatgcccagctgatttttgtatttttagtacggacggggtttcagcatgttggccaggctggtcgcgaactcctgacctcaagtgatccacccgcctctgcctcccaaagtgttgggattacaggcgtgagccactgcacctggcctgagcattccatttaaagagaaacaaaataataaaggcattgatagagatgagaaggccacaatagctctaaaggtagatttagacagagtagggaacaggttgtgtcttaaaatgaggctgaggagttataattttacttggagttaatggggatgccactggagattttgaataggatatgttgtgatctgaataacatataagaagaattaatttggtaactaatttgcagattagcaaagtgattcactaacatggtgtttctcacatttcagccatttatgttaccatttttgatggctttttccatattcattgctgcctacattgttttcttcagatctactcatttccagatttattttgttcttacttgacacaattctattttgaaatcaaagttttgatgtgttagtgttttttcctaatcacattaaagtaactatagcagtgaatacaccacactttggaaaaaattgggttaggttaatttagttttctaggagagaataatcagggtctgagctagagtagtggcaggagagatagcactaaataaagacaggcattgtgaaaggcctggccacttgatgtgaggaggaaagacaaggatgactggtttttagagtaggagaattagccgggtgcagtggctcatgcctgtaatccctgcactttaggaggctgaggtgagaggatttcttgaatccaggagttcgagaccagcctggcaataaagtgagaacctgtctctacaaaatataaaaacttagccaaacatggtggtgcgtgcctgcagtcctatctacttggagggccgaagccagaggatcctttgagcccaggagtctgaggctgcagcaagctgtgatcacactgctgcactccatcctgggtggcagaatgagaccccccccaaaaaaagagtaggaaagtggtggggttaaagatgtagggagatcatgttgtcctggtttggggagctgggggaagggaaagaagatacggctgacaatctggcaggtatctgggtgaaaacaaaattctagggcgtgaggcagagaggttgacataagagagttgggaatccatatggggtacaggtagtttatgccatggaggtaaattagaaccatgaaggacaattgcaaatacaaaaaaaagaggccaggcacggtggctcacgcctgtaatcccagcactttgggaggccgaggtgggcggatcacgaggtcaggagatcaagaccatcctggctgacacgttgaaaccccgtctctactaaaaatacaaaaaattagctgggcgtggttgcgggcacctgtagtcccaactacttgggaggctgaggcaggagaatggcgtgaacccgggaggcggagcttgtagtgagctgagattgtgccacggcattccagcctgggtgacagagcaagactctgtctcaaaaaaaaattaaacaatgaggaaaacgttatgtaagaggaggaaccaaatggaactgattggagggaaagaagacggttgagtagttctttaaaaagttccgggttggatgctttctagtggtttttcaacagagaggctaagaaggaagataactgaaaatgggacatgcattgatactttagtaatttgagggtacattttgagtgttaagaagtgaagaggaggagtcattttcaggggcaaaagagtgtattggtaggaagattatataggcaatataatttactttagagcagggctttccaacagaacttttggtaatgatgtctagtacattagccacatgtggcagtcaagcactcaaaacatggctagtgtggctgaggggctaaaatttaattgtatttcattgtagttcatttaaatttggctacctgtggctagtggctattatattggacaatgcatttctaaatattcagatcctgggcagaagttggctttgacatgaagaaagacttcttagaaaggaaggaagtagaccgggcatggtggctcatgcctgtaatcccaacactttgggaagccgaggcaggtggatcacaaggtcaagagatcaagaccatcctggccaacgtggtgaaaacctgtctctactaaaaatacaaaaattagctggacatggtggcacatgcctgtagtcccaactactcgagaggctgaggcaggagagaaaaaaaagaaggaaataagagggagattgggagaaaagtaaggtgttatagctctgatctgtggtatctcctgttttccttgcggtatgactagtgtgagccaaggatagagaccaacaaactggggtaccaaagtggacaatgaagtactatgtaattagtgctaatgctaaattcattcctttgtttaaggcttaatatccttgcagaagccatcctgtgttacttgagcctggactagtattggggtgaagtcaagcatcagagtaaaacatttgtccccttaatgttaccccctcttaatttctaagaaccacaggcccattttgcccttccttgttaccatcattgagataaggaataagatgtagtaaccccaagttatctgataggtacaactgacctattctgtttggcagctttatcttagacctattctgtttggcagctttatcttagccccaaccaaatctcttcctttcacatggtgttggttgaatactatccaactccatggggttggtttgtgtttttttttttttttttttttgagacggagtcttgctctgttgcccaggctggagtgcggtggtgtgatcttggctcactgcaagctccgcctcctgggtttaagtgatcctcctgcctcagcctcctgaatagctgggattacaggcacgtgcaaccaagcccggctaatttttgtgtttttagtagagactaggtttcaacatgttaggctgatctcaaactcctgacctcgtgatctgcccgcctcggccttccaaagtgctggggttacagacttgagccactgcacctggccatggggttgtttcttaattagatatagctgaaaagaacgctagaccaaataggttctctgccttgccttttcgtttgttttgttttagctattatcagggaaccaaaaactttaaggagctagtactggtcttaatttttaataactagagatagcagagttagaaactaagttcaaagtgagagaacagctgcatttgtctttctgacctcatgcaattcctaggaaactctgtgttctgtgatttagtcaggcaataaaatgctctcactccttctctgtctctttatttcttccctaaatggaaaagaattgaccaggctgctttgagggataaagatcctgataacgcggctgggtgcggtggctcatgcctgtaatcccagcactttgggaggcgggcagatcacctgaggtcaggagttcaagaccagcctggccaatatggtgaaaccccatctctactaaaaaaaaaaaatgcaaaaattacctgggcgtggtggtgggtgcctgtaatcccagctactcaggaggctgaggcgagagaatcgcttgaacctgggaggtggaggttgcagtgaaccaaaatcgtaccattgcactccaacctgggcaacaagagtgaaactcaaaaaaaaaaaaaaaaaaaaaggatcctgataacatccttctttcccaagtgagaatcacacaaactaacttggttataggtattatctaaattcaggctgttcacgagatctatatgtaatgtaacatcacaggaaatttgagagattagttcacaaatttcaacacctgttattttgacaaggacgccccagtgaataagacaggtccaatctcaggcctcatgaagcttatataccattagtatagttttgagtattccttattcaaaatgcttgggagcagaagtgttttggatttttgctttttttttttttttttttttttaatatttgcattatactttccagttgagcatcccaaattcgaaaatcagaaatttataccaggcatggtgatgcacacctatagttcctgctactcaggaggctgaagcaagaggtttgcttgagccctaggagttcagggctgttgtgcactatgattaggcctatgaatagtcaccacactgtagcctggacaacacagtaagaccttgtctctaatacacacacacacaaacacacacatctgtatgtgtatgtaaaatccaaaatcctccaatgagcattttctttgagcatcatgttggcattcaaaaagtttcagattttggagcattttggattttggactttcagatttgggttctcagcctgtatagtgtttgtcaatggtgaacgtttgtcatttagagtaggacagttctgttgatgtacagaacaatcctgtgtattgctgtcccctgcccaacagatacctgtatgggccttctagtcactgtgatatcaaaacctcccatacatactcaaaatctccccttggagagaagagtgcacaataccacccctattgagaaccattggtttaatggggagtgcaagttaaaggttcaccctgattctactgtttaatacagctattatttttagtcattccctcccaggctttttacacatatatgctttctgttttgatagtgtacataaagtggcaagtattactgggaccatactggtctctagcttcccagtcaatctggggagagcctcagcccacaaggcagggtacctgagcaatgctcacatatgcagatgattttaggacagaaaagtatctttttcatattgagatcctgagaccattttgaaacgggctgtaaaatatagggagaaacttatttgtgaatttcagaggttgatactaggagtataccaggaagagtttggttttcctgtccccaaagttgtattatccctgtgctagtcagatgtatttgaatcttccatatcccactatctctttaaagtgtgcttcaaatggaaaatccagataaacctattagatttgagcacttagctgcttgtatcaggaatgcttgctaaattgtcctttagcctgatttgtaaaaaaaaaaaaaaaaaaaaaggaaataacttacttagtagcttaaataaatatgggatttgttttttaatcacatacatctagccatgagcaactgtggcctgttgtagctgggctttccatgattctcttggtcttttctcttaagattgaaatatgcaactctggtcatcacatccatgttgaaggcaagagggaaagctaaagttttcccagaagcccccagcagactatctttctacatctcattggttaaaactggatcatatgatcacccctagctgcaagggaggctgaaaaacaacaggatagtcttgagtagcttataccagtaatttgccttccctgggactaggcacactatttgttgactcttaacaggtttggggttcttttaaagaagaagaagaaattgaatatttattggctaggccactgaaatgggtctatcacacaatatctgtaacacaaagcattagaaagtataggagtgaatgagtgtggggatatttctcccagtatactgtttataatgctgtcttgaatgctttcacagaaaaatgttcagctgttcacttccattcctttcaaaccctgattattgtgaacacatttcttcgaagccaactgtattccttcctttgccatttcccttgtttcagtttctcaacccagggaataaatattgtgaaggaagcattttttctccctcaaaatgttgtcctaccaacttgtcagtctgaatttcctctgttggtctttccaccaactgactgactcttattgccctctatctgaatcctccttgcatccaccctgccttacagatgcatgttatatataccagctagttgaaagactcataatttacttctggtactctactaactttccctttttctctttctcctcctctttttcctctccttctcttgtgttcccctcctctccgttgctgctgcagagcgtctctacagccaacttgagcgaaaccgcctgctttctaatgagctgaagctaacgctgcatgatctgtgtgactgatgggcagggctcaatgatgcccattaaactgagcttactgctcacaccactgacctggaccccaacaaaaagctgattgtctttttaaaagttattattttgccctgagcaaattgcattttaattggggcagttagaatgttgatttcctaacagcattgtgaagttgaccattgtgaagtttctgtccctttagaagagattatgggtgaagaagggaggggcctgagagattatagtgagaaaacttgcgagaattttgttttccacccttatttgctgctctttcacttgggcactgactgtaggatatgttcccttgcatggatgtttttaacaataaaaggactgacttgacaagttgttgtaactgcttcatcggcaggcccaggaatgggtccttctgactgggtggaaaaaagggaagtgaaagaaaagttgtgggatatgaatatgggtctgtgttgccatcaccttctctgagttgaagatttgagtattttcctcacctctttagagcagtcagagtggtttgcttgctagacagattagattctccttaatgttcagctgctgattttctttctgacttttgcgtcctttttctggttttatgttaatttcaagtaactgtcacaagctagttctgttcaatagctctgcagcaatctcaaggtttgcttacaactacttgtttcagtagtattcttggctttgttttctttagagattatttgacttaactgtgagcgcccttttatttatcccatcagttattactttggcctctactttttcgaaaaaacatgtagtgcatgaggatcttcctgtgctctttataatctgagattctgatgtttctattgtttgcaatgttcaaactccggtgagccatttcaagagggtattgttatgtgggcaaaacctagaaaagtggatggctgatggttaaggcttgctctttcattgactgaaagctgaaagtgttggttgggtgtgggagggagaggaaatggctgataagggccctaactccctcacccaggaagtgcagcaacacctacaacttcagtaggcaagccaaaggccctacaaaactgggtgatgtaatagctcacttctgtggctgagaaggcagctgctttatcagtctgcagcttctctgcaacaggagcaagtctcaaagagcgggtagaccttgaaatttacttctagttcttgtaacttctctcctttacccccattagataaactgaaatgcaccaaagaggagcacctctgtacacaaaggatgctggaccagaccctgcttgacctgaatgagatgtagaacgccccagtcccaccctgctgctgctcctccctctgacccagactccgcctgaggccagcctgcgggaagctgacctttaactgagggctgatctttaactggaaggctgctttctcctttcaccaccccctccttccctgtgtctttttcgccaaactgtctctgcctcttcccggagaatccagctgggctagaggctgagcacctttggaaacaacatttaagggaatgtgagcacaatgcataatgtctttaaaaagcatgttgtgatgtacacattttgtaattaccttttttgttgttttgtagcaaccatttgtaaaacattccaaataattccacagtcctgaagcagcaatcgaatccctttctcacttttggaaggtgacttttcaccttaatgcatattcccctctccatagaggagaggaaaaggtgtaggcctgccttaccgagagccaaacagagcccagggagactccgctgtgggaaacctcattgttctgtacaaagtactagctaaaccagaaaggtgattccaggaggagttagccaaacaacaacaaaaacaaaaaatgtgctgttcaagttttcagctttaagatatctttggataatgttatttctattttttatttttttcattagaagttaccaaattaagatggtaagacctctgagaccaaaattttgtcccatctctaccccctcacaactgcttacagaatggatcatgtcccccttatgttgaggtgaccacttaattgctttcctgcctccttgaaagaaagaaagaaagaagactgtgtttttgccactgatttagccatgtgaaactcatctcattacccttttctgggtttgaagctgctgtctctagaagtgccatctcaattgtgctttgtatcagtcagtgctggagaaatcttgaatagcttatgtacaaaactttttaaattttatattattttgaaactttgctttgggtttgtggcaccctggccaccccatctggctgtgacagcctctgcagtccgtgggctggcagtttgttgatcttttaagtttccttccctacccagtccccattttctggtaaggtttctaggaggtctgttaggtgtacatcctgcagcttattggcttaaaatgtactctccttttatgtggtctctttggggccgattgggagaaagagaaatcaatagtgcaactgttttgatactgaatattgacaagtgtctttttgaaataaagaaccagtccctccaaccctcagacctatttgacttttatttattaaaactaaatgtgctttctccacagaagctatgaggtttgggttaaaaatagcatctttgtgggtggtagcaacaggatttattctttattattattatttttgagatgaagtttcattcttgttgcctgggctggagcgtaatggctcgatctcggctcactgcaacctccgcctcctggttcaagagattctcctgcctcagcctcccgagtagctgggattacaggcacctgccaccatgcccgggtaattttttatattttaagtagagacagggcttcaccatgttggccaggctggtctcgaactcctgaccttcaggtgatccacctgcctcagcttcccaaaatgctgggattacgggcgtgagccaccgcacccagctggagcaacaggatttaatatagagcaaatgtttagttttatcatctgtaaaatggagataagtattgtcagagtaaacatgaagattagaaagaacacttaatgtgctgggccttttataggttaacactgacatctcaggctgaactatatacattttccttcacaaccatatcaatccttataaactatggatttatgctccttaaaacaatatataatgctgatcactactataaatgcgtggttttaaccaactgtactgaaacagctttgagtttatattctgtttggatatttggagaaaacaacaagtgctctcaagagtatttgcttagaggccggctgtgtgagtggataactttgaaagctgcttttgagacgccagtgtctggcatttcctgcattctggcctggaggccggacgtgaatctgacttctagtaaaaatacacggttcccttgacaaagtcgagctgtttatcccagagactgcacaattttccgttgataggcatggaccaatgctaactggaaatcattgcaaaaagtttttttgtcgggcggagggtgtggtgttaagataaacagtgtgcaacagaagaaattaaaactggaagaaattaaagggttttttttagacttt

SEQ ID NO: 9 - DNA, 25 bp, Artificial Sequence
  Oligo
gtgctttgtatcagtcagtgctgga

SEQ ID NO: 10 - DNA, 25 bp, Artificial Sequence
  Oligo
aactggcttgataagaatcagactg

SEQ ID NO: 11 - DNA, 25 bp, Artificial Sequence
  Oligo
gcctggacctctggcaggtcaagtc

SEQ ID NO: 12 - DNA, 25 bp, Artificial Sequence
  Oligo
tttctttcattccctctctgaaaag